SEQUENCE LISTING (68 sequences)

SEQ ID NO: 1 - DNA, 4272 bp, Rattus norvegicus
  source 1..4272: mol_type = genomic DNA
agatctgcccatctcaggctagttaaattagttcatcccagtttggcccaacttaccccatctagagtagcgaaactaatctgagcctagctaagtccagtttagtttaatgtagcccagcttggcacaggctaatacatactgctacagtttggtctagcctaccctaattaagctgatccaggcctgggtagacctagctcatctcagcccagttaaggttatccagtacatctctttccagttcagctcaggttaccataccttatctcaattcagctcagctagtgtaattcatcttagttcatcccctacccctctagactccctgttgatcttaactcagtttagacatggccaacaaagcctggcccaactcaggccaggttagtgtagctcagcataagcagtctagccttgctcagtctagctcacccttcctcatctaaattcaactcagctatgccggccctgcagcaggtcccctcagctcacccaagtccaaccagttcagtctggctcatttaagtcttgacaatccccaattcatcccagctcagcttagcataactcaggcagtccattcttagcccaacccagtttagcccagtttatcccagttcatcctggctgtactcagtgcaactcgattcatgttctcccaggccacctcagcccagttcatggtagctcatctgagcccaacttatcccagctcatcccaaaccacctcacctaagccctgctcagcctagctcatctgagcctagttcaacctctctcatcctgccagctagcccagtttagtccacatcatcttgcaaagctcaaccagcccaagtcagccgggtccagctcattcatgtccaaaccagctcagtcatgctcatcctaactcagcctcaccatcatccacatcagctagcccagttcagctgagctcatcccagcccacttcaatcacagctcatttaagtacagctcaccccagctctatttagctcaagctagcttatttagcctacttcatcccagctcagcccagccaactcaactcatcctagctcagctaaaccctgctcagctcacccaagcaaagctgactccaacccagatcctttcagctcagctcacccagctcaggccagctcacccatcccagctcacccagcttagctcacccagcccagctcagcccagctcacccagcccagctcagcccagctcacccagcccagctcagcccagctcagctcagctcagctcagctcagctcagctcacccagctcagctcagccagctcagctcaccccagctcagtccagctcagttcagctcaccccagctcagctcacccaactcagctcactcaactcagctcacccaactcagctcagctcagttcacccagctcagctcacccagcccagcacagctcatctcacccagctcagctcacccagcccagctcaccccagctcaccccagctcagctcagctcaccccagctcagcccagctcagctcacccagctcagctcacccaactcagctcagctcagttcacccagctcagctcacccagcccagcacagctcatctcacccagcccagctcaccccagctcaccccagctcagctcagctcagcccagctcacccagctcagctcagctcaccccagctcagctcacccagctcagctcacccagcccagctcagctcagctcaccccagctcagcccagctcagctcacccagctcagctcacccagcccagctcaccccagctcaccccagctcagtccagctcagttcagctcacccagctcagctcacccaactcaactcagctcagttcacccagctcagctcagctcaccccagctcaccccagctcacccagctcagttcagctcaccccagctcagttcacccagctcagctcacccagcccagctcagcccagctcaccccagctcagctcaaccagatcagctcagcccagctcaccctagtttagttcacccagcccagctcaccccagctcagctcaccccaactcagctcacccagctcatcccagctcagccagctaatcccagctcagctcaccccagctcagctcacccagctcagctcacccaactcagctcaccccagctcaccccagctcatcccagctcatcccagttcagacctgttcagctcatctcaccccagctcagctcaccccagttcagctcacctagcccaactcaccccagctcagtccagctcagttcagctcaccccaactcatctcacccagctcagctcaccccagctcatcccagctcagctcaccccagttcagccctgttcagctcatctcacccagctcagctcatccagcccagctcaccccagctcaccccagctcagtccagctcagttcagctcacccagctcagctcacccaactcaactcagctcagttcacccagctcagctcagctcaccccagctcacccagctcagttcagctcaccccagctcagttcacccagctcagctcacccagcccagctcaaccagatcagctcagcccagctcaccctagtttagttcacccagcccagctcaccccagctcagctcaccccaactcagctcacctagctcatcccagctcagctcaccccagctcagctcaccccagctcatctcaccccagctcagctcacccagctcatcccagctcagctcagcccagctcatcccagccctgctcatcccagctcagctcagctcagcccagctcagcccagctcagcccagctcagcccagctcagcccagctcagctcaacccagctcagctcacccagcccagctcagcccagctcacccagctcagctcaccccagctcagctcaccccagctcatctcacccagctcagctcacccagctcagcccagctcagctcagctcacccagctcatctcacccagctcagctcaccccagctcatcccagctcagctcaccccagttcagccctgttcagctcatctcaccccagctcagctcacccagttcagctcatcccagcccatcccagctcagctcagcccagctcagcccagctcagcccagcccagcccagcccagctcagctcagcccagctcagcccagctcagtccagctcagcttagcccagcccagctcagctcagcccagctcagcccagctcagcccagctcagctcacccagctcaccccagctcagcccagctcagcccagctcagctcacccagctcaccccaccccagctcaccccagttcagcccagctcagcccagctcagcccagcccagcccagcccagcccagcccagctcagcccagctcagctcagcccagcccagctcagctcagcccagctcagcccagctcatcccagctcagctcaccccagctcagcccagctcagcccagctcagctcacccagctcaccccaccccagctcaccccagttcagcccagctcatccagctcagctcacccccagctctgctcacccagctcagctcagcttacccagctcagctcaactcacccagctcagctcacccagctcagctcagctcaccccagcccagctcagctcagctcaccccagctctgctcacccagctcagctcagctcacctcagctctgctcacccagctcagctcaaccacctcaggtcagcccagctcaccccagcttaccccagctcacccagctcagctcagctcacccagctcagctcacccagctcagctcaccccagcttaccccagctcaccccagctcagctaacccagctcagctcacccagctcagctcacccagctcagctcatcccagctcaccccagctaccacagagtagctcatgctagctcagctcaccccagcacaacacagcccaacacagctcagttcagagcagtccagtagagtttagctccaatcagcccagatcaagacaattcattccaatttggctatcttggttaagtcagcctagtttagcttagccggcctagctcaattcagctcattgcagtctacctcgttcctgctcaagtccagctttggctacctcagagtaatcatctcagcttagcacatttttgaagggctcagggaagcctacacatctcagtccaactgtgcttaactagagcctagcttcctagccaggctgtcaaccttgttcactaaattttgctcagcaagctt

SEQ ID NO: 2 - DNA, 22190 bp, synthetic construct
  misc_feature 1..22190: note = Tragetting Vector (long version)
  source 1..22190: mol_type = other DNA
gcggccgcaacctgggcaaatgggagcttagcaacaatgtagggggctggacctagacttcctacacatttgtagcagatgtgcagcttggtcttcatgtgtgtattaccctaacatttggagcaggagctgtctctgactctgttgcctgccattggatccccttcccctgcttgggctgccttgtttggccttagtaggaaaggatgtgcttagtcctgctgtgacttgatgtccctaggcagaatgataccccaggggggctccccatctctgaggagatgggcaaagggtaatggttggagggacttgtgaggctgggactgggaggagagaaaggagacagctgtaactggaatgatgttaagtgaacaaatgaatggatagattagatagacagatagacagacagacagacagacagacagacagacagacagacagatagaaagatagatagataaggggaaaaagaaacgtagctgagcaagccagagagagcaagccaaataagcagcattcctccatgacttttccttcagctcctgcctatgagtctgccttgacttccctcagtgattggttgtaagttaaaaggtgaaataaaccctttctttgacaagttgcttttggttctgatttttatcacagcaagagaaaatcaaactagaacaaacatgtatttttcctggcacatgtccatagtaaggcagaaatgatcttcagacctagaccatagatactacagagagcagaagtgtagataggtggacttactgtatgattgtaatccaagtaaatctacatagctagagagctagaggaaaggccaaagcttcctctgggaggtcagatcctgtcgcactgtagccaataaggcatattgcatcacaggaaaggactaagacccaggctggcaatagtgtctgtatcttaactagacctctctagtgagtgaggaaggaagtttgtgagagcccagactgtgggctcggaaggtacctgccatgcccctgttagtaactgagtactacagcaggagcaggtgttctctagaaagcctgagacaactctacttcttctctcaagagaccacctaatacaggcctgagagaacagactctggaaatagatgggacttaaggagctaagatctagagctcatctacagagcagaatcccagccaagagaacaaagaatactggctctctctcctgttccctactcctagagttctaaaacacactatagggaagggagcctctagacctccgtccattccccatcttgctcattccatcttcccatgtccccaggtctccaagccacagacactacctttcctattcacccacctttctgtgtccctaggtccccaggccatagtcacctccccccacacacaccccactcaccctgccccatctatgcccctagatgcttacttaccagagtcttttgtctgacgtggggctacaagcatctatgctccctaagcacctactgctgacctgtaggacccagctctgaaccaactcatataagtaaatacagactctcccctgtcttaggatggcctcctggatcaggaggagaccactgccaaagaaccttctctcagagcactgaactcctcccctgtaccacttaggacagacctgagacctattattactgattaccagagctctggcagtgaccacggaggagataggtccaccctggacacaggaaacacagcagcagagatactgctccatcacaacagtagagtgacactttagactttaatttgggtcactttcctgctgcagaggtgggatcagaaagcaaagagcagtatgagtgcctgataggcacccaagtacactatagagtactcatggtgaataaggtacctccatggcttcccagggaggggcactgccccacccccaccatcacagacctttctccatagttgataactcagacacaagtgaatgacagatggacctccatctactcttattttaaaaagaagacaaaccccacaggctcgagaactttagcgactgttttgagagaaatcattggtccctgactcaagagatgactggcagattggggatcagaatacccatactctgtggctagtgtgaggtttaagcctcagagtccctgtggtctctgactggtgcaaggttttgactaagcggagcaccacagtgctaactgggaccacggtgacacgtggctcaacaaaaaccttctgtttggagctctccaggggcagcctgagctatgaggaagtagagaggcttgagaaatctgaggaagaaaagagtagatctgagaggaaaggtagctttctggaggtcaggagacagtgcagagaagaacgagttactgtggacaggtcttagatggggaaagaatgagcaaatgcaagcatcagaagggtggatgcaatgtcctgccaaggacttaccaagaggatccccggacagagcaggcaggtggagttgactgagaggacagggtaggtgcaggtccctctctcgtttcctttctccttctcctgtttccttcctctcttgtcacaggtctcactatgctagccaaggctagcctgaaagattaccatcctacagatgggcccatccagttgagttaaggtggagatctctccaaacatctgagtttctgaggcttggatgccactggggacgccaagggactttgggctgggtttggttggccccagatgaagggctacttcactgggtctataattactctgatgtctaggaccagggggctcaggtcactcaggtcaggtgagtcctgcatctggggactgtggggttcaggtgtcctaaggcaggatgtggagagagttttagtataggaacagaggcagaacagagactgtgctactggtacttcgatgtctggggcgcagggaccacggtcaccgtctcctcaggtaagctggcttttttctttctgcacattccattctgaaatgggaaaagatattctcagatctccccatgtcaggccatctgccacactctgcatgctgcagaagcttttctgtaaggatagggtcttcactcccaggaaaagaggcagtcagaggctagctgcctgtggaacagtgacaatcatggaaaataggcatttacattgttaggctacatgggtagatgggtttttgtacacccactaaaggggtctatgatagtgtgactactttgactactggggccaaggcaccactctcacagtctcctcaggtgagtccttacaacctctctcttctattcagcttaaatagattttactgcatttgttgggggggaaatgtgtgtatctgaatttcaggtcatgaaggactagggacaccttgggagtcagaaagggtcattgggagccctggctgatgcagacagacatcctcagctcccagacttcatggccagagatttatagggatcctggccagcattgccgctaggtccctctcttctatgctttctttgtccctcactggcctccatctgagatcatcctggagccctagccaaggatcatttattgtcaggggtctaatcattgttgtcacaatgtgcctggtttgcttactggggccaagggactctggtcactgtctctgcaggtgagtcctaacttctcccattctaaatgcatgttggggggattctgagccttcaggaccaagattctctgcaaacgggaatcaagattcaacccctttgtcccaaagttgagacatgggtctgggtcagggactctctgcctgctggtctgtggtgacattagaactgaagtatgatgaaggatctgccagaactgaagcttgaagtctgaggcagaatcttgtccagggtctatcggactcttgtgagaattaggggctgacagttgatggtgacaatttcagggtcagtgactgtctggtttctctgaggtgaggctggaatataggtcaccttgaagactaaagaggggtccaggggcttctgcacaggcagggaacagaatgtggaacaatgacttgaatggttgattcttgtgtgacaccaggaattggcataatgtctgagttgcccaggggtgattctagtcagactctggggtttttgtcgggtatagaggaaaaatccactattgtgattactatgctatggactactggggtcaaggaacctcagtcaccgtctcctcaggtaagaatggcctctccaggtctttatttttaacctttgttatggagttttctgagcattgcagactaatcttggatatttgtccctgagggagccggctgagagaagttgggaaataaactgtctagggatctcagagcctttaggacagattatctccacatctttgaaaaactaagaatctgtgtgatggtgttggtggagtccctggatgatgggatagggactttggaggctcatttgagggagatgctaaaacaatcctatggctggagggatagttggggctacgcgtttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgctagtggatccggctggttctttccgcctcagaaggtacttttttttttttttttttttttttttttttttttttttttttttttttttttttttttttaaatttttgggaatttattgatttgcatttaaaagggaactgctgacaaagattcactggtaataatttgaacaagttggaaaatacagtcaacattactgaaacactactaaaataattccaggacagaacaaaacttcttagatgctgtctttgatgtgaaaattgactgcttcttacttttctaacacacggtggtataattaacaatattcaatcacttctattctttcctgcatatataaaaattaaaataccaattaaaaaactaatatatcttctctttatttcttacagatatgagttcaatgtttcactcaatagtgctgtggtttaagagaattttttcatttacaagttaaacaacaatccgcccaaagggaactgatagtctataggctcatagtgcaaataaacagtttaggaatgcagcaactgacatttctaaagtacaaaacagataaaattcttagaagatacatgcaaaaagctctactaagcagatggccacagaactagaacattgataattttactggcgatgtcaataggactccagatgtttccaaactcaacttgaactctcatcttaggctttgtattttgcttttccagtttcactaatgacacaaacatgattcaaatccctgaagtattcattatagtcaagggcatatcctacaacaaacttgtctggaatttcaaatccaacaaagtctggcttatatccaacacttcgtggggtccttttcaccagcaagcttgcgaccttgaccatctttggattatactgcctgaccaaggaaagcaaagtctgcattgttttgccagtgtcaattatatcttccacaatcaagacattctttccagttaaagttgagagatcatctccaccaattacttttatgtcccctgttgactggtcattacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtaacacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaatttcttggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacagtggatcccctcgagggacctaataacttcgtatagcatacattatacgaagttatattaagggttattgaatatgtcgactagacacactaatattaaaagtgtccaataacatttaaaactatactcatacgttaaaatataaatgtatatatgtacttttgcatatagtatacatgcatagccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatggggaataaacaccctttccaaatcctcagcataatgattaggtatgcaaaataaatcaaggtcataacctggttcatcatcactaatcacgacgccagggctgcgggtcgccataacggagccggccggcgcgcgggctgaataacttcgtataatgtgtactatacgaagttatttgttcaggaggaggaagccggtggcggagcagaggaggaggcggaggcgcagcaagacccccccccccctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaaagaattcgatatcaagcttatcgatgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaatttttgtcaatcaatttgaggtcttgtttgtgtagaactgacattacttaaagtttaaccgaggaatgggagtgaggctctctcataccctattcagaactgacttttaacaataataaattaagtttaaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaattcattttcaaaattaggttatgtaagaaattgaaggactttagtgtctttaatttctaatatatttagaaaacttcttaaaattactctattattcttccctctgattattggtctccattcaattcttttccaatacccgaagcatttacagtgactttgttcatgatcttttttagttgtttgttttgccttactattaagactttgacattctggtcaaaacggcttcacaaatctttttcaagaccactttctgagtattcattttaggagaaatactttttttttaaatgaatgcaattatctagacttatttcagttgaacatgctggttggtggttgagaggacactcagtcagtcagtgacgtgaagggcttctaagccagtccacatgctctgtgtgaactccctctggccctgcttattgttgaatgggccaaaggtctgagaccaggctgctgctgggtaggcctggactttgggtctcccacccagacctgggaatgtatggttgtggcttctgccacccatccacctggctgctcatggaccagccagcctcggtggctttgaaggaacaattccacacaaagactctggacctctccgaaaccaggcaccgcaaatggtaagccagaggcagccacagctgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgccagtaaatgtacttcctggttgttaaagaatggtatcaaaggacagtgcttagatccaaggtgagtgtgagaggacaggggctggggtatggatacgcagaaggaaggccacagctgtacagaattgagaaagaatagagacctgcagttgaggccagcaggtcggctggactaactctccagccacagtaatgacccagacagagaaagccagactcataaagcttgctgagcaaaatttagtgaacaaggttgacagcctggctaggaagctaggctctagttaagcacagttggactgagatgtgtaggcttccctgagcccttcaaaaatgtgctaagctgagatgattactctgaggtagccaaagctggacttgagcaggaacgaggtagactgcaatgagctgaattgagctaggccggctaagctaaactaggctgacttaaccaagatagccaaattggaatgaattgtcttgatctgggctgattggagctaaactctactggactgctctgaactgagctgtgttgggctgtgttgtgctggggtgagctgagctagcatgagctactctgtggtagctggggtgagctgggatgagctgagctgggtgagctgagctgggtgagctgagctgggttagctgagctggggtgagctggggtaagctggggtgagctgagctgggtgagctgagctgggtgagctgagctgagctgggtgagctggggtaagctggggtgagctgggctgacctgaggtggttgagctgagctgggtgagcagagctgaggtgagctgagctgagctgggtgagcagagctggggtgagctgagctgagctgggctggggtgagctgagctgagctgggtgagctgagctgggtgagttgagctgagctgggtaagctgagctgagctgggtgagcagagctgggggtgagctgagctggatgagctgggctgaactggggtgagctggggtggggtgagctgggtgagctgagctgggctgagctgggctgagctggggtgagctgagctgggatgagctgggctgagctgggctgagctgagctgggctgggctgagctgagctgggctgagctgggctgggctgggctgggctgggctgggctgagctgggctgagctgggctgaactggggtgagctggggtggggtgagctgggtgagctgagctgggctgagctgggctgagctggggtgagctgggtgagctgagctgggctgagctgggctgagctgggctgagctgagctgggctgggctaagctgagctggactgagctgggctgagctgggctgagctgagctgggctgggctgggctgggctgagctgggctgagctgggctgagctgagctgggatgggctgggatgagctgaactgggtgagctgagctggggtgagatgagctgaacagggctgaactggggtgagctgagctgggatgagctggggtgagctgagctgggtgagatgagctgggtgagctgagctgagctgggctgagctgggtgagctgagctgggtgagatgagctggggtgagctgagctggggtgagctgagctgggtgagctgggctgagctgggctgggtgagctgagctgggttgagctgagctgggctgagctgggctgagctgggctgagctgggctgagctgggctgagctgagctgagctgggatgagcagggctgggatgagctgggctgagctgagctgggatgagctgggtgagctgagctggggtgagatgagctggggtgagctgagctggggtgagctgagctgggatgagctaggtgagctgagttggggtgagctgagctggggtgagctgggctgggtgaactaaactagggtgagctgggctgagctgatctggttgagctgggctgggtgagctgagctgggtgaactgagctggggtgagctgaactgagctgggtgagctggggtgagctgagctgagctgggtgaactgagctgagttgagttgggtgagctgagctgggtgagctgaactgagctggactgagctggggtgagctggggtgagctgggctggatgagctgagctgggtgagatgagctgaacagggctgaactggggtgagctgagctgggatgagctggggtgagctgagctgggtgagatgagttggggtgagctgaactgagctggactgagctggggtgagttgggctaggtgagctgaactggggtgagctgagctggggtgagatgagctgaacaggtctgaactgggatgagctgggatgagctggggtgagctggggtgagctgagttgggtgagctgagctgggtgagctgagctggggtgagctgagctgggattagctggctgagctgggatgagctgggtgagctgagttggggtgagctgagctggggtgagctgggctgggtgaactaaactagggtgagctgggctgagctgatctggttgagctgagctggggtgagctgggctgagctgggctgggtgagctgagctgggtgaactgagctggggtgagctgaactgagctgggtgagctggggtgagctggggtgagctgagctgagctgggtgaactgagctgagttgagttgggtgagctgagctgggtgagctgaactgagctggactgagctggggtgagctggggtgagctgggctgggtgagctgagctgggtgagctgagctgggctgagctggggtgagctgagctgagctgggctgggtgagctgagctgggtgagctgagctggggtgagctgagctgagctgggtgagctgggctgagctgagctgagctggggtgagctggggtgagctgggctgggtgagatgagctgtgctgggctgggtgagctgagctgggtgaactgagctgagctgagttgggtgagctgagctgggtgagctgagctgggctgagctggggtgagctgagctgagctggggtgagctggggtgagctgggctgggtgagctgagctgggtgagatgagctgtgctgggctgggtgagctgagctgggtgaactgagctgagctgagttgggtgagctgagttgagtgagctgagttgggtgagctgagctggggtgagctgaactgagctggactgagctggggtgagctgagctggctgagctgagctgggtgagctgagctgagctgagctgagctgagctgagctgggctgagctgggctgggtgagctgggctgagctgggctgggtgagctgggctgagctgggctgggtgagctaagctgggtgagctgggatgggtgagctggcctgagctgggtgagctgagctgaaaggatctgggttggagtcagctttgcttgggtgagctgagcagggtttagctgagctaggatgagttgagttggctgggctgagctgggatgaagtaggctaaataagctagcttgagctaaatagagctggggtgagctgtacttaaatgagctgtgattgaagtgggctgggatgagctcagctgaactgggctagctgatgtggatgatggtgaggctgagttaggatgagcatgactgagctggtttggacatgaatgagctggacccggctgacttgggctggttgagctttgcaagatgatgtggactaaactgggctagctggcaggatgagagaggttgaactaggctcagatgagctaggctgagcagggcttaggtgaggtggtttgggatgagctgggataagttgggctcagatgagctaccatgaactgggctgaggtggcctgggagaacatgaatcgagttgcactgagtacagccaggatgaactgggataaactgggctaaactgggttgggctaagaatggactgcctgagttatgctaagctgagctgggatgaattggggattgtcaagacttaaatgagccagactgaactggttggacttgggtgagctgaggggacctgctgcagggccggcatagctgagttgaatttagatgaggaagggtgagctagactgagcaaggctagactgcttatgctgagctacactaacctggcctgagttgggccaggctttgttggccatgtctaaactgagttaagatcaacagggagtctagaggggtaggggatgaactaagatgaattacactagctgagctgaattgagataaggtatggtaacctgagctgaactggaaagagatgtactggataaccttaactgggctgagatgagctaggtctacccaggcctggatcagcttaattagggtaggctagaccaaactgtagcagtatgtattagcctgtgccaagctgggctacattaaactaaactggacttagctaggctcagattagtttcgctactctagatggggtaagttgggccaaactgggatgaactaatttaactagcctgagatgggcagatctgaatgagcagagctgggatgaactgaatgagtttcaccaggcctggaccagttaggctaggacctcgttctatagaggcagactgtgtgctacagtggagtttcaagatgattccatgagtcctccccgcccccaacataacccaccttcctcctaccctacaagcctgtctggtgtgtaaatcccagctttgtgtgctgatacagaagcctgagcccctcccccacctccacctacctattactttgggatgagaatagttctcccagccagtgtctcagagggaagccaagcaggacaggcccaaggctacttgagaagccaggatctaggcctctccctgagaacgggtgttcatgcccctagagttggctgaagggccagatccacctactctagaggcatctctccctgtctgtgaaggcttccaaagtcacgttcctgtggctagaaggcagctccatagccctgctgcagtttcgtcctgtataccaggttcacctactaccatatctagccctgcctgccttaagagtagcaacaaggaaatagcagggtgtagagggatctcctgtctgacaggaggcaagaagacagattcttacccctccatttctcttttatccctctctggtcctcagagagtcagtccttcccaaatgtcttccccctcgtctcctgcgagagccccctgtctgataagaatctggtggccatgggctgcctggcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacaaagatctgcatgtgcccattccaggtaagaaccaaaccctcccagcaggggtgcccaggcccaggcatggcccagagggagcagcggggtggggcttaggccaagctgagctcacaccttgacctttcattccagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtgagtggcctgggataagcccaatgcctagccctcccagattagggaagtcctcctacaattatggccaatgccacccagacatggtcatttgctccttgaactttggctccccagagtggccaaggacaagaatgagcaataggcagtagaggggtgagaatcagctggaaggaccagcatcttcccttaagtaggtttgggggatggagactaagcttttttccaacttcacaactagatatgtcataacctgacacagtgttctcttgactgcaggtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatggtaggtatccccccttcccttcccctccaattgcaggacccttcctgtacctcatagggagggcaggtcctcttccaccctatcctcactactgtcttcatttacagaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactggtaaacccacactgtacaatgtctccctgatcatgtctgacacaggcggcacctgctattgaccatgctagcgctcaaccaggcaggccctgggtgtccagttgctctgtgtatgcaaactaaccatgtcagagtgagatgttgcattttataaaaattagaaataaaaaaaatccattcaaacgtcactggttttgattatacaatgctcatgcctgctgagacagttgtgttttgcttgctctgcacacaccctgcatacttgcctccaccctggcccttcctctaccttgccagtttcctccttgtgtgtgaactcagtcaggcttacaacagacagagtatgaacatgcgattcctccagctacttctagatatatggctgaaagcttgcctaacctggtgcaggcagcattcaggcacatatatagacacacatgcatttatacatagatatataggtacacatgtgtagacacatacatgaatgtgtattcatggacacacagacaaaggtacacatatatacacatgagttcatgcgcacacacatgcatggacacttacaaacgccttcagagacaaataggcatagacacacaaccactcacagaaacagataccaatatgcatggtcctgtgtacacagaaacagactataggcaaatatacacaaataaactatatagatacaaagatatgcatatacacacatgtacagaaacatcttcacatgtgtacactaacatgtggacaggtatagcacacagatacacctggactctgaccagggctgtaatctccaaggctcacggctcagagagcctacactaggctgggtcactgatactcctcaggagcccactctatgattgggagagataaccccaggtacaaagtatgcctatctgtctcaacaccatggggcagaagatactccactaaccacccatgacagaaagttagccttggctgtgtctccattaatagaacacctcagaagaccaatgtgaaattgcctaacccactcacacccaccctgatctccagttcaaaatgcagaaaacataatgcagttgtccaaaagatgccccaaccacacacacacacacacacacacacacacacacacacacacacacacacatacacacacacacaccatcaaggagcctctgtaaggagtcaccacccaataacactgcctctttgggctcatatcctggacattcttcatattcatatccatttggggcctaggctttagatatccccaagggctcatctttacagggatcagagatcccaataaatgccctggtcccacagcctccctcaggtatctgtctgtttatctcttggtacctttcttagacgttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcg

SEQ ID NO: 3 - DNA, 14130 bp, synthetic construct
  misc_feature 1..14130: note = Targetting vector (short version)
  source 1..14130: mol_type = other DNA
gcggccgcaacctgggcaaatgggagcttagcaacaatgtagggggctggacctagacttcctacacatgtgtaacagatgtgcagcttggtcttcatgtgtgtattaccctaacatttggagcaggagctgtctctgactctgttgcctgccattggatccccttcccctgcttgggctgccttgtttggccttagtaggaaaggatgtgcttagtcctgctgtgacttgatgtccctaggcagaatgataccccaggggggctccccatctctgaggagatgggcaaagggtaatggttggagggacttgtgaggctgggactgggaggagagaaaggagacagctgtaactggaatgatgttaagtgaacaaatgaatggatagattagatagacagatagacagacagacagacagacagacagacagacagacagacagacagatagaaagatagatagataaggggaaaaagaaacgtagctgagcaagccagagagagcaagccaaataagcagcattcctccatgacttttccttcagctcctgcctatgagtctgccttgacttccctcagtgattggttgtaagttaaaaggtgaaataaaccctttctttgacaagttgcttttggttctgatttttatcacagcaagagaaaatcaaactagaacaaacatgtatttttcctggcacatgtccatagtaaggcagaaatgatcttcagacctagaccatagatactacagagagcagaagtgtagataggtggacttactgtatgattgtaatccaagtaaatctacatagctagagagctagaggaaaggccaaagcttcctctgggaggtcagatcctgtcgcactgtagccaataaggcatattgcatcacaggaaaggactaagacccaggctggcaatagtgtctgtatcttaactagatctctctagtgagtgaggaagtaaatttgtgagagcccagactgtgggctcggaaggtacctgccatgcccctgttagtaactgagtactacagcaggagcaggtgttctctagaaagcctgagacaactctacttcttctctcaagagaccacctaatacaggcctgagagaacagactctggaaatagatgggacttacggagctaagatctagagctcatctacagagcagaatcccagccaagagaacaaagaatactgactctctcctgttccctactcctagagttctaaaacacactatagggaagggagcctctagacctccgtccattccccatcttgctcattccatcttcccatgtccccaggtctccaagccacagacaccacctttcctattcacccacctttctgtgtccctaggtccccaggccatagtcacctccccccacaccccgctcaccctgccccatctatgcccctagatgcttacttaccagagtcttttgtctgacgtggggctacaagcatctatgctccctaagcacctactgctgacctgtaggacccagctctgaaccaactcatataagtaaatacagactctcccctgtcttaggatggccccctgggtcaggaggagaccactgccaaggaaccttctcttagagcactgaactcctcccctgtaccacttaggacagacctgagacctattattactgattaccagagctctggcagtgaccacggaggagatagatccaccctggacacaggaaacacagcaccagagatactgcttcatcacaacagtagagtgacactttagactttaatttgggtcactttcctgctgtagaggtgggatcagaaagcaaagagcagtatgagtgcctgataggcacccaagtacactatagagtactcatggtgaataaggtacctccatggcttcccagggaggggcactgccccacccccaccatcacagacctttctccatagttgataactcagacacaagtgaatgacagatggacctccatctgctcttattttaaaaagaagacaaaccccacaggctcgagaactttagcgactgttttgagagaaatcattggtccctgactcaagagatgactggcagattggggatcagaatacccatactctgtggctagtgtgaggtttaagcctcagagtccctgtggtctctgactggtgcaaggttttgactaagcggagcaccacagtgctaactgggaccacggtgacacgtggctcaacaaaaaccttctgtttggagctctccaggggcagcctgagctatgaggaagtagagaggcttgagaaatctgaggaagaaaagagtagatctgagaggaaaggtagctttctggaggtcaggagacagtgcagagaagaacgagttactgtggacaggtcttagatggggaaagaatgagcaaatgcaagcatcagaagggtggatgcaatgtcctgccaaggacttaccaagaggatccccggacagagcaggcaggtggagttgactgagaggacaggataggtgcaggtccctctcttgtttcctttctccttctcctgtttccttcttctcttgtcacaggtctcactatgctagccaaggctagcctgaaagattaccatcctacagatgggcccatccagttgaattaaggtggagatctctccaaacatctgagtttctgaggcttggatgccactggggacgccaagggactttgggatgggtttggttggccccagatgaagggctacttcactgggtctataattactctgatgtctaggaccagggggctcaggtcactcaggtcaggtgagtcctgcatctggggactgtggggttcaggtggcctaaggcaggatgtggagagagttttagtataggaacagaggcagaacagagactgtgctactggtacttcgatgtctggggcacagggaccacggtcaccgtctcctcaggtaagctggcttttttctttctgcacattccattctgaaacgggaaaagatattctcagatctccccatgtcaggccatctgccacactctgcatgctgcagaagcttttctgtaaggatagggtcttcactcccaggaaaagaggcagtcagaggctagctgcctgtggaacagtgacaatcatggaaaataggcatttacattgttaggctacatgggtagatgggtttttgtacacccactaaaggggtctatgatagtgtgactactttgactactggggccaaggcaccactctcacagtctcctcaggtgagtccttacaacctctctcttctattcagcttaaatagattttactgcatttgttgggggggaaatgtgtgtatctgaatttcaggtcatgaaggactagggacaccttgggagtcagaaagggtcattgggagccctggctgacgcagacagacatcctcagctcccatacttcatggccagagatttatagggatcctggccagcattgccgctaggtccctctcttctatgctttctttgtccctcactggcctccatctgagatcatcctggagccctagccaaggatcatttattgtcaggggtctaatcattgttgtcacaatgtgcctggtttgcttactggggccaagggactctggtcactgtctctgcaggtgagtcctaacttctcccattctaaatgcatgttggggggattctgggccttcaggaccaagattctctgcaaacgggaatcaagattcaacccctttgtcccaaagttgagacatgggtctgggtcagggactctctgcctgctggtctgtggtgacattagaactgaagtatgatgaaggatctgccagaactgaagcttgaagtctgaggcagaatcttgtccagggtctatcggactcttgtgagaattaggggctgacagttgatggtgacaatttcagggtcagtgactgtctggtttctctgaggtgaggctggaatataggtcaccttgaagactaaagaggggtccaggggcttctgcacaggcagggaacagaatgtggaacaatgacttgaatggttgattcttgtgtgacaccaggaattggcataatgtctgagttgcccaggggtgattctagtcagactctggggtttttgtcgggtatagaggaaaaatccactattgtgattactatgctatggactactggggtcaaggaacctcagtcaccgtctcctcaggtaagaatggcctctccaggtctttatttttaacctttgttatggagttttctgagcattgcagactaatcttggatatttgtccctgagggagccggctgagagaagttgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctacgcgtttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgctagtggatcctggttctttccgcctcagaaggtacttttttttttttttttttttttttttttttttttttttttttttttttttttttttttttaaatttttgggaatttattgatttgcatttaaaagggaactgctgacaaagattcactggtaataatttgaacaagttggaaaatacagtcaacattactgaaacactactaaaataattccaggacagaacaaaacttcttagatgctgtctttgatgtgaaaattgactgcttcttacttttctaacacacggtggtataattaacaatattcaatcacttctattctttcctgcatatataaaaattaaaataccaattaaaaaactaatatatcttctctttatttcttacagatatgagttcaatgtttcactcaatagtgctgtggtttaagagaattttttcatttacaagttaaacaacaatccgcccaaagggaactgatagtctataggctcatagtgcaaataaacagtttaggaatgcagcaactgacatttctaaagtacaaaacagataaaattcttagaagatacatgcaaaaagctctactaagcagatggccacagaactagaacattgataattttactggcgatgtcaataggactccagatgtttccaaactcaacttgaactctcatcttaggctttgtattttgcttttccagtttcactaatgacacaaacatgattcaaatccctgaagtattcattatagtcaagggcatatcctacaacaaacttgtctggaatttcaaatccaacaaagtctggcttatatccaacacttcgtggggtccttttcaccagcaagcttgcgaccttgaccatctttggattatactgcctgaccaaggaaagcaaagtctgcattgttttgccagtgtcaattatatcttccacaatcaagacattctttccagttaaagttgagagatcatctccaccaattacttttatgtcccctgttgactggtcattacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtaacacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaatttcttggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacagtggatcccctcgagggacctaataacttcgtatagcatacattatacgaagttatattaagggttattgaatatgtcgactagacacactaatattaaaagtgtccaataacatttaaaactatactcatacgttaaaatataaatgtatatatgtacttttgcatatagtatacatgcatagccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatggggaataaacaccctttccaaatcctcagcataatgattaggtatgcaaaataaatcaaggtcataacctggttcatcatcactaatcacgacgccagggctgcgggtcgccataacggagccggccggcgcgcgggctgaataacttcgtataatgtgtactatacgaagttatttgttcaggaggaggaagccggtggcggagcagaggaggaggcggaggcgcagcaagacccccccccccctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagccaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaaagaattcgtagttggagattttcagtttttagaataaaagtattagctgcggaatatacttcaggaccacctctgtgacagcatttatacagtatccgatgcatagggacaaagagtggagtggggcactttctttagatttgtgaggaatgttccacactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaatttttgtcaatcaatttgaggtcttgtttgtgtagaactgacattacttaaagtttaaccgaggaatgggagtgaggctctctcataccctattcagaactgacttttaacaataataaattaagtttaaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaattcattttcaaaattaggttatgtaagaaattgaaggactttagtgtctttaatttctaatatatttagaaaacttcttaaaattactctattattcttccctctgattattggtctccattcaattcttttccaatacccgaagcatttacagtgactttgttcatgatcttttttagttgtttgttttgccttactattaagactttgacattctggtcaaaacggcttcacaaatctttttcaagaccactttctgagtattcattttaggagaaatactttttttttaaatgaatgcaattatctagacttatttcggttgaacatgctggttggtggttgagaggacactcagtcagtcagtggcgtgaagggcttctaagccagtccacatgctctgtgtgaactccctctggccctgcttattgttgaatgggccaaaggtctgagaccaggctgctgctgggtaggcctggactttgggtctcccacccagacctgggaatgtatggttgtggcttctgccacccatccacctggctgctcatggaccagccagcctcggtggctttgaaggaacaattccacacaaagactctggacctctccgaaaccaggcaccgcaaatggtaagccagaggcagccacagctgtggctgctgctcttaaagcttgtaaactgtttctgcttaagagggactgagtcttcagtcattgctttagggggagaaagagacatttgtgtgtcttttgagtaccgttgtctgggtcactcacatttaactttccttgaaaaactagtaaaagaaaaatgttgcctgttaaccaataatcatagagctcatggtattttgaggaaatcttagaaaacgtgtatacaattgtctggaattatttcagttaagtgtattagttgaggtactgatgctgtctctacttcagttatacatgtgggtttgaattttgaatctattctggctcttcttaagcagaaaatttagataaaatggatacctcagtggtttttaatggtgggtttaatatagaaggaatttaaattggaagctaatttagaatcagtaaggagggacccaggctaagaaggcaatcctgggattctggaagaaaagatgtttttagtttttatagaaaacactactacattcttgatctacaactcaatgtggtttaatgaatttgaagttgccagtaaatgtacttcctggttgttaaagaatggtatcaaaggacagtgcttagatccaaggtgagtgtgagaggacaggggctggggtatggatacgcagaaggaaggccacagctgtacagaattgagaaagaatagagacctgcagttgaggccagcaggtcggctggactaactctccagccacagtaatgacccagacagagaaggccagactcataaagctttatcgataccgtcgacctcgagggggggcccggtacctttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcg

SEQ ID NO: 4 - DNA, 3551 bp, Mus musculus
  source 1..3551: mol_type = genomic DNA
aagcttgctgagcaaaattaagggaacaaggttgagagccctagtaagcgaggctctaaaaagcatggctgagctgagatgggtgggcttctctgagcgcttctaaaatgcgctaaactgaggtgattactctgaggtaagcaaagctgggcttgagccaaaatgaagtagactgtaatgaactggaatgagctgggccgctaagctaaactaggctggcttaaccgagatgagccaaactggaatgaacttcattaatctaggttgaatagagctaaactctactgcctacactggactgttctgagctgagatgagctggggtgagctcagctatgctacgctgtgttggggtgagctgatctgaaatgagctactctggagtagctgagatggggtgagatggggtgagctgagctgggctgagctggactgagctgagctagggtgagctgagctgggtgagctgagctaagctggggtgagctgagctgagcttgactgagctagggtgagctggactgagctggggtgagctgagctgagctggggtaagctgggatgagctggggtgagctgagctgagctggagtgagctgagctgggctgagctggggtgagctgggctgggctgagctggggtgagctgggctgagctggggtgagctgagctggggtgagctgagctgagctggggtgagctgagctgagctggggtgagctgagctggggtgagctgagctgagctgggctgagctgaggtgagctgagctggggtgagctgagctggggtgagctgagctgagctggggtaagctgggatgagctggggtgagctgagctgagctggagtgagctgagctgggctgagctgggctgagctggggtgagctgagctggggtgagctgagctgagctgggctgagctgaggtgagctgagctggggtgagctgagctgagctggggtgagctgagctgagctggggtgagctgagctggggtgagctgagctggggtgagctgagctgagctggggtgagctgagctggggtgagctgagctgagctggggtgagctgagctgagctggggtgagctgagctgagctgagctggggtgagctgagctgagctggggtgagctgagctgagctggggtgagctgagctggggtgagctgggctgagctgagctgggctgagctgagctgagctgagctgagctggggtgagctgagctgggctgagctggggtgagctgggctgagctggggtgagctgagctggggtgagctgagctgagctggggtgagctgagctgagctggggtgagctgagctggggtgagctgagctgagctgggctgagctgagctgagctggggtgagctgagctgagctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagctgagctgagctgagctgagctgagctggggtgagctggggtgagctgagctggggtgagctgagctgagctggggtgagctgagctgagctgagctgagctgagctgagctgggtgagctgagctgagctgagctggggtgagctgagctggggtgagctgagctgagctggggtgagctgagctggggtgagctgagctgagctggggtgagctggggtgagctggggtgagctggggtgagctgagctgaactggggtgagctgggctgagctggggtgagctgagctgagctgggctgagctggggtgagctggggtgagctggggtgagctgagctgagctagggtgagctgagctgagctagggtgagctgagctgagctggggtgagctgagctgagctggggtgagctgagctgagctggggtgagctgagctgagctggggtgagctgagctgagctggggtgagcttggctgagctggggtgagctggggtgagctgagctggggtgagctggggtaagctgagctgagctggggtgagctgagctgagctggggtgagctggggtgagctgagctgagctgagctgggtgatctgagctgagctgagctgggtgagctgagctgagctgagctgggtgagctgagctgagctgagctgagctgggtgagctgagctgagctgagctgagctgagctgagctggggtgagctgggctgagctgagctgagctggggtgagctgagctgagctgagctgagctggggtgagctgggctgagctggggtgagctgggctgagctgagctgggtgagctgagctgaactgagctgagctgggtgagctgagctgagctgagctgggtgagctgagctgggctgagctgagctgggtgagctgagctgaactgagctgagctgggtgagctgagctgagctgagctgggtgagctgagctggggtgagctgagctgagctggggtgagctgagctgagctgagctgggtgagctgagctggggtgagctgagctgagctggggtgagctgagctgagctggggtgagctgagctgagctggggtgagctgagctgagctggggtgagctgagctagggtgaactgggctgggtgagctggagtgagctgagctgaggtgaactggggtgagccgggatgttttgagttgagctggggtaagatgagctgaactggggtaagatgggatgagctgtggtgaggggagctggattgaactgagctgtgtgagctgagctggggtcagctgagcaagagtgagtagagctggctggccagaaccagaatcaattaggctaagtgagccagattgcgctgggatcagctgtactcagatgagctgggatgaggtaggctgggatgagctgggctagctgacatggattatgtgaggctgagctagcatgggctggcctagctgatgagctaagcttgaatgaacggggctgagctggactcagatgtgctagactgagctgtactggatgatctggtgtagggtgatctggactcaactgggctggctgatgggatgccccaggttgaactaggctcagataagttaggctgagtagggcctggttgagatggttcgggatgagctgggaaaagatggactgggaccatgaactgggctgagctgggttgggagaccatgaattgagctgaactgagtgcagctgggataaactgggttgagctaagaatagactacctgaattgtgccaaactgggctgggatcaattggaaattatcaggatttagatgagccggactaaactatgctgagctggactggttggatgtgttgaactggcctgctgctgggctggcatagctgagttgaacttaaatgaggaaggatgagcaaggctagcctgcttgcatagagctgaactttagcctagcctgagctggaccagcctgagctgagtaggtctaaactgagttaaaaatcaacagggataatttaacagctaatttaacaagcctgaggtctgagatt

SEQ ID NO: 5 - DNA, 2484 bp, Mus musculus
  source 1..2484: mol_type = genomic DNA
aagcttgctgagcaaaattaagggaacaaggttgagagccctagtaagcgaggctctaaaaagcacagctgagctgagatgggtgggcttctctgagtgcttctaaaatgcgctaaactgaggtgattactctgaggtaagcaaagctgggcttgagccaaaatgaagtagactgtaatgaactggaatgagctgggccgctaagctaaactaggctggcttaaccgagatgagccaaactggaatgaacttcattaatctaggttgaatagagctaaactctactgcctacactggactgttctgagctgagatgagctggggtgagctcagctatgctacgctgtgttggggtgagctgatctgaaatgagatactctggagtagctgagatggggtgagatggggtgagctgagctgggctgagctagactgagctgagctagggtgagctgagctgggtgagctgagctaagctggggtgagctgagctgagcttggctgagctagggtgagctgggctgagctggggtgagctgagctgagctggggtaagctgggatgagctggggtgagctgagctgagctggagtgagctgagctgggctgagctggggtgagctgggctgagctgggctgagctgggctgagctggggtgagctgagctggggtgagctgagctgagctggggtgagctgagctgagctggggtgagctggggtgagctgagctggggtgagctgagctgagctggggtgagctgagctggggtgagctgagctgagctggggtgagctgagctgagctgagctgagctgagctggggtgagctgagctgagctgagctggggtgagctggggtgagctgagctgagctggagtgagctgagctgggctgagctggggtgagctgggctgagctggggtgagctgagctgagctgagctgagctggggtgagctgagctgagctggggtgagctgagctggggtgagctgggctgagctgagctgagctgagctgagctgagctgagctgagctgagctgagctgagctgagctgagctgagctgagctgagctgagctggggtgagctgagctgagctgggctgagctggggtgagctgggctgagctgggctgagctgggctgagctggggtgagctgagctggggtgagctgagctgagctgggctgagctgagctgagctggggtgagctgagctgagctggggtgagctgagctgagctgagctggggtgagctgagctgggctgagcagggctgagctggggtgagctgagctgagctggggtgagctgggctgagctgggctgagctgagctgagctgggctgagctgggctgagctgggctgagctgggctgagctgggctgagctggggtgagctgagctgagctggggtgagctggggtgagctgagctggggtgagctgagctggggtgagctgagctgagctggggtgagctgagctggggtgagctgagctgagctggggtgagctgagctgagctggggtgagctgagctagggtgaactgggctgggtgagctggagtgagctgagctgaggtgaactggggtgagccgggatgttttgagttgagctggggtaagatgagctgaactggggtaaactgggatgagctgtggtgagcggagctggattgaactgagctgtgtgagctgagctggggtcagctgagcaagagtgagtagagctggctggccagaaccagaatcaattaggctaagtgagccagattgtgctgggatcagctgtactcagatgagctgggatgaggtaggctgggatgagctgggctagctgacatggattatgtgaggctgagctagcatgggctggcctagctgatgagctaagcttgaatgagcggggctgagctggactcagatgtgctagactgagctgtactggatgatctggtgtagggtgatctggactcaactgggctggctgatgggatgcgccaggttgaactaggctcagataagttaggctgagtagggcctggttgagatggttcgggatgagctgggaaaagatggactcggaccatgaactgggctgagctgggttgggagaccatgaattgagctgaactgagtgcagctgggataaactgggttgagctaagaatagactacctgaattgtgccaaactcggctgggatcaattggaaattatcaggatttagatgagccggactaaactatgctgagctggactggttggatgtgttgaactggcctgctgctgggctggcatagctgagttgaacttaaatgaggaaggctgagcaaggctagcctgcttgcatagagctgaactttagcctagcctgagctggaccagcctgagctgagtaggtctaaactgagttaaaaatcaacagggataatttaacagctaatttaacaagcctgaggtctgagatt

SEQ ID NO: 6 - DNA, 4515 bp, synthetic construct
  misc_feature 1..4515: note = 5' homology arm of targetting vector
  source 1..4515: mol_type = other DNA
aacctgggcaaatgggagcttagcaacaatgtagggggctggacctagacttcctacacatgtgtaacagatgtgcagcttggtcttcatgtgtgtattaccctaacatttggagcaggagctgtctctgactctgttgcctgccattggatccccttcccctgcttgggctgccttgtttggccttagtaggaaaggatgtgcttagtcctgctgtgacttgatgtccctaggcagaatgataccccaggggggctccccatctctgaggagatgggcaaagggtaatggttggagggacttgtgaggctgggactgggaggagagaaaggagacagctgtaactggaatgatgttaagtgaacaaatgaatggatagattagatagacagatagacagacagacagacagacagacagacagacagacagacagacagatagaaagatagatagataaggggaaaaagaaacgtagctgagcaagccagagagagcaagccaaataagcagcattcctccatgacttttccttcagctcctgcctatgagtctgccttgacttccctcagtgattggttgtaagttaaaaggtgaaataaaccctttctttgacaagttgcttttggttctgatttttatcacagcaagagaaaatcaaactagaacaaacatgtatttttcctggcacatgtccatagtaaggcagaaatgatcttcagacctagaccatagatactacagagagcagaagtgtagataggtggacttactgtatgattgtaatccaagtaaatctacatagctagagagctagaggaaaggccaaagcttcctctgggaggtcagatcctgtcgcactgtagccaataaggcatattgcatcacaggaaaggactaagacccaggctggcaatagtgtctgtatcttaactagatctctctagtgagtgaggaagtaaatttgtgagagcccagactgtgggctcggaaggtacctgccatgcccctgttagtaactgagtactacagcaggagcaggtgttctctagaaagcctgagacaactctacttcttctctcaagagaccacctaatacaggcctgagagaacagactctggaaatagatgggacttacggagctaagatctagagctcatctacagagcagaatcccagccaagagaacaaagaatactgactctctcctgttccctactcctagagttctaaaacacactatagggaagggagcctctagacctccgtccattccccatcttgctcattccatcttcccatgtccccaggtctccaagccacagacaccacctttcctattcacccacctttctgtgtccctaggtccccaggccatagtcacctccccccacaccccgctcaccctgccccatctatgcccctagatgcttacttaccagagtcttttgtctgacgtggggctacaagcatctatgctccctaagcacctactgctgacctgtaggacccagctctgaaccaactcatataagtaaatacagactctcccctgtcttaggatggccccctgggtcaggaggagaccactgccaaggaaccttctcttagagcactgaactcctcccctgtaccacttaggacagacctgagacctattattactgattaccagagctctggcagtgaccacggaggagatagatccaccctggacacaggaaacacagcaccagagatactgcttcatcacaacagtagagtgacactttagactttaatttgggtcactttcctgctgtagaggtgggatcagaaagcaaagagcagtatgagtgcctgataggcacccaagtacactatagagtactcatggtgaataaggtacctccatggcttcccagggaggggcactgccccacccccaccatcacagacctttctccatagttgataactcagacacaagtgaatgacagatggacctccatctgctcttattttaaaaagaagacaaaccccacaggctcgagaactttagcgactgttttgagagaaatcattggtccctgactcaagagatgactggcagattggggatcagaatacccatactctgtggctagtgtgaggtttaagcctcagagtccctgtggtctctgactggtgcaaggttttgactaagcggagcaccacagtgctaactgggaccacggtgacacgtggctcaacaaaaaccttctgtttggagctctccaggggcagcctgagctatgaggaagtagagaggcttgagaaatctgaggaagaaaagagtagatctgagaggaaaggtagctttctggaggtcaggagacagtgcagagaagaacgagttactgtggacaggtcttagatggggaaagaatgagcaaatgcaagcatcagaagggtggatgcaatgtcctgccaaggacttaccaagaggatccccggacagagcaggcaggtggagttgactgagaggacaggataggtgcaggtccctctcttgtttcctttctccttctcctgtttccttcttctcttgtcacaggtctcactatgctagccaaggctagcctgaaagattaccatcctacagatgggcccatccagttgaattaaggtggagatctctccaaacatctgagtttctgaggcttggatgccactggggacgccaagggactttgggatgggtttggttggccccagatgaagggctacttcactgggtctataattactctgatgtctaggaccagggggctcaggtcactcaggtcaggtgagtcctgcatctggggactgtggggttcaggtggcctaaggcaggatgtggagagagttttagtataggaacagaggcagaacagagactgtgctactggtacttcgatgtctggggcacagggaccacggtcaccgtctcctcaggtaagctggcttttttctttctgcacattccattctgaaacgggaaaagatattctcagatctccccatgtcaggccatctgccacactctgcatgctgcagaagcttttctgtaaggatagggtcttcactcccaggaaaagaggcagtcagaggctagctgcctgtggaacagtgacaatcatggaaaataggcatttacattgttaggctacatgggtagatgggtttttgtacacccactaaaggggtctatgatagtgtgactactttgactactggggccaaggcaccactctcacagtctcctcaggtgagtccttacaacctctctcttctattcagcttaaatagattttactgcatttgttgggggggaaatgtgtgtatctgaatttcaggtcatgaaggactagggacaccttgggagtcagaaagggtcattgggagccctggctgacgcagacagacatcctcagctcccatacttcatggccagagatttatagggatcctggccagcattgccgctaggtccctctcttctatgctttctttgtccctcactggcctccatctgagatcatcctggagccctagccaaggatcatttattgtcaggggtctaatcattgttgtcacaatgtgcctggtttgcttactggggccaagggactctggtcactgtctctgcaggtgagtcctaacttctcccattctaaatgcatgttggggggattctgggccttcaggaccaagattctctgcaaacgggaatcaagattcaacccctttgtcccaaagttgagacatgggtctgggtcagggactctctgcctgctggtctgtggtgacattagaactgaagtatgatgaaggatctgccagaactgaagcttgaagtctgaggcagaatcttgtccagggtctatcggactcttgtgagaattaggggctgacagttgatggtgacaatttcagggtcagtgactgtctggtttctctgaggtgaggctggaatataggtcaccttgaagactaaagaggggtccaggggcttctgcacaggcagggaacagaatgtggaacaatgacttgaatggttgattcttgtgtgacaccaggaattggcataatgtctgagttgcccaggggtgattctagtcagactctggggtttttgtcgggtatagaggaaaaatccactattgtgattactatgctatggactactggggtcaaggaacctcagtcaccgtctcctcaggtaagaatggcctctccaggtctttatttttaacctttgttatggagttttctgagcattgcagactaatcttggatatttgtccctgagggagccggctgagagaagttgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggct

SEQ ID NO: 7 - DNA, 25 bp, synthetic construct
  misc_feature 1..25: note = Oligonucleotide HV2-5
  source 1..25: mol_type = other DNA
agatcaccttgaaggagtctggtcc

SEQ ID NO: 8 - DNA, 25 bp, synthetic construct
  misc_feature 1..25: note = Oligonucleotide HV4-4
  source 1..25: mol_type = other DNA
tggtgaagccttcggagaccctgtc

SEQ ID NO: 9 - DNA, 25 bp, synthetic construct
  misc_feature 1..25: note = Oligonucleotide HV1-3
  source 1..25: mol_type = other DNA
cactagctatgctatgcattgggtg

SEQ ID NO: 10 - DNA, 25 bp, synthetic construct
  misc_feature 1..25: note = Oligonucleotide HV1-2
  source 1..25: mol_type = other DNA
atggatcaaccctaacagtggtggc

SEQ ID NO: 11 - DNA, 25 bp, synthetic construct
  misc_feature 1..25: note = Oligonucleotide HV6-1
  source 1..25: mol_type = other DNA
ggaaggacatactacaggtccaagt

SEQ ID NO: 12 - DNA, 25 bp, synthetic construct
  misc_feature 1..25: note = Oligonucleotide C
  source 1..25: mol_type = other DNA
taggtacttgccccctgtcctcagt

SEQ ID NO: 13 - DNA, 25 bp, synthetic construct
  misc_feature 1..25: note = Oligonucleotide KV1-9
  source 1..25: mol_type = other DNA
agcccagtgtgttccgtacagcctg

SEQ ID NO: 14 - DNA, 25 bp, synthetic construct
  misc_feature 1..25: note = Oligonucleotide KV1-8
  source 1..25: mol_type = other DNA
atcctcattctctgcatctacagga

SEQ ID NO: 15 - DNA, 25 bp, synthetic construct
  misc_feature 1..25: note = Oligonucleotide KV1-6
  source 1..25: mol_type = other DNA
ggtaaggatggagaacactggcagt

SEQ ID NO: 16 - DNA, 25 bp, synthetic construct
  misc_feature 1..25: note = Oligonucleotide KV1-5
  source 1..25: mol_type = other DNA
ttagtagctggttggcctggtatca

SEQ ID NO: 17 - DNA, 25 bp, synthetic construct
  misc_feature 1..25: note = Oligonucleotide Ck
  source 1..25: mol_type = other DNA
ctttgctgtcctgatcagtccaact

SEQ ID NO: 18 - DNA, 10 bp, synthetic construct
  misc_feature 1..10: note = Rat Switch
  repeat_region 1..10: note = Repeated 148 times
  source 1..10: mol_type = other DNA
gagctgagct

SEQ ID NO: 19 - DNA, 10 bp, synthetic construct
  misc_feature 1..10: note = Rat Switch
  repeat_region 1..10: note = Repeat 25 times
  source 1..10: mol_type = other DNA
ggggtggggt

SEQ ID NO: 20 - DNA, 10 bp, synthetic construct
  misc_feature 1..10: note = Rat Switch
  repeat_region 1..5: note = Repeat 82 times
  source 1..10: mol_type = other DNA
gggctgggct

SEQ ID NO: 21 - protein, 6 aa, synthetic construct
  REGION 1..6: note = Antibody Sequence
  VAR_SEQ 1..2: note = Xaa in position 1 is P or R. If P, then Xaa in position 2 is R or W
  VAR_SEQ 1..2: note = If Xaa in position 1 is R, then Xaa in position 2 is T
XXTFGQ

SEQ ID NO: 22 - protein, 17 aa, synthetic construct
  REGION 1..17: note = Antibody Sequence
  VAR_SEQ 1..2: note = Xaa in position 1 is P or R. If P, then Xaa in position 2 is R or W
  VAR_SEQ 1..2: note = If Xaa in position 1 is R, then Xaa in position 2 is T
XXTFGQGTKVEIKRADA

SEQ ID NO: 23 - protein, 6 aa, synthetic construct
  REGION 1..6: note = Antibody Sequence
  VAR_SEQ 1..2: note = Xaa in position 1 is P or R. If P, then Xaa in position 2 is R or W
  VAR_SEQ 1..2: note = If Xaa in position 1 is R, then Xaa in position 2 is T
XXTFGQ

SEQ ID NO: 24 - protein, 17 aa, synthetic construct
  REGION 1..17: note = Antibody Sequence
  VAR_SEQ 2: note = Xaa in position 2 is R or W
  VAR_SEQ 1: note = Xaa in position 1 is P
XXTFGQGTKVEIKRADA

SEQ ID NO: 25 - DNA, 24 bp, synthetic construct
  misc_feature 1..24: note = Primer E1554
  source 1..24: mol_type = other DNA
atgacttcagtgttgttctggtag

SEQ ID NO: 26 - DNA, 19 bp, synthetic construct
  misc_feature 1..19: note = Primer E1555
  source 1..19: mol_type = other DNA
caccagattcttatcagac

SEQ ID NO: 27 - DNA, 35 bp, synthetic construct
  misc_feature 1..35: note = ELP1352_Cy1
  source 1..35: mol_type = other DNA
agagcggccgctgggcaacgttgcaggtgacggtc

SEQ ID NO: 28 - DNA, 34 bp, synthetic construct
  misc_feature 1..34: note = ELP1353_Cy2b
  source 1..34: mol_type = other DNA
agagcggccgctttgtccaccgtggtgctgctgg

SEQ ID NO: 29 - DNA, 33 bp, synthetic construct
  misc_feature 1..33: note = ELP1354_Cy2a
  source 1..33: mol_type = other DNA
agagcggccgcacattgcaggtgatggactggc

SEQ ID NO: 30 - DNA, 35 bp, synthetic construct
  misc_feature 1..35: note = ELP1356_VH4-4
  source 1..35: mol_type = other DNA
aggacgcgtgaaacacctgtggttcttcctcctgc

SEQ ID NO: 31 - DNA, 33 bp, synthetic construct
  misc_feature 1..33: note = ELP1357_VH1-2,3
  source 1..33: mol_type = other DNA
aggacgcgtcaccatggactggacctggaggat

SEQ ID NO: 32 - DNA, 34 bp, synthetic construct
  misc_feature 1..34: note = ELP1358_VH6-1
  source 1..34: mol_type = other DNA
aggacgcgtatgtctgtctccttcctcatcttcc

SEQ ID NO: 33 - DNA, 21 bp, synthetic construct
  misc_feature 1..21: note = mIgG1_2 rev
  source 1..21: mol_type = other DNA
ggggccagtggatagacagat

SEQ ID NO: 34 - DNA, 18 bp, synthetic construct
  misc_feature 1..18: note = mIgG2b rev
  source 1..18: mol_type = other DNA
cagtggatagactgatgg

SEQ ID NO: 35 - DNA, 18 bp, synthetic construct
  misc_feature 1..18: note = mIgG2a_2 rev
  source 1..18: mol_type = other DNA
cagtggatagaccgatgg

SEQ ID NO: 36 - DNA, 20 bp, synthetic construct
  misc_difference 1..20: note = mCH1 unirev
  source 1..20: mol_type = other DNA
kcaggggccagtggatagac

SEQ ID NO: 37 - DNA, 20 bp, synthetic construct
  misc_difference 1..20: note = mCH1 unirev_2
  source 1..20: mol_type = other DNA
tarccyttgacmaggcatcc

SEQ ID NO: 38 - protein, 24 aa, synthetic construct
  REGION 1..24: note = 16C9
QEVINYYYYGMDVWGQGTTVTVSS

SEQ ID NO: 39 - protein, 24 aa, synthetic construct
  REGION 1..24: note = 20B5
QEVINYYYYGMDVWGQGTTVTVSS

SEQ ID NO: 40 - protein, 26 aa, synthetic construct
  REGION 1..26: note = 19F4
LEMATINYYYYGMDVWGQGTMVTVSS

SEQ ID NO: 41 - protein, 24 aa, synthetic construct
  REGION 1..24: note = 19E1
QEFGNYYYYGMDVWGQGTTVTVSS

SEQ ID NO: 42 - protein, 24 aa, synthetic construct
  REGION 1..24: note = 19G8
QEDGNPYYFGMDFWGQGTTVTVSS

SEQ ID NO: 43 - protein, 22 aa, synthetic construct
  REGION 1..22: note = 20H10
GSSYYYDGMDVWGQGTTVTVSS

SEQ ID NO: 44 - protein, 25 aa, synthetic construct
  REGION 1..25: note = 18D10
LENDYGYYYYGMDVWGQGTTVTVSS

SEQ ID NO: 45 - protein, 23 aa, synthetic construct
  REGION 1..23: note = 16F2
RGGLSPLYGMDVWGQGTTVTVSS

SEQ ID NO: 46 - DNA, 15 bp, synthetic construct
  misc_feature 1..15: note = Rat Switch
  source 1..15: mol_type = other DNA
gggctgggctgggct

SEQ ID NO: 47 - DNA, 20 bp, synthetic construct
  misc_feature 1..20: note = Rat Switch
  source 1..20: mol_type = other DNA
gggctgggctgggctgggct

SEQ ID NO: 48 - DNA, 25 bp, synthetic construct
  misc_feature 1..25: note = Rat Switch
  source 1..25: mol_type = other DNA
gggctgggctgggctgggctgggct

SEQ ID NO: 49 - DNA, 30 bp, synthetic construct
  misc_feature 1..30: note = Rat Switch
  source 1..30: mol_type = other DNA
gggctgggctgggctgggctgggctgggct

SEQ ID NO: 50 - DNA, 10 bp, synthetic construct
  misc_feature 1..10: note = Rat Switch
  repeat_region 1..5: note = Repeat 6 to 81 times
  source 1..10: mol_type = other DNA
gggctgggct

SEQ ID NO: 51 - protein, 6 aa, synthetic construct
  REGION 1..6: note = IGHV4-4*02 CDR1
SSNWWS

SEQ ID NO: 52 - protein, 6 aa, synthetic construct
  REGION 1..6: note = 12D10 CDR1
SGNWWS

SEQ ID NO: 53 - protein, 6 aa, synthetic construct
  REGION 1..6: note = 1283 CDR1
RSNWWS

SEQ ID NO: 54 - protein, 7 aa, synthetic construct
  REGION 1..7: note = IGHV6-1*01 CDR1
SNSAAWN

SEQ ID NO: 55 - protein, 7 aa, synthetic construct
  REGION 1..7: note = 4A12 CDR1
SNSAAWN

SEQ ID NO: 56 - protein, 16 aa, synthetic construct
  REGION 1..16: note = IGHV4-4*02 CDR2
EIYHSGSTNYNPSLKS

SEQ ID NO: 57 - protein, 16 aa, synthetic construct
  REGION 1..16: note = 12D10 CDR2
EIYHSGNTNYNPSLKS

SEQ ID NO: 58 - protein, 16 aa, synthetic construct
  REGION 1..16: note = 1283 CDR2
EIYHSGSTNYNPSLKS

SEQ ID NO: 59 - protein, 18 aa, synthetic construct
  REGION 1..18: note = IGHV6-1*01 CDR2
RTYYRSKWYNDYAVSVKS

SEQ ID NO: 60 - protein, 18 aa, synthetic construct
  REGION 1..18: note = 4A12 CDR2
RTYYRSKWYNDYKVSVKS

SEQ ID NO: 61 - protein, 12 aa, synthetic construct
  REGION 1..12: note = 12D10 CDR3
GPLTGEKYYFDL

SEQ ID NO: 62 - protein, 8 aa, synthetic construct
  REGION 1..8: note = 1283 CDR3
IGDWYFDL

SEQ ID NO: 63 - protein, 16 aa, synthetic construct
  REGION 1..16: note = 4A12 CDR3
EGSHSGSGWYLDAFDI

SEQ ID NO: 64 - protein, 17 aa, synthetic construct
  REGION 1..17: note = IGHJ2*01 J-region
YWYFDLWGRGTLVTVSS

SEQ ID NO: 65 - protein, 16 aa, synthetic construct
  REGION 1..16: note = 12D10 J-region
YYFDLWGRGTLVTVSS

SEQ ID NO: 66 - protein, 16 aa, synthetic construct
  REGION 1..16: note = 1283 J-Region
WYFDLWGRGTLVTVSS

SEQ ID NO: 67 - protein, 16 aa, synthetic construct
  REGION 1..16: note = IGHJ3*01 J-Region
DAFDVWGQGTMVTVSS

SEQ ID NO: 68 - protein, 16 aa, synthetic construct
  REGION 1..16: note = 4A12 J-region
DAFDIWGQGTKVTVSS

(19) United States
(12) Patent Application Publication
(10) Pub. No.: US 2024/0057572 A1
(43) Pub. Date: Feb. 22, 2024

(54) Animal Models and Therapeutic Molecules

(71) Applicant: Kymab Limited, Cambridge (GB)

(72) Inventors: Allan Bradley, Cambridge (GB); E-Chiang Lee, Cambridge (GB); Qi Liang, Cambridge (GB); Wei Wang, Cambridge (GB)

(21) Appl. No.: 17/943,533

(22) Filed: Sep. 13, 2022

Related U.S. Application Data
(62) Division of application No. 16/870,413, filed on May 8, 2020.
Application No. 16/870,413 is a continuation of application No. 13/310,431, filed on Dec. 2, 2011.
Application No. 13/310,431 is a continuation-in-part of application No. PCT/GB2010/051122, filed on Jul. 7, 2010.
Application No. 13/310,431 is a continuation-in-part of application No. PCT/GB2011/050019, filed on Jan. 7, 2011.
(60) Provisional application No. 61/223,960, filed on Jul. 8, 2009.
(60) Provisional application No. 61/355,666, filed on Jun. 17, 2010.

(30) Foreign Application Priority Data
Jul. 8, 2009 (GB) 0911846.4
Jul. 28, 2009 (GB) 0913102.0

(51) Int. Cl. A01K 67/027; A61K 39/02; A61K 39/35; C07K 16/00; C07K 16/12; C07K 16/18; C07K 16/46; C12N 15/85

(52) U.S. Cl. CPC A01K 67/0278; A01K 67/0271; A01K 67/0275; A01K 67/0276; A61K 39/107; A61K 39/35; C07K 16/00; C07K 16/1203; C07K 16/1239; C07K 16/18; C07K 16/462; C12N 15/8509; A01K 2207/15; A01K 2217/072; A01K 2217/075; A01K 2217/15; A01K 2227/105; A01K 2267/01; A61K 2039/505; C07K 2317/14; C07K 2317/21; C07K 2317/24; C07K 2317/51; C07K 2317/515; C07K 2317/52; C07K 2317/56; C07K 2317/565; C07K 2317/567; C07K 2317/76; C07K 2317/92

(57) ABSTRACT

The invention discloses methods for the generation of chimaeric human—non-human antibodies and chimaeric antibody chains, antibodies and antibody chains so produced, and derivatives thereof including fully humanised antibodies; compositions comprising said antibodies, antibody chains and derivatives, as well as cells, non-human mammals and vectors, suitable for use in said methods.

### CROSS-REFERENCE TO RELATED APPLICATIONS

This application is a divisional application of U.S. application Ser. No. 16/870,413, which is a continuation of U.S. application Ser. No. 13/310,431, filed Dec. 2, 2011, which is a continuation-in-part application of PCT/GB2010/051122, filed Jul. 7, 2010, which claims the benefit of U.S. Provisional Application No. 61/223,960, filed Jul. 8, 2009; U.S. Provisional Application No. 61/355,666 filed Jun. 17, 2010; GB Patent Application No. 0911846.4, filed Jul. 8, 2009; and GB Patent Application No. 0913102.0, filed Jul. 28, 2009, the entire contents of each of which are incorporated herein by reference in their entirety. U.S. application Ser. No. 13/310,431, filed Dec. 2, 2011, is also a continuation-in-part application of PCT/GB2011/050019, filed Jan. 7, 2011, the entire contents of which is incorporated herein by reference in its entirety.

### SEQUENCE LISTING

The attached sequence listing, titled “39080-18901-20230921.xml (of size 138 kb), created on Sep. 21, 2023, is hereby incorporated by reference in its entirety.

### BACKGROUND

The present invention relates inter glia to non-human animals and cells that are engineered to contain exogenous DNA, such as human immunoglobulin gene DNA, their use in medicine and the study of disease, methods for production of non-human animals and cells, and antibodies and antibody chains produced by such animals and derivatives thereof.

In order to get around the problems of humanizing antibodies a number of companies set out to generate mice with human immune systems. The strategy used was to knockout the heavy and light chain loci in ES cells and complement these genetic lesions with transgenes designed to express the human heavy and light chain genes. Although fully human antibodies could be generated, these models have several major limitations:
(i) The size of the heavy and light chain loci (each several Mb) made it impossible to introduce the entire loci into these models. As a result the transgenic lines recovered had a very limited repertoire of V-regions, most of the constant regions were missing and important distant enhancer regions were not included in the transgenes.
(ii) The very low efficiency of generating the large insert transgenic lines and the complexity and time required to cross each of these into the heavy and light chain knockout strains and make them homozygous again, restricted the number of transgenic lines which could be analysed for optimal expression.
(iii) Individual antibody affinities rarely reached those which could be obtained from intact (non-transgenic) animals.

WO2007117410 discloses chimaeric constructs for expressing chimaeric antibodies.

WO2010039900 discloses knock in cells and mammals having a genome encoding chimaeric antibodies.

The present invention provides, inter alia, a process for the generation in non-human mammals of antibodies that comprise a human Ig variable region, and further provides non-human animal models for the generation of such antibodies.

### SUMMARY OF THE INVENTION

All nucleotide co-ordinates for the mouse are those corresponding to NCBI m37 for the mouse C57BL/6J strain, e.g. April 2007 ENSEMBL Release 55.37h, e.g. NCBI37 Jul. 2007 (NCBI build 37) (e.g. UCSC version mm9 see World Wide Web (www) genome.ucsc.edu and World Wide Web (www) genome.ucsc.edu/FAQ/FAQreleases.html) unless otherwise specified. Human nucleotides coordinates are those corresponding to GRCh37 (e.g. UCSC version hg 19, World Wide Web (www) genome.ucsc.edulFAQ/FAQreleases.html), Feb 2009 ENSEMBL Release 55.37, or are those corresponding to NCBl36, Ensemble release 54 unless otherwise specified. Rat nucleotides are those corresponding to RGSC 3.4 Dec. 2004 ENSEMBL release 55.34w, or Baylor College of Medicine HGSC v3.4 Nov. 2004 (e.g., UCSC m4, see World Wide Web (www) genome.ucsc.edu and World Wide Web (www) genome.ucsc.edu/FAQ/FAQreleases.html) unless otherwise specified.

In the present invention, methods are disclosed for constructing a chimaeric human heavy and light chain loci in a non-human mammal, for example a mouse. Reference to work in mice herein is by way of example only, and reference to mice is taken to include reference to all non-human mammals unless otherwise apparent from the disclosure, with mice being preferred as the non-human mammal.

In one aspect the invention relates to a non-human mammal whose genome comprises:
(a) a plurality of human IgH V regions, one or more human D regions and one or more human J regions upstream of the host non-human mammal constant region; and
(b) optionally one or more human Ig light chain kappa V regions and one or more human Ig light chain kappa J regions upstream of the host non-human mammal kappa constant region and/or one or more human Ig light chain lambda V regions and one or more human Ig light chain lambda J regions upstream of the host non-human mammal lambda constant region;
wherein the non-human mammal is able to produce a repertoire of chimaeric antibodies, or chimaeric light or heavy chains, having a non-human mammal constant region and a human variable region.

In one aspect the invention relates to non-human mammal whose genome comprises
(a) a plurality of human Ig light chain kappa V regions and one or more human Ig light chain kappa J regions upstream of the host non-human mammal kappa constant region and/or a plurality of human Ig light chain lambda V regions and one or more human Ig light chain lambda J regions upstream of the host non-human mammal lambda constant region; and
(b) optionally one or more human IgH V regions, one or more human D regions and one or more human J regions upstream of the host non-human mammal constant region;

wherein the non-human mammal is able to produce a repertoire of chimaeric antibodies, or chimaeric light or heavy chains, having a non-human mammal constant region and a human variable region.

In one aspect the invention relates to non-human mammalian cell whose genome comprises
(a) a plurality of human IgH V regions, one or more human D regions and one or more human J regions upstream of the host non-human mammal constant region and
(b) optionally one or more human Ig light chain kappa V regions and one or more human Ig light chain kappa J regions upstream of the host non-human mammal kappa constant region and/or one or more human Ig light chain lambda V regions and one or more human Ig light chain lambda J regions upstream of the host non-human mammal lambda constant region.

In one aspect the invention relates to a non-human mammalian cell whose genome comprises
(a) a plurality of human Ig light chain kappa V regions and one or more human Ig light chain kappa J regions upstream of the host non-human mammal kappa constant region and/or a plurality of human Ig light chain lambda V regions and one or more human Ig light chain lambda J regions upstream of the host non-human mammal lambda constant region; and
(b) optionally one or more human IgH V regions, one or more human D regions and one or more human J regions upstream of the host non-human mammal constant region;

In a further aspect the invention relates to a method for producing a non-human cell or mammal comprising inserting into a non-human mammal cell genome, such as an ES cell genome;
(a) a plurality of human IgH V regions, one or more human D regions and one or more human J regions upstream of the host non-human mammal constant region; and
(b) optionally one or more human Ig light chain kappa V regions and one or more human Ig light chain kappa J regions upstream of the host non-human mammal kappa constant region and/or one or more human Ig light chain lambda V regions and one or more human Ig light chain lambda J regions upstream of the host non-human mammal lambda constant region; respectively, the insertion being such that the non-human cell or mammal is able to produce a repertoire of chimaeric antibodies having a non-human mammal constant region and a human variable region, wherein steps (a) and (b) can be carried out in either order and each of steps (a) and (b) can be carried out in a stepwise manner or as a single step. Insertion may be by homologous recombination.

In a further aspect the invention relates to a method for producing an antibody or antibody chain specific to a desired antigen the method comprising immunizing a transgenic non-human mammal as disclosed herein with the desired antigen and recovering the antibody or antibody chain.

In a further aspect the invention relates to a method for producing a fully humanised antibody comprising immunizing a transgenic non-human mammal as disclosed herein with the desired antigen, recovering the antibody or cells producing the antibody and then replacing the non-human mammal constant region with a human constant region, for example by protein or DNA engineering.

In a further aspect the invention relates to humanised antibodies and antibody chains produced according to the present invention, both in chimaeric (for example, mouse-human) and fully humanised form, as well as fragments and derivatives of said antibodies and chains, and use of said antibodies, chains and fragments in medicine, including diagnosis.

In a further aspect the invention relates to use of a non-human mammal as described herein as a model for the testing of drugs and vaccines.

In one aspect the invention relates to a non-human mammal whose genome comprises:
(a) a plurality of human IgH V regions, one or more human D regions and one or more human J regions upstream of the host non-human mammal constant region; and
(b) optionally one or more human Ig light chain kappa V regions and one or more human Ig light chain kappa J regions upstream of the host non-human mammal kappa constant region and/or one or more human Ig light chain lambda V regions and one or more human Ig light chain lambda J regions upstream of the host non-human mammal lambda constant region;

wherein the non-human mammal is able to produce a repertoire of chimaeric antibodies or antibody chains having a non-human mammal constant region and a human variable region.

In a further aspect the invention relates to a non-human mammal whose genome comprises:
(a) a plurality of human Ig light chain kappa V regions and one or more human Ig light chain kappa J regions upstream of the host non-human mammal kappa constant region and/or a plurality of human Ig light chain lambda V regions and one or more human Ig light chain lambda J regions upstream of the host non-human mammal lambda constant region: and
(b) optionally one or more human IgH V regions, one or more human D regions and one or more human J regions upstream of the host non-human mammal constant:

wherein the non-human mammal is able to produce a repertoire of chimaeric antibodies having a non-human mammal constant region and a human variable region.

Optionally the non-human mammal genome is modified to prevent expression of fully host-species specific antibodies.

In one aspect the inserted human DNA comprises at least 50% of the human heavy chain variable (V) genes, such as at least 60%, at least 70%, at least 80%, at least 90%, and in one aspect all of the human V genes.

In one aspect the inserted human DNA comprises at least 50% of the human heavy chain diversity (D) genes, such as at least 60%, at least 70%, at least 80%, at least 90%, and in one aspect all of the human D genes.

In one aspect the inserted human DNA comprises at least 50% of the human heavy chain joining (J) genes, such as at least 60%, at least 70%, at least 80%, at least 90%, and in one aspect all of the human J genes.

In one aspect the inserted human DNA comprises at least 50% of the human light chain Variable (V) genes, such as at least 60%, at least 70%, at least 80%, at least 90%, and in one aspect all of the human light chain V genes.

In one aspect the inserted human DNA comprises at least 50% of the human light chain joining (J) genes, such as at least 60%, at least 70%, at least 80%, at least 90%, and in one aspect all of the human light chain J genes.

The inserted human genes may be derived from the same individual or different individuals, or be synthetic or represent human consensus sequences.

Although the number of V D and J regions is variable between human individuals, in one aspect there are considered to be 51 human V genes, 27 D and 6 J genes on the heavy chain, 40 human V genes and 5 J genes on the kappa light chain and 29 human V genes and 4 J genes on the lambda light chain (Janeway and Travers, Immunobiology, Third edition)

In one aspect the human heavy chain locus inserted into the non-human mammal contains the full repertoire of human V, D and J regions, which in the genome is in functional arrangement with the non-human mammal constant regions such that functional chimaeric antibodies can be produced between the human variable and non-human mammal constant regions. This total inserted human heavy chain genetic material is referred to herein as the human IgH VDJ region, and comprises DNA from a human genome that encodes all the exons encoding human V,D and J portions and suitably also the associated introns. Similarly, reference to the human Ig light chain kappa V and J regions herein refers to human DNA comprising all the exons encoding V and J regions and suitably also the associated introns of the human genome. Reference to the human Ig light chain lambda V and J regions herein refers to human DNA comprising all the exons encoding V and J regions and suitably also the associated introns of the human genome.

Human variable regions are suitably inserted upstream of a non-human mammal constant region, the latter comprising all of the DNA required to encode the full constant region or a sufficient portion of the constant region to allow the formation of an effective chimaeric antibody capable of specifically recognising an antigen.

In one aspect the chimaeric antibodies or antibody chains have a part of a host constant region sufficient to provide one or more effector functions seen in antibodies occurring naturally in a host mammal, for example that they are able interact with Fc receptors, and/or bind to complement.

Reference to a chimaeric antibody or antibody chain having a host non mammal constant region herein therefore is not limited to the complete constant region but also includes chimaeric antibodies or chains which have all of the host constant region, or a part thereof sufficient to provide one or more effector functions. This also applies to non-human mammals and cells and methods of the invention in which human variable region DNA may be inserted into the host genome such that it forms a chimaeric antibody chain with all or part of a host constant region. In one aspect the whole of a host constant region is operably linked to human variable region DNA.

The host non-human mammal constant region herein is preferably the endogenous host wild-type constant region located at the wild type locus, as appropriate for the heavy or light chain. For example, the human heavy chain DNA is suitably inserted on mouse chromosome 12, suitably adjacent the mouse heavy chain constant region.

In one aspect the insertion of the human DNA, such as the human VDJ region is targeted to the region between the J4 exon and the CIJ locus in the mouse genome IgH locus, and in one aspect is inserted between co-ordinates 114,667,090 and 114,665,190, or at co-ordinate 114,667,091, after 114,667,090. In one aspect the insertion of the human DNA, such as the human light chain kappa VJ is targeted into mouse chromosome 6 between co-ordinates 70,673,899 and 70,675,515, suitably at position 70,674,734, or an equivalent position in the lambda mouse locus on chromosome 16.

In one aspect the host non-human mammal constant region for forming the chimaeric antibody may be at a different (non endogenous) chromosomal locus. In this case the inserted human DNA, such as the human variable VDJ or VJ region(s) may then be inserted into the non-human genome at a site which is distinct from that of the naturally occurring heavy or light constant region. The native constant region may be inserted into the genome, or duplicated within the genome, at a different chromosomal locus to the native position, such that it is in a functional arrangement with the human variable region such that chimaeric antibodies of the invention can still be produced.

In one aspect the human DNA is inserted at the endogenous host wild-type constant region located at the wild type locus between the host constant region and the host VDJ region.

Reference to location of the variable region upstream of the non-human mammal constant region means that there is a suitable relative location of the two antibody portions, variable and constant, to allow the variable and constant regions to form a chimaeric antibody or antibody chain in vivo in the mammal. Thus, the inserted human DNA and host constant region are in functional arrangement with one another for antibody or antibody chain production.

In one aspect the inserted human DNA is capable of being expressed with different host constant regions through isotype switching. In one aspect isotype switching does not require or involve trans switching. Insertion of the human variable region DNA on the same chromosome as the relevant host constant region means that there is no need for trans-switching to produce isotype switching.

As explained above, the transgenic loci used for the prior art models were of human origin, thus even in those cases when the transgenes were able to complement the mouse locus so that the mice produced B-cells producing fully human antibodies, individual antibody affinities rarely reached those which could be obtained from intact (non-transgenic) animals. The principal reason for this (in addition to repertoire and expression levels described above) is the fact that the control elements of the locus are human. Thus, the signalling components, for instance to activate hyper-mutation and selection of high affinity antibodies are compromised.

In contrast, in the present invention, host non-human mammal constant regions are maintained and it is preferred that at least one non-human mammal enhancer or other control sequence, such as a switch region, is maintained in functional arrangement with the non-human mammal constant region, such that the effect of the enhancer or other control sequence, as seen in the host mammal, is exerted in whole or in part in the transgenic animal.

This approach above is designed to allow the full diversity of the human locus to be sampled, to allow the same high expression levels that would be achieved by non-human mammal control sequences such as enhancers, and is such that signalling in the B-cell, for example isotype switching using switch recombination sites, would still use non-human mammal sequences.

A mammal having such a genome would produce chimaeric antibodies with human variable and non-human mammal constant regions, but these could be readily humanized, for example in a cloning step. Moreover the in vivo efficacy of these chimaeric antibodies could be assessed in these same animals.

In one aspect the inserted human IgH VDJ region comprises, in germline configuration, all of the V, D and J regions and intervening sequences from a human.

In one aspect 800-1000 kb of the human IgH VDJ region is inserted into the non-human mammal IgH locus, and in one aspect a 940, 950 or 960 kb fragment is inserted. Suitably this includes bases 105,400,051 to 106,368,585 from human chromosome 14.

In one aspect the inserted IgH human fragment consists of bases 105,400,051 to 106.368.585 from chromosome 14. In one aspect the inserted human heavy chain DNA. such as DNA consisting of bases 105,400,051 to 106,368,585 from chromosome 14, is inserted into mouse chromosome 12 between the end of the mouse J4 region and the Ep region, suitably between co-ordinates 114,667,090 and 114,665,190, or at co-ordinate 114,667,091, after 114,667,090 In one aspect the insertion is between co-ordinates 114,667,089 and 114,667,090 (co-ordinates refer to NCBI m37, for the mouse C57BL/6J strain), or at equivalent position in another non-human mammal genome.

In one aspect the inserted human kappa VJ region comprises, in germline configuration, all of the V and J regions and intervening sequences from a human. Suitably this includes bases 88,940,356 to 89,857,000 from human chromosome 2, suitably approximately 917 kb. In a further aspect the light chain VJ insert may comprise only the proximal clusters of V segments and J segments. Such an insert would be of approximately 473 kb. In one aspect the human light chain kappa DNA, such as the human IgK fragment of bases 88,940,356 to 89,857,000 from human chromosome 2, is suitably inserted into mouse chromosome 6 between co-ordinates 70,673,899 and 70,675,515, suitably at position 70,674,734. These co-ordinates refer to NCBl36 for the human genome, ENSEMBL Release 54 and NCBIM37 for the mouse genome, relating to mouse strain C57BL/6J.

In one aspect the human lambda VJ region comprises, in germline configuration, all of the V and J regions and intervening sequences from a human.

Suitably this includes analogous bases to those selected for the kappa fragment, from human chromosome 2.

A cell or non-human mammal of the invention, in one embodiment, comprises an insertion of human heavy chain variable region DNA between co-ordinates 114, 666, 183 and 114, 666, 725, such as between 114 666 283 and 114 666 625, optionally between co-ordinates 114,666,335 and 114,666,536, optionally between 114,666,385 and 114,666,486, or between 114,666,425 and 114,666,446, or between 114,666,435 and 114,666,436 of mouse chromosome 12 with reference to NCBIM37 for the mouse genome, relating to mouse strain C57BLi6J or an equivalent position of mouse chromosome 12 from a different mouse strain or an equivalent position in the genome of another non-human vertebrate, e.g., a rat. The insertion between co-ordinates 114,666,435 and 114,666,436 relating to mouse strain C57BLi6J is equivalent to an insertion between co-ordinates 1207826 and 1207827 on chromosome 12 with reference to the 129/SvJ genomic sequence of the GenBank® access number NT114985.2. An insertion may be made at equivalent position in another genome, such as another mouse genome. In an example of this embodiment, the cell or mammal of the invention comprises a human IgH VDJ region which comprises or consists of nucleotides 106,328,851-107,268,544, such as nucleotides 106,328,901-107,268,494, such as nucleotides 106,328,941-107,268,454, such as nucleotides 106,328,951-107,268,444 of human Chromosome 14, with reference to the GRCH37/hg19 sequence database, or insertion of equivalent nucleotides relating to chromosome 14 from a different human sequence or database. The human insertion may be made between the regions indicated above.

A cell or mammal of the invention, in one embodiment, comprises an insertion of the human kappa VJ region, suitably comprising or consisting of, in germline configuration, all of the V and J regions and intervening sequences from a human, the insertion of the human DNA being made between co-ordinates 70,673,918-70,675,517, such as between co-ordinates 70, 674,418 and 70 675, 017, such as between co-ordinates 70,674, 655-70,674,856, such as between co-ordinates 70,674, 705-70,674,906, such as between co-ordinates 70,674, 745-70,674,766, such as between co-ordinates 70,674,755 and 70,674,756 of mouse chromosome 6, numbering with reference to NCBIM37 for the mouse genome, relating to mouse strain C57BL/6J, or an insertion at an equivalent position in another genome, such as another mouse genome. In an example of this embodiment, a cell or mammal of the invention comprises an insertion of nucleotides 89,159,079-89,630,437 and/or 89,941,714-90,266,976 of human chromosome 2 with reference to the GRCH37/hg19 sequence database (or equivalent nucleotides relating to chromosome 2 from a different human sequence or database), such as an insertion of these 2 discrete fragments without the intervening sequence, or an insertion of the complete 89,159,079-90,266,976 region.

The insertion may comprise, or consist, of:
(i) nucleotides 89,158,979-89,630,537, such as 89,159,029-89,630,487, such as 89,159,069-89,630,447, such as 89,159,079-89,630,437, optionally in addition to fragment (ii) below
(ii) nucleotides 89,941,614-90,267,076, such as 89,941,664-90,267,026, such as 89, 941,704-90,266,986, such as 89,941,714-90,266,976; optionally in addition to fragment (i)
(iii) nucleotides 89,158,979-90,267,076, such as nucleotides 89,159,079-90,266,976.

The human insertion may be made between the regions indicated above.

In an embodiment, a cell or mammal of the invention comprises an insertion of a human lambda region which comprises at least one human Jλ region (eg, a germline region) and at least one human CA region (eg, a germline region), optionally C_{λ}6 and/or C_{λ}7. For example, the cell or mammal comprises a plurality of human Jλ regions, optionally two or more of J_{λ}1, J_{λ}2, J_{λ}6 and J_{λ}7, optionally all of J_{λ}1, J_{λ}2, J_{λ}6 and J_{λ}7. In an example, the cell or mammal comprises at least one human J_{λ}-C_{λ} cluster, optionally at least J_{λ}7-C_{λ}7.

In one aspect the human JC cluster is inserted 3′ of the last endogenous J lambda or is inserted 3′ of the last endogenous J kappa region, suitably immediately 3′ of these sequences, or substantially immediately 3′ of these sequences.

In one aspect the insertion into the mouse lambda locus is made downstream of the endogenous C1 gene segment, for example where there is a 3′ J1C1 cluster, suitably immediately 3′ of the C1 segment, or substantially immediately 3′ of the segment.

In one aspect (e.g. cell or non-human mammal) a human JC cluster is inserted into a kappa locus and any resulting cell or animal is heterozygous at that locus, such that the cell has one chromosome with human lambda DNA inserted into the kappa locus, and another chromosome with human kappa DNA at the endogenous kappa locus.

In an embodiment, a cell or mammal of the invention comprises a human EA enhancer.

A cell or mammal may of the invention comprise an inserted human lambda VJ region, suitably comprising or consisting of, in germline configuration, all of the V and J regions and intervening sequences from a human, the inserted region comprises or consisting of nucleotides 22,375,509-23,327,984, such as nucleotides 22,375,559-23,327,934, such as nucleotides 22,375,599-23,327,894, such as nucleotides 22,375,609-23,327,884 from human Chromosome 22, with reference to the GRCH371hg19 sequence database, or equivalent DNA from another human sequence or database. The insertion into the mouse genome may be made between co-ordinates 19,027,763 and 19,061,845, such as between co-ordinates 19, 037, 763 and 19, 051, 845, such as between co-ordinates 19,047,451 and 19,047,652, such as between co-ordinates 19,047,491 and 19,047,602, such as between co-ordinates 19,047,541 and 19,047,562, such as between co-ordinates 19,047,551 and 19,047,552 of mouse Chromosome 16 (with reference to NCBIM37 for the mouse genome, relating to mouse strain C57B116J, equivalent to co-ordinates 1,293,646-1,293,647 of the 129 SvJ genomic sequence in the sequence file of NT_039630.4), or may be an insertion at an equivalent position in other genome, such as another mouse genome. The insertion of the human lambda nucleic acid into the mouse genome may alternatively be made between co-ordinates 70,673,918 and 70,675,517, such as between co-ordinates 70, 674,418 and 70 675, 017, such as between co-ordinates 70,674,655 and 70,674,856, such as between co-ordinates 70,674,705 and 70,674,806, such as between co-ordinates 70,674,745 and 70,674,766, such as between co-ordinates 70,674,755 and 70,674,756 of mouse Chromosome 6 (with reference to NCBIM37 for the mouse genome, relating to mouse strain C57BL/6J) or equivalent in another genome. The human insertion may be made between the regions indicated above.

All specific human fragments described above may vary in length, and may for example be longer or shorter than defined as above, such as 500 bases, 1 KB, 2K, 3K, 4K, 5 KB, 10 KB, 20 KB, 30 KB, 40 KB or 50 KB or more, which suitably comprise all or part of the human V(D)J region, whilst preferably retaining the requirement for the final insert to comprise human genetic material encoding the complete heavy chain region and light chain region, as appropriate, as described above.

In one aspect the 5′ end of the human insert described above is increased in length. Where the insert is generated in a stepwise fashion then the increase in length is generally in respect of the upstream (5′) clone.

In one aspect the 3′ end of the last inserted human gene, generally the last human J gene to be inserted is less than 2 kb, preferably less than 1 KB from the human-mouse join region.

In one aspect the non-human mammal comprises some or all of the human light chain kappa VJ region as disclosed herein but not the human light chain lambda VJ region.

In one aspect the cell or non-human mammal comprises a fully human lambda locus (lambda VJC regions from a human), a chimaeric kappa locus (human kappa VJ regions operatively linked to a host kappa constant region) and a chimaeric heavy chain locus, having a human VDJ region operatively linked to a host heavy chain constant region.

In a further aspect the genome comprises an insertion of V, D (heavy chain only) and J genes as described herein at the heavy chain locus and one light chain locus, or at the heavy chain locus and both light chain loci. Preferably the genome is homozygous at one, or both, or all three loci.

In another aspect the genome may be heterozygous at one or more of the loci, such as heterozygous for DNA encoding a chimaeric antibody chain and native (host cell) antibody chain. In one aspect the genome may be heterozygous for DNA capable of encoding 2 different antibody chains of the invention, for example, comprising 2 different chimaeric heavy chains or 2 different chimaeric light chains.

In one aspect the invention relates to a non-human mammal or cell, and methods for producing said mammal or cell, as described herein, wherein the inserted human DNA, such as the human IgH VDJ region and/or light chain V, J regions are found on only one allele and not both alleles in the mammal or cell. In this aspect a mammal or cell has the potential to express both an endogenous host antibody heavy or light chain and a chimaeric heavy or light chain.

In a further aspect of the invention the human VDJ region, or light chain VJ region, is not used in its entirety, but parts of the equivalent human VDJ or VJ region, such as the exons, from other species may be used, such as one or more V, D, or J exons from other species, or regulatory sequences from other species. In one aspect the sequences used in place of the human sequences are not human or mouse. In one aspect the sequences used may be from rodent, or, primate such as chimp. For example, 1, 2, 3, 4, or more, or all of the J regions from a primate other than a human may be used to replace, one, 2, 3, 4, or more or all of the human J exons in the VDJNJ region of the cells and animals of the invention.

In a further aspect the inserted human DNA, such as the human IgH VDJ region, and/or light chain VJ regions, may be inserted such that they are operably linked in the genome with a mu constant region from a non-human, non-mouse species, such as a rodent or primate sequence, such as a rat sequence.

Other non-human, non-mouse species from which DNA elements may be used in the present invention include rabbits, lamas, dromedary, alpacas, camels and sharks.

In one aspect the inserted human DNA, such as the human VDJ or VJ region, is not operably linked to the endogenous host mu sequence but rather to a non-host mu sequence.

Operable linkage suitably allows production of an antibody heavy or light chain comprising the human variable region.

In one aspect the inserted human DNA, such as the human IgH VDJ region (and/or light chain VJ regions) may be inserted into the host chromosome together with mu constant region nucleic acid which is not host mu constant region nucleic acid, and preferably is a mu constant region from a non-mouse, non-human species. Suitably the inserted human DNA, such as the human VDJ region (and/or light chain VJ regions) is operably linked to a non-human, non-mouse mu, and is able to form a chimaeric antibody heavy or light chain. In another aspect a non-mouse, non-human mu may be inserted into the host chromosome on a separate genetic element to that of the human variable region, or at a different location in the genome, suitably operably linked to the variable region such that a chimaeric antibody heavy or light can be formed.

In an additional aspect the invention relates to a non-human mammal or a cell whose genome comprises a plurality of human IgH V regions, one or more human D regions and one or more human J regions upstream of a host non-human mammal light chain constant region, arranged such that the cell or mammal is able to express a chimaeric antibody chain. The invention also relates to a non-human mammal or a cell whose genome additionally or alternatively comprises a plurality of human Ig light chain V regions, and one or more human J regions upstream of a host non-human mammal heavy chain constant region, such that the cell or mammal is able to express a chimaeric antibody chain. The cell or mammal may be able to express an antibody having both heavy and light chains, including at least one chimaeric antibody chain, as disclosed above.

The inserted human heavy chain variable regions may be any of those described herein, and may be inserted at the positions described above for insertion 5′ of the lambda and kappa constant regions. Likewise the inserted human light chain variable regions may be those described above, and may be inserted at the positions described above for insertion 5′ of the heavy chain constant region.

For example, the genome or the cell or non-human mammal of the invention may encode an antibody comprising an antibody chain having a human heavy chain variable region upstream of a mouse light chain constant region; or an antibody chain having a human light chain variable region upstream of a mouse heavy chain constant region, in combination with one of:
a fully human antibody light chain;
a fully human antibody heavy chain;
a non-human vertebrate (e.g., mouse or rat) antibody light chain;
a non-human vertebrate (e.g., mouse or rat) antibody heavy chain;
a chimaeric non-human vertebrate (e.g., mouse or rat)—human antibody chain;
an antibody chain having a human heavy chain variable region upstream of a non-human vertebrate (e.g., mouse or rat) light chain constant region;
an antibody chain having a human light chain variable region upstream of a non-human vertebrate (e.g., mouse or rat) heavy chain constant region.

The invention also relates to a transgene encoding a plurality of human IgH V regions, one or more human D regions and one or more human J regions upstream of a host non-human mammal light chain constant region, optionally comprised within a vector.

The invention also relates to a transgene encoding a plurality of human Ig light chain V regions, and one or more human light chain J regions upstream of a host non-human mammal heavy chain constant region, optionally comprised within a vector.

In one aspect the invention relates to a cell, or non-human mammal, the genome of which comprises: one or more human Ig light chain kappa V regions and one or more human Ig light chain kappa J regions upstream of all or part of the human kappa constant region.

In another aspect the invention relates to a cell, or non-human mammal, the genome of which comprises: one or more human Ig light chain lambda V regions and one or more human Ig light chain lambda J regions upstream of all or part of the human lambda constant region.

Suitably the light chain VJ and C regions are able to form antibody chains in vivo capable of specifically reacting with an antigen.

In one aspect of the invention there is no non-human coding sequence in the inserted light chain region.

In such aspects a human kappa and/or lambda region is inserted into the genome, in combination with insertion of the heavy chain VDJ region or part thereof, upstream of the host heavy chain constant region as disclosed herein.

The cell or non-human mammal of the invention may comprise:
(a) a plurality of human IgH V regions, one or more human D regions and one or more human J regions upstream of the host non-human mammal constant region; and
(b) one or more human Ig light chain kappa V regions and one or more human Ig light chain kappa J regions upstream of all or part of the non-human kappa constant region, wherein the non-human mammal is able to produce a repertoire of antibodies having an antibody chain comprising non-human mammal constant region and a human variable region.

The cell or non-human mammal of the invention may comprise
(a) a plurality of human IgH V regions, one or more human D regions and one or more human J regions upstream of the host non-human mammal constant region; and one or more human Ig light chain lambda V regions and one or more human Ig light chain lambda J regions upstream of the host non-human mammal lambda constant region;

wherein the non-human mammal is able to produce a repertoire of antibodies having an antibody chain comprising a non-human mammal constant region and a human variable region.

Suitably the insertion of the human VJC light chain DNA, or part thereof as disclosed above, is made at the equivalent mouse locus. In one aspect the human light chain kappa VJC DNA, or part thereof, is inserted immediately upstream or downstream of the mouse kappa VJC region. In one aspect, the human light chain lambda VJC region or part thereof is inserted immediately upstream or downstream of the mouse lambda VJC region. In one aspect only the human kappa VJC locus is inserted and not the human lambda VJC locus. In one aspect only the human lambda VJC locus is inserted and not the human kappa VJC locus. Insertions may be made using the techniques disclosed herein, and suitably do not remove the host sequences from the genome. In one aspect the non-human mammal host VJC sequences may be inactivated in some way, by mutation, or inversion, or by insertion of the human variable region DNA, or by any other means. In one aspect the cell or non-human mammal of the invention may comprise an insertion of the complete VJC human region.

The human kappa variable region DNA might be inserted into the genome in functional arrangement with a lambda constant region, for example inserted upstream of a lambda constant region. Alternatively human lambda region variable DNA might be inserted in functional arrangement with a kappa constant region, for example inserted upstream of a kappa constant region.

In one aspect one or more non-human mammal control sequences such as the enhancer sequence(s) is maintained upstream of the nonhuman mammal Mu constant region, suitably in its native position with respect to the distance from the constant region.

In one aspect one or more non-human mammal control sequences such as an enhancer sequence(s) are maintained downstream of the nonhuman mammal Mu constant region, suitably in its native position with respect to the distance from the constant region.

In one aspect a non-human mammal switch sequence, suitably the endogenous switch sequence, is maintained upstream of the non-human mammal Mu constant region, suitably in its native position with respect to distance from the constant region.

In such location the host enhancer or switch sequences are operative in vivo with the host constant region sequence(s).

In one aspect a switch sequence is neither human, nor native in the non-human mammal, for example in one aspect a non-human mammal switch sequence is not a mouse or human switch sequence. The switch sequence may be, for example, a rodent or primate sequence, or a synthetic sequence. In particular the switch sequence may be a rat sequence where the non-human mammal is a mouse. By way of example, a mouse or human constant mu sequence may be placed under the control of a switch sequence from a rat, or chimp, or other switch sequence, suitably capable of allowing isotype switching to occur in vivo.

In one aspect the switch sequence of the invention is a switch sequence comprising 3, 4, 5, 6 or more (up to 82) contiguous repeats of the repeat sequence GGGCT (SEQ ID no 46-50), such as a rat switch sequence. By “rat switch” herein it is meant that the switch is a wild-type switch corresponding to a switch from a rat genome or derived from such a switch.

In one aspect the switch sequence of the invention is a rat switch sequence comprising the following repeats: GAGCT (296 repeats; SEQ ID No 18), GGGGT (50 repeats; SEQ ID No 19), and GGGCT (83 repeats; SEQ ID No 20).

In one example the rat switch sequence comprises or consists of the sequence of SEQ ID no 1.

In these embodiments, and where the non-human mammal is a mouse or the cell is a mouse cell, the switch is optionally a rat switch as described herein.

Alternatively, the switch sequence present in cells or mammal of the invention is a mouse switch, eg, is from a mouse such as a mouse 129 strain or mouse C57 strain, or from a strain derived therefrom, optionally comprising or consisting of the sequence of SEQ ID no 4 or 5. By “mouse switch” herein it is meant that the switch is a wild-type switch corresponding to a switch from a mouse genome or derived from such a switch. In this embodiment, and where the non-human mammal is a mouse or the cell is a mouse cell, the mouse switch sequence is optionally the endogenous switch or is a mouse switch from another mouse strain.

The cell or mammal of the invention may therefore comprise a human or non-human mammal switch sequence and a human or non-human mammal enhancer region or regions. They may be upstream of a human or non-human mammal constant region. Preferably the control sequences are able to direct expression or otherwise control the production of antibodies comprising a constant region with which they are associated. One combination envisaged is a rat switch with mouse enhancer sequences and mouse constant regions in a mouse cell.

In one aspect the invention relates to a cell, preferably a non-human cell, or non-human mammal comprising an immunoglobulin heavy chain or light chain locus having DNA from 3 or more species. For example, the cell or animal may comprise host cell constant region DNA, one or more human V, D or J coding sequences and one or more non-human, non-host DNA regions that are able to control a region of the immunoglobulin locus, such as a switch sequence, promoter or enhancer which are able to control expression or isotype switching in vivo of the Ig DNA. In one aspect the cell or animal is a mouse and comprises additionally human DNA from the human Ig locus and additionally a non-mouse DNA sequence, such as a rat DNA sequence, capable of regulation of the mouse or human DNA..

In another aspect the invention relates to a cell, preferably non-human cell, or non-human mammal comprising an immunoglobulin heavy chain or light chain locus having DNA from 2 or more different human genomes. For example, it could comprise heavy chain V(D)J sequences from more than one human genome within a heavy or light chain, or heavy chain VDJ DNA from one genome and light chain VJ sequences from a different genome.

In one aspect the invention relates to a DNA fragment or cell or non-human mammal comprising an immunoglobulin heavy chain or light chain locus, or part thereof, having DNA from 2 or more species, where one species contributes a non-coding region such as a regulatory region, and the other species coding regions such as V, D, J or constant regions.

In one aspect the human promoter and/or other control elements that are associated with the different human V, D or J regions are maintained after insertion of the human VDJ into the mouse genome.

In a further aspect one or more of the promoter elements, or other control elements, of the human regions, such as the human V regions, are optimised to interact with the transcriptional machinery of a non-human mammal.

Suitably a human coding sequence may be placed under the control of an appropriate non-human mammal promoter, which allows the human DNA to be transcribed efficiently in the appropriate non-human animal cell. In one aspect the human region is a human V region coding sequence, and a human V region is placed under the control of a non-human mammal promoter.

The functional replacement of human promoter or other control regions by non-human mammal promoter or control regions may be carried out by use of recombineering, or other recombinant DNA technologies, to insert a part of the human Ig region (such as a human V region) into a vector (such as a BAC) containing a non-human Ig region. The recombineering/recombinant technique suitably replaces a portion of the non-human (e.g. mouse) DNA with the human Ig region, and thus places the human Ig region under control of the non-human mammal promoter or other control region. Suitably the human coding region for a human V region replaces a mouse V region coding sequence. Suitably the human coding region for a human D region replaces a mouse D region coding sequence. Suitably the human coding region for a human J region replaces a mouse J region coding sequence. In this way human V, D or J regions may be placed under the control of a non-human mammal promoter, such as a mouse promoter.

In one aspect the only human DNA inserted into the non-human mammalian cell or animal are V, D or J coding regions, and these are placed under control of the host regulatory sequences or other (non-human, non-host) sequences, In one aspect reference to human coding regions includes both human introns and exons, or in another aspect simply exons and no introns, which may be in the form of cDNA.

It is also possible to use recombineering, or other recombinant DNA technologies, to insert a non-human-mammal (e.g. mouse) promoter or other control region, such as a promoter for a V region, into a BAC containing a human Ig region. A recombineering step then places a portion of human DNA under control of the mouse promoter or other control region.

The approaches described herein may also be used to insert some or all of the V, D and J regions from the human heavy chain upstream of a light chain constant region, rather than upstream of the heavy chain constant region. Likewise some or all of the human light chain V and J regions may be inserted upstream of the heavy chain constant region. Insertion may be at the endogenous constant region locus, for example between the endogenous constant and J region, and may be of some, or all, of the V, D or J genes alone, excluding promoter or enhancer sequences, or may be of some, or all, of the V, D or J genes with one or more or all respective promoter or enhancer sequences. In one aspect the full repertoire of V, D or J fragments in germline orientation may be inserted upstream and in functional arrangement with a host constant region.

Thus the present invention allows V and/or D and/or J regions from a human, or any species, to be inserted into a chromosome of a cell from a different species that comprises a constant region, allowing a chimaeric antibody chain to be expressed.

In one aspect the invention requires only that some human variable region DNA is inserted into the genome of a non-human mammal in operable arrangement with some, or all, of the human heavy chain constant region at the region of the endogenous heavy chain constant region locus such that an antibody chain can be produced. In this aspect of the invention and where human light chain DNA is additionally inserted, the light chain DNA insertion can be in the form of a completely human construct, having both human variable DNA and human constant region DNA, or have human variable region DNA and constant region DNA from a non-human, non-host species. Other variations are also possible, such as insertion of both of the light chain human variable region and host genome constant region. In addition the insertion of said light chain transgenes need not be at the equivalent endogenous locus, but may be anywhere in the genome. In such a scenario the cell or mammal may produce chimaeric heavy chains (comprising human variable region DNA and mouse constant region DNA) and light chains comprising human variable and human constant region DNA. Thus in one aspect of the invention the lambda and or kappa human variable region DNA can be inserted upstream of the endogenous locus, or downstream, or indeed on a different chromosome to the endogenous locus, and inserted with or without constant region DNA.

As well insertion of human light chain DNA upstream of the host non-human mammal constant region, a further aspect of the invention relates to insertion of one or both light chain human variable regions downstream of the equivalent endogenous locus constant region, or elsewhere in the genome.

Generally, insertion of human variable region DNA at or close to the equivalent endogenous locus in the recipient genome is preferred, for example within 1, 2, 3, 4, 5, 6, 7, 8, 9, or 10 kb of the boundary (upstream or downstream) of a host immunoglobulin locus.

Thus in one aspect the invention can relate to a cell or non-human mammal whose genome comprises:
(a) a plurality of human IV V regions, one or more human D regions and one or more human J regions upstream of the host non-human mammal constant region; and
(b) one or more human Ig light chain kappa V regions and one or more human Ig light chain kappa J regions, and/or, one or more human Ig light chain lambda V regions and one or more human Ig light chain lambda J regions;

wherein the non-human mammal is able to produce a repertoire of chimaeric antibodies, or chimaeric light or heavy chains, having a non-human mammal constant region and a human variable region.

In one particular aspect the genome of the cell or non-human mammal comprises:
a plurality of human IgH V regions, one or more human D regions and one or more human J regions upstream of the host non-human mammal constant region;
one or more human Ig light chain kappa V regions and one or more human Ig light chain kappa J regions upstream of the host non-human mammal kappa constant region, and
one or more human Ig light chain lambda V regions and one or more human Ig light chain lambda J regions downstream of the host non-human mammal lambda constant region,
optionally in which the human lambda variable region may be inserted upstream or downstream of the endogenous host lambda locus in operable linkage with a human lambda constant region, such that the non-human mammal or cell can produce fully human antibody light chains and chimaeric heavy chains.

In a further, different, aspect of the invention, the use of the methods of the invention allows a locus to be built up in a stepwise manner by sequential insertions, and thus allows for the insertion of human variable DNA together with human or non-human constant region DNA at any suitable location in the genome of a non-human host cell. For example, methods of the invention can be used to insert human immunoglobulin variable region DNA together with constant region DNA from the host genome anywhere in the genome of a non-human host cell, allowing a chimaeric antibody chain to be produced from a site other than the endogenous heavy region. Any human heavy chain or light chain DNA construct contemplated above can be inserted into any desired position into the genome of a non-human host cell using the techniques described herein. The present invention thus also relates to cells and mammals having genomes comprising such insertions.

The invention also relates to a vector, such as a BAC, comprising a human V, D or J region in a functional arrangement with a non-human mammal promoter, or other control sequence, such that the expression of the human V, D or J region is under the control of the non-human mammal promoter in a cell of the non-human mammal, such as an ES cell, in particular once inserted into the genome of that cell.

The invention also relates to cells and non-human mammals containing said cells, which cells or mammals have a human V, D or J region in a functional arrangement with a non-human mammal promoter, or other control sequence, such that the expression of the human V, D or J region is under the control of the non-human mammal promoter in the cells or mammal.

Generally, one aspect of the invention thus relates to a non-human mammal host cell capable of expression of a human V, D or J coding sequence under the control of a host promoter or control region, the expression capable of producing a humanised antibody having a human variable domain and non-human mammal constant region.

In one aspect the invention relates to a cell, such as a non mammalian cell, such as an ES cell, the genome of which comprises
(a) a plurality of human IgH V regions, one or more human D regions and one or more human J regions upstream of the host non-human mammal constant region: and
(b) optionally one or more human Ig light chain kappa V regions and one or more human Ig light chain kappa J regions upstream of the host non-human mammal kappa constant region and/or one or more human Ig light chain lambda V regions and one or more human Ig light chain lambda J regions upstream of the host non-human mammal lambda constant region;

In another aspect the invention relates to a cell, such as a non-human mammal cells, such as ES cells whose genome comprises
(a) a plurality of human Ig light chain kappa V regions and one or more human Ig light chain kappa J regions upstream of the host non-human mammal kappa constant region and/or a plurality of human Ig light chain lambda V regions and one or more human Ig light chain lambda J regions upstream of the host non-human mammal lambda constant region; and
(b) optionally one or more human 10.1 V regions, one or more human D regions and one or more human J regions upstream of the host non-human mammal constant region

In one aspect the cell is an ES cell is capable of developing into a non-human mammal able to produce a repertoire of antibodies which are chimaeric, said chimaeric antibodies having a non-human mammal constant region and a human variable region. Optionally the genome of the cell is modified to prevent expression of fully host-species specific antibodies.

In one aspect the cell is an induced pluripotent stem cell (iPS cell).

In one aspect cells are isolated non-human mammalian cells.

In one aspect a cell as disclosed herein is preferably a non-human mammalian cell.

In one aspect the cell is a cell from a mouse strain selected from C57BL/6, M129 such as 129/SV, BALB/c, and any hybrid of C57BL/6, M129 such as 129/SV, or BALB/c.

The invention also relates to a cell line which is grown from or otherwise derived from cells as described herein, including an immortalised cell line. The cell line may comprise inserted human V, D or J genes as described herein, either in germline configuration or after rearrangement following in vivo maturation. The cell may be immortalised by fusion (eg, electrofusion or using PEG according to standard procedures.) to a tumour cell (eg, P3X63-Ag8.653 (obtainable from LGC Standards; CRL-1580), SP2/0-Ag14 (obtainable from ECACC), NSI or NSO), to provide an antibody producing cell and cell line, or be made by direct cellular immortalisation.

The present invention also relates to vectors for use in the invention. In one aspect such vectors are BACs (bacterial artificial chromosomes). It will be appreciated that other cloning vectors may be used in the invention, and therefore reference to BACs herein may be taken to refer generally to any suitable vector.

In one aspect BACs used for generation of human DNA to be inserted, such as the VDJ or VJ regions are trimmed so that in the final human VDJ or VJ region or part thereof in the non-human mammal, no sequence is duplicated or lost when compared to the original human genomic sequence.

In one aspect the invention relates to a vector comprising an insert, preferably comprising a region of human DNA from some of the human VDJ or VJ locus, flanked by DNA which is not from that locus. The flanking DNA may comprise one or more selectable markers or one or more site specific recombination sites. In one aspect the vector comprises 2 or more, such as 3, heterospecific and incompatible site specific recombination sites. In one aspect the site specific recombination sites may be loxP sites, or variants thereof, or FRT sites or variants thereof. In one aspect the vector comprises one or more transposon ITR (inverted terminal repeat) sequences.

In one aspect the non-human animals of the invention suitably do not produce any fully humanised antibodies. In one aspect this is because there is no DNA inserted from the human constant region. Alternatively there is no human constant region DNA in the genome capable of forming an antibody in conjunction with the inserted human variable region DNA component, for example due to mutation within any human constant region DNA or distance from any constant region human DNA and human variable region DNA.

In one aspect human light chain constant region DNA may be included in the cell genome, such that a fully human lambda or kappa human antibody chain might be generated, but this would only be able to form an antibody with a chimaeric heavy chain, and not produce a fully human antibody having human variable and constant regions.

In one aspect the non-human mammal genome is modified to prevent expression of fully host-species specific antibodies. Fully host species specific antibodies are antibodies that have both variable and constant regions from the host organism. In this context the term ‘specific’ is not intended to relate to the binding of the antibodies produced by the cells or animals of the invention but rather to the origin of the DNA which encodes those antibodies.

In one aspect the non-human mammal genome is modified to prevent expression of the native (fully host species specific) antibodies in the mammal by inactivation of all or a part of the host non-human mammal Ig loci. In this context, inactivation or prevention of endogenous antibody or gene segment usage (using any inactivation technique described herein) is, for example, substantially complete inactivation or prevention (substantially 100%, ie, essentially none (eg, less than 10, 5, 4, 3, 2, 1 or 0.5%) of the endogenous antibody chain (eg, no endogenous heavy chains) is expressed). This can be determined, for example, at the antibody chain (protein) level by assessing the antibody repertoire produced by the non-human vertebrate, mammal or at the nucleotide level by assessing mRNA transcripts of antibody chain loci, eg, using RACE. In an embodiment, inactivation is more than 50% (ie, 50% or less of the antibodies or transcripts are of an endogenous antibody chain), 60%, 70%, 80%, 85%, 90%, 95%, 96%, 97%, 98% or 99%. For example, in an embodiment, endogenous heavy chain expression is substantially inactivated such that no more than 85%, 90%, 95%, 96%, 97%, 98% or 99% of the heavy chain repertoire of the vertebrate (mammal) is provided by endogenous heavy chains. For example, endogenous heavy chain expression is substantially inactivated such that substantially none of the heavy chain repertoire of the vertebrate (mammal) is provided by endogenous heavy chains. For example, in an embodiment, endogenous heavy chain expression is substantially inactivated such that no more than 85%, 90%, 95%, 96%, 97%, 98% or 99% of the kappa chain repertoire of the vertebrate (mammal) is provided by endogenous kappa chains. For example, endogenous kappa chain expression is substantially inactivated such that substantially none of the kappa chain repertoire of the vertebrate (mammal) is provided by endogenous kappa chains. For example, in an embodiment, endogenous heavy chain expression is substantially inactivated such that no more than 85%, 90%, 95%, 96%, 97%, 98% or 99% of the lambda chain repertoire of the vertebrate (mammal) is provided by endogenous lambda chains. For example, endogenous lambda chain expression is substantially inactivated such that substantially none of the lambda chain repertoire of the vertebrate (mammal) is provided by endogenous lambda chains.

In one aspect this is achieved by inversion of all or part of the non-human mammal VDJ region, or VJ region, optionally by insertion of one or more site specific recombinase sites into the genome and then use of these sites in recombinase-mediated excision or inversion of all or a part of the non-human mammal Ig locus. In one aspect a double inversion, may be employed, the first to move the V(D)Js away from the endogenous locus and then a more local inversion which puts them in the correct orientation. In one aspect a single IoxP site is used to invert the non-human mammal VDJ region to a centromeric locus or telomeric locus.

In one example, a mouse or mouse cell of the invention comprises inverted endogenous heavy chain gene segments (eg, VH, D and JH, such as the entire endogenous heavy chain VDJ region) that are immediately 3′ of position 119753123, 119659458 or 120918606 on an endogenous mouse chromosome 12. Optionally, the genome of the mouse or cell is homozygous for said chromosome 12.

The Invention Also Provides:

A cassette for inversion and inactivation of endogenous non-human vertebrate (eg, mouse or rat) antibody chain gene segments, the segments being part of an antibody chain locus sequence on a chromosome of a non-human vertebrate (eg, mouse or rat) cell (eg, ES cell) wherein the sequence is flanked at its 3′ end by a site-specific recombination site (eg, lox, rox or frt), the cassette comprising a nucleotide sequence encoding an expressible label or selectable marker and a compatible site-specific recombination site (eg, lox, rox or frt) flanked by a 5′ and a 3′ homology arm, wherein the homology arms correspond to or are homologous to adjacent stretches of sequence in the cell genome on a different chromosome or on said chromosome at least 10, 15, 20, 25, 30, 35, 40, 45 or 50 mb away from the endogenous gene segments.

The Invention Also Provides:

A cassette for inversion and inactivation of endogenous mouse antibody heavy chain gene segments, the segments being part of a heavy chain locus sequence on chromosome 12 of a mouse cell (eg, ES cell) wherein the sequence is flanked at its 3′ end by a site-specific recombination site (eg, lox, rox or frt), the cassette comprising a nucleotide sequence encoding an expressible label or selectable marker and a compatible site-specific recombination site (eg, lox, rox or frt) flanked by a 5′ and a 3′ homology arm, wherein the homology arms correspond to or are homologous to adjacent stretches of sequence in the mouse cell genome on a different chromosome or on chromosome 12 at least 10, 15, 20, 25, 30, 35, 40, 45 or 50 mb away from the endogenous gene segments.

The Invention Provides:

A cassette for inversion and inactivation of endogenous mouse antibody heavy chain gene segments, the segments being part of a heavy chain locus sequence on chromosome 12 of a mouse cell (eg, ES cell) wherein the sequence is flanked at its 3′ end by a site-specific recombination site (eg, lox, rox or frt), the cassette comprising a nucleotide sequence encoding an expressible label or selectable marker and a compatible site-specific recombination site (eg, lox, rox or fa) flanked by a 5′ and a 3′ homology arm, wherein (i) the 5′ homology arm is mouse chromosome 12 DNA from coordinate 119753124 to coordinate 119757104 and the 3′ homology arm is mouse chromosome 12 DNA from coordinate 119749288 to 119753123; or (ii) the 5′ homology arm is mouse chromosome 12 DNA from coordinate 119659459 to coordinate 119663126 and the 3′ homology arm is mouse chromosome 12 DNA from coordinate 119656536 to 119659458; or (iii) the 5′ homology arm is mouse chromosome 12 DNA from coordinate 120918607 to coordinate 120921930 and the 3′ homology arm is mouse chromosome 12 DNA from coordinate 120915475 to 120918606.
Embodiment (i) results in an inversion of mouse chromosome 12 from coordinate 119753123 to coordinate 114666436.
Embodiment (ii) results in an inversion of mouse chromosome 12 from coordinate 119659458 to coordinate 114666436
Embodiment (iii) results in an inversion of mouse chromosome 12 from coordinate 12091806 to coordinate 114666436.

Thus, the invention provides a mouse or mouse cell whose genome comprises an inversion of a chromosome 12, wherein the inversion comprises inverted endogenous heavy chain gene segments (eg, VH, D and JH, such as the entire endogenous heavy chain VDJ region); wherein the mouse comprises a transgenic heavy chain locus comprising a plurality of human VH gene segments, a plurality of human D segments and a plurality of human JH segments operably connected upstream of an endogenous constant region (eg, C mu) so that the mouse or cell (optionally following differentiation into a B-cell) is capable of expressing an antibody comprising a variable region comprising sequences derived from the human gene segments; and wherein the inversion is (i) an inversion of mouse chromosome 12 from coordinate 119753123 to coordinate 114666436; (ii) an inversion of mouse chromosome 12 from coordinate 119659458 to coordinate 114666436; or (iii) an inversion of mouse chromosome 12 from coordinate 12091806 to coordinate 114666436.

In one embodiment, the endogenous gene segments are from a 129-derived mouse cell (eg, segments from an AB2.1 cell) and the homology arms are isogenic DNA (ie, identical to 129-derived endogenous sequences demarcated by the respective coordinates stated in (i) to (iii) above). Thus, no new sequence is created by homologous recombination using these homology arms. In another embodiment, the arms are from a mouse strain that is different from the endogenous strain. The site-specific recombination sites are mutually compatible and mutually inverted such that, on expression of an associated recombinase enzyme (eg, Cre, Dre or Flp), recombination between the site in the inserted inversion cassette and the site flanking the endogenous gene segments is carried out, thereby inverting and moving the endogenous gene segments far upstream (5′) of their original location in the heavy chain locus. This inactivates endogenous heavy chain expression. Similarly, light chain inactivation can be performed by choosing the homology arms of the inversion cassette with reference to a chromosomal region spaced at least 10, 15, 20, 25, 30, 35, 40, 45 or 50 mb away from the endogenous light chain locus, the latter comprising a site-specific recombination site that is compatible with the site in the inversion cassette.

In one embodiment, the expressible label is a fluorescent label, eg, GFP or a variant thereof (eg, YFP, CFP or RFP). Thus, a label is used instead of a selection marker, such as one that confers resistance to allow for selection of transformants.

The invention provides a method of inactivating gene segments of an endogenous antibody locus, the method comprising
(i) Providing a non-human vertebrate cell (eg, an ES cell, eg, a mouse ES cell) whose genome comprises an antibody chain locus comprising endogenous variable region gene segments;
(ii) Targeting a site-specific recombination site to flank the 3′ of the 3′-most of said endogenous gene segments;
(iii) Targeting a second site-specific recombination site at least 10mb away from said endogenous gene segments, the second site being compatible with the first site inverted with respect to the first site;
(iv) Expressing a recombinase compatible with said sites to effect site-specific recombination between said sites, thereby inverting and moving said gene segments away from said locus, wherein the endogenous gene segments are inactivated; and
(v) Optionally developing the cell into a progeny cell or vertebrate (eg, mouse or rat) whose genome is homozygous for the inversion.

The genome of the progeny cell or vertebrate can comprise transgenic heavy and/or light chain loci, each capable of expressing antibody chains comprising human variable regions. Optionally, endogenous heavy and kappa light chain expression is inactivated by inverting endogenous heavy and kappa variable region gene segments according to the method of the invention. Optionally, endogenous lambda chain expression is also inactivated in this way.

In an alternative to the method and inversion cassettes of the invention, instead of inverting and moving variable region gene segments only, other parts of the endogenous locus can alternatively or additionally be inverted and moved to effect inactivation. For example, one or more endogenous regulatory elements (eg, Smu and/or Emu) and/or one or more endogenous constant regions (eg, Cmu and/or Cgamma) can be inverted and moved.

Sites that “flank” in the above contexts of the invention can be provided such that a site-specific recombination site immediately flanks the endogenous sequence or is spaced therefrom, eg, by no more than 250, 200, 250, 100, 50 or 20 kb in the 3′ direction.

In one aspect the non-human mammal genome into which human DNA is inserted comprises endogenous V, (D) and J regions, and the endogenous sequences have not been deleted.

The invention comprises a method for insertion of multiple DNA fragments into a DNA target, suitably to form a contiguous insertion in which the inserted fragments are joined together directly without intervening sequences. The method is especially applicable to the insertion of a large DNA fragment into a host chromosome which can be carried out in a stepwise fashion.

In one aspect the method comprises insertion of a first DNA sequence into a target, the sequence having a DNA vector portion and a first sequence of interest (X1); insertion of a second DNA sequence into the vector portion of the first sequence, the second DNA sequence having a second sequence of interest (X2) and a second vector portion; and then excising any vector sequence DNA separating X1 and X2 to provide a contiguous X1X2, or X2X1 sequence within the target. There is optionally insertion of a further one or more DNA sequences, each DNA sequence having a further sequence of interest (X3, . . . ) and a further vector portion, into the vector portion of the preceding DNA sequence, to build up a contiguous DNA fragment in the target.

The DNA target for insertion of the first DNA sequence may be a specific site or any point in the genome of a particular cell.

The general method is described herein in relation to the insertion of elements of the human VDJ region, but is applicable to insertion of any DNA region, from any organism, and in particular insertion of large DNA fragments of >100 kB, such as 100-250 kb, or even larger, such as that of the TCR or HLA. Features and approaches described herein in respect of the VDJ insertion may be equally applied to the any of the methods disclosed

In one aspect the inserted DNA is human DNA, such as the human VDJ or VJ region, is built up in the genome of a cell, such as an ES cell, in a stepwise manner using 2, 3, 4, 5, 6, 7, 8, 9, 10, 11, 12, 13, 14, 15, 20 or more separate insertions for each heavy chain or light chain region. Fragments are suitably inserted at the same or substantially the same cell locus, e.g. ES cell locus, one after another, to form the complete VDJ or VJ region, or part thereof. The present invention also relates to cells and non-human animals comprising intermediates in the process whose genomes may comprise only a partial VDJ region, such as only human variable region DNA.

In a further aspect the method for producing a transgenic non-human mammal comprises the insertion of human VDJ or VJ regions upstream of the host non-human mammal constant region by step-wise insertion of multiple fragments by homologous recombination, preferably using an iterative process. Suitably fragments of approximately 100 KB from the human VDJ and VJ locus are inserted, suitably to form part of, or a complete, VDJ or VJ region after the final iteration of the insertion process, as disclosed herein.

In one aspect the insertion process commences at a site where an initiation cassette has been inserted into the genome of a cell, such as an ES cell, providing a unique targeting region. In one aspect the initiation cassette is inserted in the non-human mammal heavy chain locus, for use in insertion of human heavy chain DNA. Similarly an initiation cassette may be inserted in the non-human mammal light chain locus, for use in insertion of human light chain VJ DNA The initiation cassette suitably comprises a vector backbone sequence with which a vector having a human DNA fragment in the same backbone sequence can recombine to insert the human DNA into the cell (e.g. ES) cell genome, and suitably a selection marker, such as a negative selection marker. Suitably the vector backbone sequence is that of a BAC library, to allow BACs to be used in the construction of the ES cells and mammals. The vector backbone sequence may however be any sequence which serves as a target site into which a homologous sequence can insert, for example by homologous recombination, for example RMCE, and is preferably not DNA encoding any of the VDJ or constant region.

In one aspect the insertion of the first DNA fragment into an initiation cassette is followed by insertion of a second DNA fragment into a portion of the first DNA fragment, suitably a part of the vector backbone of the second DNA fragment. In one aspect an inserted DNA fragment comprises a part of the human VDJ region flanked by 5′ and/or 3′ sequences that are not from the human VDJ region. In one aspect the 5′ and/or 3′ flanking sequences may each contain one or more selectable markers, or be capable of creating a selectable system once inserted into the genome. In one aspect one or both flanking sequences may be removed from the genome in vitro, or in vivo, following insertion. In one aspect the method comprises insertion of a DNA fragment followed by selection of both 5′ and 3′ ends of the inserted fragment flanking the human VDJ DNA. In one aspect the iterative insertion is made by insertion of DNA fragments at the 5′ end of the previous inserted fragment, and in this aspect there may be deletion in vivo of the vector DNA which separates the inserted human DNA sequences, to provide a contiguous human DNA sequence.

In one aspect insertion of human VDJ DNA into a genome may be achieved without leaving any flanking DNA in the genome, for example by transposase mediate DNA excision. One suitable transposase is the Piggybac transposase.

In one aspect the first human variable region fragment is inserted by homologous recombination at the initiation cassette backbone sequence and then the DNA of any negative selection marker and initiation cassette are subsequently removed by recombination between recombinase target sequences, such as FRT using in this example, FLPase expression. Generally repeated targeted insertions at the (e.g. BAC) backbone initiation sequence and subsequent removal by rearrangement between recombinase target sequences are repeated to build up the entire human VDJ region upstream of the host non-mammal constant region.

In one aspect a selectable marker or system may be used in the method. The marker may be generated upon insertion of a DNA fragment into a genome, for example forming a selectable marker in conjunction with a DNA element already present in the genome.

In one aspect the cell (e.g. ES) cell genome does not contain 2 identical selectable markers at the same time during the process. It can be seen that the iterative process of insertion and selection can be carried out using only 2 different selection markers, as disclosed in the examples herein, and for example the third selectable marker may be identical to the first marker, as by the time of insertion of the third vector fragment the first vector fragment and the first marker has been removed.

In one aspect a correct insertion event, is confirmed before moving to the next step of any multistep cloning process, for example by confirmation of BAC structure using high density genomic arrays to screen ES cells to identify those with intact BAC insertions, sequencing and PCR verification.

Initiation Cassette (Also Called a “Landing Pad”)

The invention also relates to a polynucleotide ‘landing pad’ sequence, the polynucleotide comprising nucleic acid regions homologous to regions of a target chromosome to allow for insertion by homologous recombination into the target chromosome, and comprising a nucleic acid site which permits recombinase-driven insertion of nucleic acid into the landing pad. The invention also relates to vectors, cells and mammals of the invention comprising a landing pad as disclosed herein inserted into the genome of the cell.

The landing pad optionally comprises a non-endogenous S-mu, e.g. a rat S-mu switch

The landing pad optionally comprises (in 5′ to 3′ orientation) a mouse Ep sequence, a non-human, non-mouse (e.g. rat) Switch p and at least a portion of a mouse Cμ or the entire mouse Cp.

The rat switch sequence optionally comprises or consists of SEQ ID NO 1 The landing pad optionally comprises the 5′ homology arm of SEQ ID NO 6.

The landing pad optionally has the sequence of SEQ ID 2 or SEQ ID NO 3.

In one embodiment, the landing pad comprises an expressible label. For example the label is a fluorescent label, eg, GFP or a variant thereof (eg, YFP, CFP or RFP). Thus, a label is used instead of a selection marker (such as one that confers resistance to allow for selection of transformants).

In an embodiment, the landing pad comprises 5′ and 3′ homology arms for insertion into the cell genome using homologous recombination. The homology arms can be isogenic DNA (eg, identical to 129-derived endogenous sequences of when a 129-derived ES cell is used). Thus, no new sequence is created by homologous recombination using these homology arms. In another embodiment, the arms are from a mouse strain that is different from the endogenous strain (ES cell strain),

The methods of the invention include methods wherein the landing pad sequence comprises any of the configurations or sequences as disclosed herein.

Another method of the invention comprises the step of insertion of the landing pad into a mouse chromosome by homologous recombination between mouse J1-4 and mouse C mu sequences.

Another method of the invention comprises the step of insertion of the landing pad into the mouse chromosome 12 by homologous recombination between mouse J1-4 and E mu.

In one aspect the method uses site specific recombination for insertion of one or more vectors into the genome of a cell, such as an ES cell. Site specific recombinase systems are well known in the art and may include Cre-lox, and FLP/FRT or combinations thereof, in which recombination occurs between 2 sites having sequence homology.

Additionally or alternatively to any particular CreiLox or FLPIFRT system described herein, other recombinases and sites that may be used in the present invention include Die recombinase, rox sites, and PhiC31 recombinase.

Suitable BACs are available from the Sanger centre, see “A genome-wide, end-sequenced 1295v BAC library resource for targeting vector construction”. Adams D J, Quail M A, Cox T, van der Weyden L, Gorick B D, Su Q, Chan W I, Davies R, Bonfield J K, Law F, Humphray S, Plumb B, Liu P, Rogers J, Bradley A. Genomics. 2005 December; 86(6):753-8. Epub 2005 Oct 27. The Wellcome Trust Sanger Institute, Hinxton, Cambridgeshire CB10 1SA, UK. BACs containing human DNA are also available from, for example, lnvitrogen™. A suitable library is described in Osoegawa K et af, Genome Research 2001. 11: 483-496.

In one aspect a method of the invention specifically comprises:
(1) insertion of a first DNA fragment into a non-human ES cell, the fragment containing a first portion of human VDJ or VJ region DNA and a first vector portion containing a first selectable marker;
(2) optionally deletion of the a part of the first vector portion;
(3) insertion of a second DNA fragment into a non-human ES cell containing the first DNA fragment, the insertion occurring within the first vector portion, the second DNA fragment containing a second portion of the human VDJ or VJ region and a second vector portion containing a second selectable marker,
(4) deletion of the first selectable marker and first vector portion, preferably by a recombinase enzyme action;
(5) insertion of a third DNA fragment into a non-human ES cell containing the second DNA fragment, the insertion occurring within the second vector portion, the third DNA fragment containing a third portion of the human VDJ or VJ region and a third vector portion containing third selectable marker,
(6) deletion of the second selectable marker and second vector portion; and
(7) iteration of the steps of insertion and deletion, as necessary, for fourth and further fragments of the human VDJ or VJ human regions, as necessary, to produce an ES cell with a part or all of the human VDJ or VJ region inserted as disclosed herein, and suitably to remove all the vector portions within the ES cell genome.

In another aspect the invention comprises
(1) insertion of DNA forming an initiation cassette into the genome of a cell;
(2) insertion of a first DNA fragment into the initiation cassette, the first DNA fragment comprising a first portion of a human DNA and a first vector portion containing a first selectable marker or generating a selectable marker upon insertion;
(3) optionally removal of part of the vector DNA
(4) insertion of a second DNA fragment into the vector portion of the first DNA fragment, the second DNA fragment containing a second portion of human DNA and a second vector portion, the second vector portion containing a second selectable marker, or generating a second selectable marker upon insertion;
(5) optionally, removal of any vector DNA to allow the first and second human DNA fragments to form a contiguous sequence; and
(6) iteration of the steps of insertion of human VDJ DNA and vector DNA removal, as necessary, to produce a cell with all or part of the human VDJ or VJ region sufficient to be capable of generating a chimaeric antibody in conjunction with a host constant region, wherein the insertion of one, or more, or all of the DNA fragments uses site specific recombination.

In one aspect the non-human mammal is able to generate a diversity of at least 1×10⁶ different functional chimaeric immunoglobulin sequence combinations.

In one aspect the targeting is carried out in ES cells derived from the mouse C57BL16N, C57BL/6J, 129S5 or 129Sv strain.

In one aspect non-human animals, such as mice, are generated in a RAG-1-deficient or a RAG-2-deficient background, or other suitable genetic background which prevents the production of mature host B and T lymphocytes.

In one aspect the non-human mammal is a rodent, suitably a mouse, and cells of the invention. are rodent cells or ES cells, suitably mouse ES cells.

The ES cells of the present invention can be used to generate animals using techniques well known in the art, which comprise injection of the ES cell into a blastocyst followed by implantation of chimaeric blastocystys into females to produce offspring which can be bred and selected for homozygous recombinants having the required insertion. In one aspect the invention relates to a chimeric animal comprised of ES cell-derived tissue and host embryo derived tissue. In one aspect the invention relates to genetically-altered subsequent generation animals, which include animals having a homozygous recombinants for the VDJ and/or VJ regions.

In a further aspect the invention relates to a method for producing an antibody specific to a desired antigen the method comprising immunizing a transgenic non-human mammal as above with the desired antigen and recovering the antibody (see e.g. Harlow, E. & Lane, D. 1998, 5th edition, Antibodies: A Laboratory Manual, Cold Spring Harbor Lab. Press, Plainview, NY; and Pasqualini and Arap, Proceedings of the National Academy of Sciences (2004) 101:257-259). Suitably an immunogenic amount of the antigen is delivered. The invention also relates to a method for detecting a target antigen comprising detecting an antibody produced as above with a secondary detection agent which recognises a portion of that antibody.

In a further aspect the invention relates to a method for producing a fully humanised antibody comprising immunizing a transgenic non-human mammal as above with the desired antigen, recovering the antibody or cells expressing the antibody, and then replacing the non-human mammal constant region with a human constant region. This can be done by standard cloning techniques at the DNA level to replace the non-human mammal constant region with an appropriate human constant region DNA sequence—see e.g. Sambrook, J and Russell, D. (2001, 3′d edition) Molecular Cloning: A Laboratory Manual (Cold Spring Harbor Lab. Press, Plainview, NY).

In a further aspect the invention relates to humanised antibodies and antibody chains produced according to the present invention, both in chimaeric and fully humanised form, and use of said antibodies in medicine. The invention also relates to a pharmaceutical composition comprising such an antibodies and a pharmaceutically acceptable carrier or other excipient.

Antibody chains containing human sequences, such as chimaeric human—non-human antibody chains, are considered humanised herein by virtue of the presence of the human protein coding regions region. Fully humanised antibodies may be produced starting from DNA encoding a chimaeric antibody chain of the invention using standard techniques.

Methods for the generation of both monoclonal and polyclonal antibodies are well known in the art, and the present invention relates to both polyclonal and monoclonal antibodies of chimaeric or fully humanised antibodies produced in response to antigen challenge in non-human mammals of the present invention.

In a yet further aspect, chimaeric antibodies or antibody chains generated in the present invention may be manipulated, suitably at the DNA level, to generate molecules with antibody-like properties or structure, such as a human variable region from a heavy or light chain absent a constant region, for example a domain antibody; or a human variable region with any constant region from either heavy or light chain from the same or different species; or a human variable region with a non-naturally occurring constant region; or human variable region together with any other fusion partner. The invention relates to all such chimaeric antibody derivatives derived from chimaeric antibodies identified according to the present invention.

In a further aspect, the invention relates to use of animals of the present invention in the analysis of the likely effects of drugs and vaccines in the context of a quasi-human antibody repertoire.

The invention also relates to a method for identification or validation of a drug or vaccine, the method comprising delivering the vaccine or drug to a mammal of the invention and monitoring one or more of: the immune response, the safety profile; the effect on disease.

The invention also relates to a kit comprising an antibody or antibody derivative as disclosed herein and either instructions for use of such antibody or a suitable laboratory reagent, such as a buffer, antibody detection reagent.

The invention also relates to a method for making an antibody, or part thereof, the method comprising providing:
(i) a nucleic acid encoding an antibody, or a part thereof, obtained according to the present invention; or
(ii) sequence information from which a nucleic acid encoding an antibody obtained according to the present invention, or part thereof, can be expressed to allow an antibody to be produced.

The present invention also relates to a chimaeric antibody comprising a human variable region and a non-human vertebrate or mammal (optionally a rat or mouse) constant region (optionally a C gamma or C mu), wherein the antibody is encoded by a nucleotide sequence corresponding to the nucleotide sequence of a chimaeric heavy chain locus of a cell (optionally a B-cell, ES cell or hybridoma), the locus comprising a non-human vertebrate constant region nucleotide sequence and a rearranged VDJ nucleotide sequence produced by the in vivo rearrangement of a human V region, a human D region and a human J region, the V region being selected from one of a V1-3 region, V2-5 region, V4-4 region, V1-2 region or V6-1 region, and optionally a V1-3 or V6-1 segment. Optionally, the J region is any of JH1, JH2, JH3, JH4, JH5 or JH6, and in one aspect is JH4 or JH6. The D region is, in one aspect, any D3-9, D3-10, D6-13 or D6-19. In one example, rearranged VDJ nucleotide sequence is produced by the in vivo rearrangement of human V1-3 and JH4 (optionally with D3-9, D3-10, D6-13 or D-19); or V1-3 and JH6 (optionally with D3-9, D3-10, D6-13 or D-19); or V6-1 and JH4 (optionally with D3-9, D3-10, D6-13 or D-19); or V6-1 and JH6 (optionally with D3-9, D3-10, D6-13 or D-19). In one example the rearranged VDJ nucleotide sequence is produced by the in vivo rearrangement of human V6-1 DH3-10, V1-3 DH3-10, V1-3 DH6-19, V1-3 Dh3-9 or V6-1 DH6-19. In one aspect the antibody comprises any combination exemplified in the Examples and Figures herein. Optionally, the in vivo rearrangement is in a cell (eg, B cell or ES cell) derived from the same non-human vertebrate species as the constant region sequence (eg, a mouse B cell or ES cell). The invention also relates to a non-human vertebrate or mammal cell (eg, a B-cell or ES cell or hybridoma) whose genome comprises a chimaeric heavy chain locus as described above in this paragraph. The invention also relates to a non-human vertebrate or mammal (eg, a mouse or rat) whose genome comprises a chimaeric heavy chain locus as described above in this paragraph.

The present invention also relates to a non-human vertebrate or mammal having a genome encoding a chimaeric antibody, the chimaeric antibody comprising a human variable region and a non-human vertebrate or mammal (optionally a rat or mouse) constant region (optionally a C gamma or C mu), the mammal:
expressing more V1-3 antibodies than V2-5, V4-4, V1-2 or V6-1 antibodies; and/or
expressing more V1-3 JH4 or V1-3 JH6 antibodies than any of, individually, V1-3 JH1, V1-3 JH2, V1-3 JH3 or V1-3 JH5 antibodies, and/or
expressing more V6-1 JH4 or V6-1 JH6 antibodies than any of, individually, V6-1 JH1, V6-1 JH2, V6-1 JH3 or V6-1 JH5 antibodies and/or
expressing a greater number of V1-3 DH3-10 antibodies than antibodies V1-3 with any other D region. Expression of antibodies can be assessed by methods readily available to the skilled person and as conventional in the art. For example, expression can be assessed at the mRNA level as shown in the examples below.

The invention also relates to a chimaeric antibody comprising a human variable region and a non-human vertebrate or mammal (optionally a rat or mouse) constant region (optionally a light chain constant region), wherein the antibody is obtainable from a mammal (optionally a rat or mouse) whose genome comprises an antibody chain locus comprising a germline human kappa V1-8 and germline human kappa J1 sequence, and wherein the antibody is obtainable by in vivo recombination in said mammal of the V1-8 and J1 sequences and wherein the antibody has a variable region sequence which is different from that which is encoded by germline human kappa V1-8 and germline human kappa J1 sequences. Thus, in this aspect of the invention the human germline sequences are able to undergo productive rearrangement to form a coding sequence which, in conjunction with the non-human constant region sequence, can be expressed as a chimaeric antibody chain having at least a complete human variable region and a non-human constant region. This is in contrast (as the examples show below) to the combination of the germline human kappa V1-8 and germline human kappa J1 sequences per se, which do not provide for an antibody coding sequence (due to the inclusion of stop codons). In one aspect the rearranged sequence of the chimaeric antibody is a result of somatic hypermutation. In one aspect the antibody is a kappa antibody; in another aspect the antibody comprises a non-human heavy chain constant region (eg, a rat or mouse C gamma or C mu). The antibody sequence optionally comprises a X₁X₂ T F G Q, where X₁X₂=PR, RT, or PW (SEQ ID No 21); optionally a X₁X₂ T F G Q GT K V E I KR ADA (SEQ ID No 22) motif. Such motifs are not found in the equivalent position in the germline sequence as shown in the examples. The invention also relates to a non-human vertebrate or mammal cell (eg, a B-cell or ES cell or hybridoma) whose genome comprises a chimaeric antibody chain locus as described above in this paragraph. The invention also relates to a non-human vertebrate or mammal (eg, a mouse or rat) whose genome comprises a chimaeric antibody chain locus as described above in this paragraph.

The invention also relates to a chimaeric antibody comprising a human variable region and a non-human vertebrate or mammal (optionally a rat or mouse) constant region (optionally a light chain constant region), wherein the antibody is obtainable from a mammal (optionally a rat or mouse) whose genome comprises an antibody chain locus comprising a germline human kappa V1-6 and germline human kappa J1 sequence, and wherein the antibody is obtainable by in vivo recombination in said mammal of the V1-6 and J1 sequences and wherein the antibody has a variable region sequence which is different from that which is encoded by germline human kappa V1-6 and germline human kappa J1 sequences. Thus, in this aspect of the invention the human germline sequences are able to undergo productive rearrangement to form a coding sequence which, in conjunction with the non-human constant region sequence, can be expressed as a chimaeric antibody chain having at least a complete human variable region and a non-human constant region. This is in contrast (as the examples show below) to the combination of the germline human kappa V1-6 and germline human kappa J1 sequences per se, which do not provide for an antibody coding sequence (due to the inclusion of stop codons). In one aspect the rearranged sequence of the chimaeric antibody is a result of somatic hypermutation. In one aspect the antibody is a kappa antibody; in another aspect the antibody comprises a non-human heavy chain constant region (eg, a rat or mouse C gamma or C mu). The antibody sequence optionally comprises a X₃X₄T F G Q, where X₃X₄=PR or PW (SEQ ID No 23); optionally a X₃X₄T F G Q G T K V E I K R A D A (SEQ ID No 24) motif. Such motifs are not found in the equivalent position in the germline sequence as shown in the examples. The invention also relates to a non-human vertebrate or mammal cell (eg, a B-cell or ES cell or hybridoma) whose genome comprises a chimaeric antibody chain locus as described above in this paragraph. The invention also relates to a non-human vertebrate or mammal (eg, a mouse or rat) whose genome comprises a chimaeric antibody chain locus as described above in this paragraph.

The invention also relates to a chimaeric antibody comprising a human variable region and a non-human (optionally a rat or mouse) constant region (optionally a C gamma or C mu or a C kappa), wherein the antibody is obtainable from a mammal (optionally a rat or mouse) whose genome comprises an antibody chain locus comprising a germline human kappa V1-5 and germline human kappa J1 sequence, and wherein the antibody is obtainable by in vivo recombination in said mammal of the V1-5 and J1 sequences. The invention also relates to a non-human vertebrate or mammal cell (eg, a B-cell or ES cell or hybridoma) whose genome comprises a chimaeric antibody chain locus as described above in this paragraph. The invention also relates to a non-human vertebrate or mammal (eg, a mouse or rat) whose genome comprises a chimaeric antibody chain locus as described above in this paragraph.

The invention also relates to a chimaeric antibody comprising a human variable region and a non-human (optionally a rat or mouse) constant region (optionally a C gamma or C mu or a C kappa), wherein the antibody is obtainable from a mammal (optionally a rat or mouse) whose genome comprises an antibody chain locus comprising a germline human kappa V1-5 and germline human kappa J4 sequence, and wherein the antibody is obtainable by in vivo recombination in said mammal of the V1-5 and J4 sequences. The invention also relates to a non-human vertebrate or mammal cell (eg, a B-cell or ES cell or hybridoma) whose genome comprises a chimaeric antibody chain locus as described above in this paragraph. The invention also relates to a non-human vertebrate or mammal (eg, a mouse or rat) whose genome comprises a chimaeric antibody chain locus as described above in this paragraph.

Antibodies of the invention may be isolated, in one aspect being isolated from the cell or organism in which they are expressed.

A non-human mammal whose genome comprises:
(a) the human IgH VDJ region upstream of the host non-human mammal constant region; and
(b) the human Ig light chain kappa V and J regions upstream of the host non-human mammal kappa constant region and/or the human Ig light chain lambda V and J regions upstream of the host non-human mammal lambda constant region;
wherein the non-human mammal is able to produce a repertoire of chimaeric antibodies having a non-human mammal constant region and a human variable region, and optionally wherein the non-human mammal genome is modified to prevent expression of fully host-species specific antibodies.

A non-human mammal ES cell whose genome comprises:
(a) the human IgH V, D and J region upstream of a non-human mammal constant region; and
(b) the human Ig locus light chain kappa V and J regions upstream of the host non-human mammal kappa constant region, and/or the human Ig locus light chain lambda V and J regions upstream of the host non-human mammal lambda constant region

wherein the ES cell is capable of developing into a non-human mammal, being able to produce a repertoire of antibodies which are chimaeric, having a non-human mammal constant region and a human variable region.

A method for producing a transgenic non-human mammal able to produce a repertoire of chimaeric antibodies, the antibodies having a non-human mammal constant region and a human variable region, the method comprising inserting by homologous recombination into a non-human mammal ES cell genome
(a) the human IgH VDJ region upstream of the host non-human mammal heavy chain constant region, and
(b) the human IgL VJ region for lambda or kappa chains upstream of the host non-human mammal lambda or kappa chain constant region, respectively

such that the non-human mammal is able to produce a repertoire of chimaeric antibodies having a non-human mammal constant region and a human variable region, wherein steps (a) and (b) can be carried out in either order and each of steps (a) and (b) can be carried out in a stepwise manner or as a single step.

In one aspect the insertion of human VDJ or VJ regions upstream of the host non-human mammal constant region is accomplished by step-wise insertion of multiple fragments by homologous recombination.

In one aspect the step-wise insertions commence at a site where an initiation cassette has been inserted into the genome of an ES cell providing a unique targeting region consisting of a BAC backbone sequence and a negative selection marker.

In one aspect the first human variable region fragment is inserted by homologous recombination at the initiation cassette BAC backbone sequence and said negative selection marker and initiation cassette are subsequently removed by recombination between recombinase target sequences.

In one aspect repeated targeted insertions at the BAC backbone initiation sequence and subsequent removal of the backbone by rearrangement between recombinase target sequences is repeated to build up the entire human VDJ region upstream of the host non-mammal constant region.

Insertion of Human Variable Region Gene Segments Precisely within the Endogenous Mouse JH4-Cmu intron

There is further provided a cell or non human mammal according to the invention wherein the mammal is a mouse or the cell is a mouse cell and wherein the insertion of the human heavy chain DNA is made in a mouse genome between coordinates 114,667,091 and 114,665,190 of mouse chromosome 12.

There is further provided a cell or non human mammal according to the invention wherein the insertion of the human heavy chain DNA is made at coordinate 114,667,091.

There is further provided a cell or non human mammal according to the invention wherein the human IgH VDJ region comprises nucleotides 105,400,051 to 106,368,585 from human chromosome 14 (coordinates refer to NCBI36 for the human genome).

There is further provided a method, cell or non human mammal according to the invention wherein a human coding region DNA sequence is in a functional arrangement with a non-human mammal control sequence, such that transcription of the human DNA is controlled by the non-human mammal control sequence. In one example, the initiation cassette is inserted between the mouse J4 and C alpha exons. There is further provided an initiation cassette suitable for use in the method comprising a vector backbone sequence and a selection marker.

The Invention Provides the Following Aspects (Starting at Aspect Number 103):
103. A cell or non human mammal according to any one of the above configurations, examples, embodiments or aspects, wherein the mammal is a mouse or the cell is a mouse cell and wherein the insertion of the human heavy chain DNA is made in a mouse genome between coordinates 114,667,091 and 114,665,190 of mouse chromosome 12.
104. A cell or non human mammal according to any one of the above configurations, examples, embodiments or aspects, wherein the insertion of the human heavy chain DNA is made at coordinate 114,667,091.
105. A cell or mammal according to any one of the above configurations, examples, embodiments or aspects, wherein the human IgH VDJ region comprises nucleotides 105,400,051 to 106,368,585 from human chromosome 14 (coordinates refer to NCBI36 for the human genome).
106. A method, cell or mammal according to any one of the above configurations, examples, embodiments or aspects, wherein a human coding region DNA sequence is in a functional arrangement with a non-human mammal control sequence, such that transcription of the human DNA is controlled by the non-human mammal control sequence.
107. A method according to aspect 106 wherein the initiation cassette is inserted between the mouse J4 and C alpha exons.
108. An initiation cassette suitable for use in the method of aspect 107 comprising a vector backbone sequence and a selection marker.

Inactivation of Endogenous Antibody Chain Expression by Insertion of Human Antibody Variable Region Gene Segments
109. A non-human vertebrate (optionally a mouse or rat) or non-human vertebrate cell (optionally a mouse or rat cell) having a genome that
   (i) comprises a transgenic antibody chain locus capable of expressing an antibody chain comprising a human variable region (optionally following antibody gene rearrangement); and
   (ii) is inactivated for endogenous non-human vertebrate antibody chain expression; wherein the transgenic locus comprises
   (iii) a DNA sequence comprising a plurality of human antibody variable region gene segments inserted between endogenous antibody variable region gene segments and an endogenous antibody constant region, whereby endogenous antibody chain expression is inactivated.

The transgenic locus is a heavy chain or light chain locus.

Inactivation of endogenous heavy chain expression in non-human vertebrates such as mice and rats has involved the deletion of all or part of the endogenous heavy chain VDJ region (including sequences between gene segments). The ADAM6 genes are present in the endogenous mouse VDJ region. In mouse, there are two copies of ADAM6 (ADAM6a, ADAM6b) located between the VH and D gene segments in the IgH locus of chromosome 12 (in the intervening region between mouse VH5-1 and D1-1 gene segments). These two adjacent intronless ADAM6 genes have 95% nucleotide sequence identity and 90% amino acid identity. In human and rat, there is only one ADAM6 gene. Expression pattern analysis of mouse ADAM6 shows that it is exclusively expressed in testis [1]. Although ADAM6 transcripts can be detected in lymphocytes, it is restricted to the nucleus, suggesting that the transcription of ADAM6 gene in particular was due to transcriptional read-through from the D region rather than active messenger RNA production [2]. In rat, ADAM6 is on chromosome 6.

Mature ADAM6 protein is located on the acrosome and the posterior regions of sperm head. Notably, ADAM6 forms a complex with ADAM2 and ADAM3, which is required for fertilization in mice [3]. Reference [4] implicates ADAM6 in a model where this protein interacts with ADAM3 after ADAM6 is sulphated by TPST2, sulphation of ADAM6 being critical for stability and/or complex formation involving ADAM6 and ADAM3, and thus ADAM6 and ADAM3 are lost from Tpst2-null sperm. The study observes that Tpst2-deficient mice have male infertility, sperm mobility defects and possible abnormalities in sperm-egg membrane interactions.

Thus, the maintenance of ADAM6 expression in sperm is crucial for fertility. Thus, it is thought that transgenic male mice and rats in which ADAM6 genes have been deleted are not viably fertile. This hampers breeding of colonies and hampers the utility of such mice as transgenic antibody-generating platforms. It would be desirable to provide improved non-human transgenic antibody-generating vertebrates that are fertile.
[1]. Choi l, et. al., Characterization and comparative genomic analysis of intronless Adams with testicular gene expression. Genomics. 2004 April; 83(4):636-46.
[2]. Featherstone K, Wood A L, Bowen A J, Corcoran A E. The mouse immunoglobulin heavy chain V-D intergenic sequence contains insulators that may regulate ordered V(D)J recombination. J Biol Chem. 2010 Mar. 26; 285(13):9327-38. Epub 2010 Jan 25.
[3]. Han C, et. al., Comprehensive analysis of reproductive ADAMs: relationship of ADAM4 and ADAM6 with an ADAM complex required for fertilization in mice. Biol Reprod. 2009 May; 80(5):1001-8. Epub 2009 Jan 7.
[4]. Marcello et al, Lack of tyrosylprotein sulfotransferase-2 activity results in altered sperm-egg interactions and loss of ADAMS and ADAM6 in epididymal sperm, J Biol Chem. 2011 Apr 15:286(15):13060-70, Epub 2011 Feb 21.

According to aspect 109 of the invention, inactivation does not involve deletion of the VDJ region or part thereof including endogenous ADAM6, but instead inactivation by insertion allows for the preservation of endogenous ADAM6 and thus does not risk infertility problems.

The final mouse resulting from the method (or a mouse derived from a cell produced by the method) is in one embodiment a male, so that the invention improves upon the prior art male transgenic mice that are infertile as a result of genomic manipulation. Fertile mice produce sperm that can fertilise eggs from a female mouse. Fertility is readily determined, for example, by successfully breeding to produce an embryo or child mouse. In another embodiment, the method of the invention makes a final female mouse. Such females are, of course, useful for breeding to create male progeny carrying ADAM6 and which are fertile.

In one embodiment of aspect 109, the genome is homozygous for the transgenic locus. For example, the genome is homozygous for endogenous ADAM6 genes.

In one embodiment of the vertebrate of aspect 109, the genome is inactivated for expression of endogenous heavy and kappa (and optionally also lambda) chains.

In one embodiment, in part (iii) of aspect 109 said DNA comprises human VH, D and JH gene segments or human VL and JL gene segments (eg, VK and JK gene segments). In an example, the DNA comprises a landing pad having a selectable marker, eg, a HPRT gene, neomycin resistance gene or a puromycin resistance gene; and/or a promoter.

In one embodiment, in part (iii) of aspect 109 the endogenous gene segments are the entire endogenous VDJ region of a heavy chain locus and/or the endogenous constant region is a Cmu or Cgamma.

In one embodiment, in part (iii) of aspect 109 the endogenous gene segments are the entire endogenous VJ region of a kappa chain locus and/or the endogenous constant region is a Ckappa

In one embodiment, in part (iii) of aspect 109 the endogenous gene segments are the entire endogenous VJ region of a lambda chain locus and/or the endogenous constant region is a Clambda.

The non-human vertebrate cell can be a hybridoma, B-cell, ES cell or an IPS cell. When the cell is an ES cell or IPS cell, the endogenous antibody chain expression is inactivated following differentiation of the cell into a progeny B-cell (eg, in a B-cell in a non-human vertebrate).

The Invention Further Provides:
110. The vertebrate or cell according to aspect 109, wherein said plurality of human antibody gene segments comprises at least 11 human V segments and/or at least 6 human J segments, eg at least 11 human VH gene segments and at least 6 human JH segments and optionally also at least 27 human D segments; optionally with the human inter-gene segment intervening sequences. In an embodiment, the human antibody gene segments are provided by a stretch of DNA sequence of human chromosome 14, comprising the gene segments and intervening sequences in germline configuration.
111. The vertebrate or cell according to aspect 109 or 110, wherein said inserted DNA sequence comprises a human nucleotide sequence comprising said antibody gene segments, wherein the nucleotide sequence is at least 110, 130, 150, 170, 190, 210, 230, 250, 270 or 290 kb. In an embodiment, the nucleotide sequence corresponds to a stretch of DNA sequence of human chromosome 14, comprising the gene segments and intervening sequences in germline configuration, eg, at least a sequence corresponding to the nucleotide sequence from coordinate 106328951 to coordinate 106601551 of a human chromosome 14, eg, a sequence in the GRCH37/hg19 sequence database.
112. The vertebrate or cell according to aspect 109, wherein the transgenic locus is a light chain kappa locus and the human antibody gene segments are between the 3′-most endogenous Jk gene segment and endogenous Ck; optionally wherein the human antibody gene segments comprise five functional human Jλ-Cλ clusters and at least one human VA gene segment, eg, at least a sequence corresponding to the nucleotide sequence from coordinate 23217291 to 23327884 of a lambda locus found on a human chromosome 22.
113. The vertebrate or cell according to any one of aspects 109 to 112, wherein the transgenic locus is a heavy chain locus and the human antibody gene segments are between the 3′-most endogenous JH gene segment (eg, JH4 in a mouse genome) and endogenous Cmu.
114. The vertebrate or cell according to any one of aspects 109 to 113, wherein the genome is homozygous for said transgenic locus.
115. A mouse or mouse cell or a rat or rat cell according to any one of aspects 109 to 114.
116. A method of making a non-human vertebrate cell (optionally a mouse or rat cell), the method comprising
   (a) providing a non-human ES cell whose genome comprises an endogenous antibody chain locus comprising endogenous antibody variable region gene segments and an endogenous antibody constant region; and
   (b) making a transgenic antibody chain locus by inserting into said endogenous locus a DNA sequences comprising a plurality of human antibody variable region gene segments between said endogenous antibody variable region gene segments and said endogenous constant region, so that the human antibody variable region gene segments are operably connected upstream of the endogenous constant region, whereby a non-human vertebrate ES cell is produced that is capable of giving rise to a progeny cell in which endogenous antibody expression is inactivated and wherein the progeny is capable of expressing antibodies comprising human variable regions; and
   (c) optionally differentiating said ES cell into said progeny cell or a non-human vertebrate (eg, mouse or rat) comprising said progeny cell.
117. The method according to aspect 116, wherein said plurality of human antibody gene segments comprises at least 11 human V segments.
118. The method according to aspect 116 or 117, wherein said plurality of human antibody gene segments comprises at least 6 human J segments.
119. The method according to aspect 116, 117 or 118, wherein a human nucleotide sequence is inserted in step (b), the nucleotide sequence comprising said antibody gene segments, wherein the nucleotide sequence is at least 110 kb.
120. The method according to any one of aspects 110 to 113, wherein the endogenous locus is a heavy chain locus and the human antibody gene segments are between the 3′-most endogenous JH gene segment and endogenous Cmu.
121. The method according to any one of aspects 116 to 120, wherein the progeny cell is homozygous for said transgenic locus.

In one embodiment of the method of aspect 116, the method comprises inactivating the genome for expression of endogenous heavy and kappa (and optionally also lambda) chains.

In one embodiment of the method of aspect 116, in part (b) said DNA sequence comprises human VH, D and JH gene segments or human VL and JL gene segments (eg, VK and JK gene segments). In an example, the DNA comprises a landing pad having a selectable marker, eg, a HPRT gene, neomycin resistance gene or a puromycin resistance gene: and/or a promoter.

In one embodiment, in part (b) of aspect 116 the endogenous gene segments are the entire endogenous VDJ region of a heavy chain locus and/or the endogenous constant region is a Cmu or Cgamma.

In one embodiment, in part (b) of aspect 116 the endogenous gene segments are the entire endogenous VJ region of a kappa chain locus and/or the endogenous constant region is a Ckappa

In one embodiment, in part (b) of aspect 116 the endogenous gene segments are the entire endogenous VJ region of a lambda chain locus and/or the endogenous constant region is a Clambda.

The non-human vertebrate cell can be a hybridoma, B-cell, ES cell or an IPS cell. When the cell is an ES cell or IPS cell, the endogenous antibody chain expression is inactivated following differentiation of the cell into a progeny B-cell (eg, in a B-cell in a non-human vertebrate).

The Invention Further Provides:

The method according to aspect 116, wherein said inserted DNA sequence comprises a human nucleotide sequence comprising said human antibody gene segments, wherein the nucleotide sequence is at least 110, 130, 150, 170, 190, 210, 230, 250, 270 or 290 kb. In an embodiment, the nucleotide sequence corresponds to a stretch of DNA sequence of human chromosome 14, comprising the gene segments and intervening sequences in germline configuration, eg, at least a sequence corresponding to the nucleotide sequence from coordinate 106328951 to coordinate 106601551 of a human chromosome 14, eg, a sequence in the GRCH37,/hg19 sequence database.

The method according to aspect 116, wherein the transgenic locus is a light chain kappa locus and the human antibody gene segments are between the 3′-most endogenous Jk gene segment and endogenous Ck; optionally wherein the human antibody gene segments comprise five functional human Jλ-Cλ clusters and at least one human VA gene segment, eg, at least a sequence corresponding to the nucleotide sequence from coordinate 23217291 to 23327884 of a lambda locus found on a human chromosome 22.

The method according to aspect 116, wherein, wherein the transgenic locus is a heavy chain locus and the human antibody gene segments are inserted between the 3′-most endogenous JH gene segment (eg, JH4 in a mouse genome) and endogenous Cmu.
122. The method according to any one of aspects 116 to 121, comprising making the genome of the progeny homozygous for said transgenic locus.

Isolating antibodies from transgenic non-human vertebrates of the invention & useful antigen-specific antibodies of therapeutically-relevant affinities
123. A method of isolating an antibody that binds a predetermined antigen, the method comprising
   (a) providing a vertebrate (optionally a mammal; optionally a mouse or rat according to any one of the above configurations, examples, embodiments or aspects;
   (b) immunising said vertebrate with said antigen (optionally wherein the antigen is an antigen of an infectious disease pathogen);
   (c) removing B lymphocytes from the vertebrate and selecting one or more B lymphocytes expressing antibodies that bind to the antigen;
   (d) optionally immortalising said selected B lymphocytes or progeny thereof, optionally by producing hybridomas therefrom; and
   (e) isolating an antibody (eg, and IgG-type antibody) expressed by the B lymphocytes.
124. The method of aspect 123, comprising the step of isolating from said B lymphocytes nucleic acid encoding said antibody that binds said antigen; optionally exchanging the heavy chain constant region nucleotide sequence of the antibody with a nucleotide sequence encoding a human or humanised heavy chain constant region and optionally affinity maturing the variable region of said antibody; and optionally inserting said nucleic acid into an expression vector and optionally a host.
125. The method of aspect 123 or 124, further comprising making a mutant or derivative of the antibody produced by the method of aspect 122 01123.

As demonstrated by the examples below, the non-human vertebrates of the invention are able to produce antigen-specific antibodies of sub-50 nM affinity with human sequences in their CDR3 regions. Thus, the invention further provides:
126. An antibody or fragment (eg, a Fab or Fab₂) thereof comprising variable regions that specifically bind a predetermined antigen with a sub-50 nM affinity (optionally sub-40, 30, 20, 10, 1, 0.1 or 0.01 nM) as determined by surface plasmon resonance, wherein the antibody is isolated from a non-human vertebrate (optionally a mammal; optionally a mouse or rat) according to any one of the above configurations, examples, embodiments or aspects and comprises heavy chain CDR3s (as defined by Kabat) encoded by a rearranged VDJ of said vertebrate, wherein the VDJ is the product of rearrangement in vivo of a human JH gene segment of a heavy chain locus of said vertebrate with D (optionally a human D gene segment of said locus) and VH gene segments.

In one embodiment, the surface plasmon resonance (SPR) is carried out at 25° C. In another embodiment, the SPR is carried out at 37° C.

In one embodiment, the SPR is carried out at physiological pH, such as about pH7 or at pH7.6 (eg, using Hepes buffered saline at pH7.6 (also referred to as HBS-EP)).

In one embodiment, the SPR is carried out at a physiological salt level, eg, 150 mM NaCl.

In one embodiment, the SPR is carried out at a detergent level of no greater than 0.05% by volume, eg, in the presence of P20 (polysorbate 20; eg, Tween-20™) at 0.05% and EDTA at 3 mM.

In one example, the SPR is carried out at 25° C. or 37° C. in a buffer at pH7.6, 150 mM NaCl, 0.05% detergent (eg, P20) and 3 mM EDTA. The buffer can contain 10 mM Hepes. In one example, the SPR is carried out at 25° C. or 37° C. in HBS-EP. HBS-EP is available from Teknova Inc (California; catalogue number H8022).

In an example, the affinity of the antibody is determined using SPR by
1. Coupling anti-mouse (or other relevant non-human vertebrate) IgG (eg, Biacore BR-1008-38) to a biosensor chip (eg, GLM chip) such as by primary amine coupling;
2. Exposing the anti-mouse IgG (non-human vertebrate antibody) to a test IgG antibody to capture test antibody on the chip;
3. Passing the test antigen over the chip's capture surface at 1024 nM, 256 nM, 64 nM, 16 nM, 4 nM with a 0 nM (i.e. buffer alone); and
4. And determining the affinity of binding of test antibody to test antigen using surface plasmon resonance, eg, under an SPR condition discussed above (eg, at 25° C. in physiological buffer). SPR can be carried out using any standard SPR apparatus, such as by Biacorerm or using the ProteOn XPR36™ (Bio-Rad®).

Regeneration of the capture surface can be carried out with 10 mM glycine at pH1.7. This removes the captured antibody and allows the surface to be used for another interaction. The binding data can be fitted to 1:1 model inherent using standard techniques, eg, using a model inherent to the ProteOn XPR36™ analysis software.

The invention also relates to an scFv, diabody or other antibody fragment comprising a VH and VL domain from an antibody or fragment of aspect 126 (optionally following affinity maturation, eg, by phage display).

In one embodiment, the antigen is a serpin, eg, ovalbumin, antithrombin or antitrypsin. Serpins are a group of proteins with similar structures that were first identified as a set of proteins able to inhibit proteases. The acronym serpin was originally coined because many serpins inhibit chymotrypsin-like serine proteases (serine protease inhibitors). The first members of the serpin superfamily to be extensively studied were the human plasma proteins antithrombin and antitrypsin, which play key roles in controlling blood coagulation and inflammation, respectively. Initially, research focused upon their role in human disease: antithrombin deficiency results in thrombosis and antitrypsin deficiency causes emphysema. In 1980 Hunt and Dayhoff made the surprising discovery that both these molecules share significant amino acid sequence similarity to the major protein in chicken egg white, ovalbumin, and they proposed a new protein superfamily.
127. An antibody or fragment that is identical to an antibody of aspect 126 or a derivative thereof (optionally a derivative whose constant regions are human and/or an affinity matured derivative) that specifically binds said antigen with a sub-50 nM affinity as determined by surface plasmon resonance.
128. A pharmaceutical composition comprising an antibody or fragment of aspect 126 or 127 and a pharmaceutically-acceptable diluent, excipient or carrier.
129. A nucleotide sequence encoding a heavy chain variable region of an antibody or fragment of aspect 126 or 127, optionally as part of a vector (eg, an expression vector).
130. The nucleotide sequence of aspect 129, wherein the sequence is a cDNA derived from a B-cell of the vertebrate from which the antibody of aspect 126 is isolated, or is identical to such a cDNA.
131. An isolated host cell (eg, a hybridoma or a CHO cell or a HEK293 cell) comprising a nucleotide sequence according to aspect 129 or 130.
132. A method of isolating an antibody that binds a predetermined antigen, the method comprising
   (a) providing a vertebrate (optionally a mammal; optionally a mouse or rat according to any one of the above configurations, examples, embodiments or aspects;
   (b) immunising said vertebrate with said antigen;
   (c) removing B lymphocytes from the vertebrate and selecting a B lymphocyte expressing an antibody that binds to the antigen with sub-nM affinity, wherein the antibody is according to aspect 126;
   (d) optionally immortalising said selected B lymphocyte or progeny thereof, optionally by producing hybridomas therefrom; and
   (e) isolating an antibody (eg, and IgG-type antibody) expressed by the B lymphocyte.
133. The method of aspect 132, comprising the step of isolating from said B lymphocyte nucleic acid encoding said antibody that binds said antigen; optionally exchanging the heavy chain constant region nucleotide sequence of the antibody with a nucleotide sequence encoding a human or humanised heavy chain constant region and optionally affinity maturing the variable region of said antibody; and optionally inserting said nucleic acid into an expression vector and optionally a host.
134. The method of aspect 132 or 133, further comprising making a mutant or derivative of the antibody produced by the method of aspect 132 or 133

Inactivation by Inversion of Endogenous VDJ to Qenome Desert Regions
135. A mouse or mouse cell comprising inverted endogenous heavy chain gene segments (eg, VH, D and JH, such as the entire endogenous heavy chain VDJ region) that are immediately 3′ of position 119753123, 119659458 or 120918606 on an endogenous mouse chromosome 12, wherein the mouse comprises a transgenic heavy chain locus comprising a plurality of human VH gene segments, a plurality of human D segments and a plurality of human JH segments operably connected upstream of an endogenous constant region (eg, C mu) so that the mouse or cell (optionally following differentiation into a B-cell) is capable of expressing an antibody comprising a variable region comprising sequences derived from the human gene segments.
136. The mouse or cell of aspect 135, wherein the genome of the mouse or cell is homozygous for said chromosome 12.
137. A cassette for inversion and inactivation of endogenous non-human vertebrate (eg, mouse or rat) antibody chain gene segments, the segments being part of an antibody chain locus sequence on a chromosome of a non-human vertebrate (eg, mouse or rat) cell (eg, ES cell) wherein the sequence is flanked at its 3′ end by a site-specific recombination site (eg, lox, rox or frt), the cassette comprising a nucleotide sequence encoding an expressible label or selectable marker and a compatible site-specific recombination site (eg, lox, rox or frt) flanked by a 5′ and a 3′ homology arm, wherein the homology arms correspond to or are homologous to adjacent stretches of sequence in the cell genome on a different chromosome or on said chromosome at least 10mb away from the endogenous gene segments.
138. A cassette for inversion and inactivation of endogenous mouse antibody heavy chain gene segments, the segments being part of a heavy chain locus sequence on chromosome 12 of a mouse cell (eg, ES cell) wherein the sequence is flanked at its 3′ end by a site-specific recombination site (eg, lox, rox or frt), the cassette comprising a nucleotide sequence encoding an expressible label or selectable marker and a compatible site-specific recombination site (eg, lox, rox or frt) flanked by a 5′ and a 3′ homology arm, wherein (i) the 5′ homology arm is mouse chromosome 12 DNA from coordinate 119753124 to coordinate 119757104 and the 3′ homology arm is mouse chromosome 12 DNA from coordinate 119749288 to 119753123; (ii) the 5′ homology arm is mouse chromosome 12 DNA from coordinate 119659459 to coordinate 119663126 and the 3′ homology arm is mouse chromosome 12 DNA from coordinate 119656536 to 119659458; or (iii) the 5′ homology arm is mouse chromosome 12 DNA from coordinate 120918607 to coordinate 120921930 and the 3′ homology arm is mouse chromosome 12 DNA from coordinate 120915475 to 120918606.
139. A method of inactivating gene segments of an endogenous antibody locus, the method comprising
   (i) Providing a non-human vertebrate cell (eg, an ES cell, eg, a mouse ES cell) whose genome comprises an antibody chain locus comprising endogenous variable region gene segments;
   (ii) Targeting a site-specific recombination site to flank the 3′ of the 3′-most of said endogenous gene segments;
   (iii) Targeting a second site-specific recombination site at least 10mb away from said endogenous gene segments, the second site being compatible with the first site inverted with respect to the first site;
   (iv) Expressing a recombinase compatible with said sites to effect site-specific recombination between said sites, thereby inverting and moving said gene segments away from said locus, wherein the endogenous gene segments are inactivated; and
   (v) Optionally developing the cell into a progeny cell or vertebrate (eg, mouse or rat) whose genome is homozygous for the inversion.
140. A mouse or mouse cell whose genome comprises an inversion of a chromosome 12, wherein the inversion comprises inverted endogenous heavy chain gene segments (eg, VH, D and JH, such as the entire endogenous heavy chain VDJ region); wherein the mouse comprises a transgenic heavy chain locus comprising a plurality of human VH gene segments, a plurality of human D segments and a plurality of human JH segments operably connected upstream of an endogenous constant region (eg, C mu) so that the mouse or cell (optionally following differentiation into a B-cell) is capable of expressing an antibody comprising a variable region comprising sequences derived from the human gene segments; and wherein the inversion is (i) an inversion of mouse chromosome 12 from coordinate 119753123 to coordinate 114666436; (ii) an inversion of mouse chromosome 12 from coordinate 119659458 to coordinate 114666436; or (iii) an inversion of mouse chromosome 12 from coordinate 12091806 to coordinate 114666436.

Other Aspects Include:

A method for producing an antibody specific to a desired antigen the method comprising immunizing a non-human mammal as disclosed herein with the desired antigen and recovering the antibody or a cell producing the antibody.

A method for producing a fully humanised antibody comprising immunizing a non-human mammal as disclosed herein and then replacing the non-human mammal constant region of an antibody specifically reactive with the antigen with a human constant region, suitably by engineering of the nucleic acid encoding the antibody.

A method, cell or mammal as disclosed herein wherein a human coding region DNA sequence is in a functional arrangement with a non-human mammal control sequence, such that transcription of the DNA is controlled by the non-human mammal control sequence. In one aspect the human coding region V, D or J region is in a functional arrangement with a mouse promoter sequence.

The invention also relates to a humanised antibody produced according to any methods disclosed herein and use of a humanised antibody so produced in medicine.

### BRIEF DESCRIPTION OF THE FIGURES

FIGS. 1-8 show an iterative process for insertion of a series of human BACs into a mouse Ig locus

FIG. 3 illustrates targeting of the large insert vector 1 to the mouse ES cell genome. Large insert: Vector 1-engineer the human BAC and target it to the IgH locus. Genotyping: By linearizing the vector at the same position as the insertion of the stuffer sequence it is possible to use a gap-repair genotyping strategy which is short range (1 kb) and highly specific PCR, which could be universal. Validation: 1. CGH (Comparative Genomic Hybridization) tiling array across the BAC sequence. 2. PCR Validation of all critical V, D & J regions.

FIGS. 9-18 show in more detail the process of FIGS. 1-8 for the IgH and kappa locus FIGS. 19 and 20 show the principles behind antibody generation in chimaeric mice FIG. 21 shows a possible insertion site for the human DNA in a mouse chromosome

FIGS. 22-26 disclose an alternative iterative process for insertion of a series of human BACs into a mouse Ig locus

FIGS. 27-29 illustrate a mechanism for inversion of the host VDJ region

FIG. 30 illustrates proof of principle for insertion of a plasmid using an RMCE approach

FIG. 31 illustrates sequential RMCE Integration into Landing Pad

FIG. 32 illustrates confirmation of Successful Insertion into Landing Pad

FIG. 33 illustrates PCR Confirmation of 3′ End Curing

FIG. 34 illustrates insertion of BAC #1 and PCR Diagnostics

FIG. 35 illustrates JH and JK usage

FIG. 36 illustrates DH usage

FIG. 37 illustrates the distribution of CDR-H3 length in human VDJCIJ transcripts from chimera mice

FIG. 38 illustrates the distribution of nucleotide numbers of deletion and insertion in IGH-VDI or IGK-VJ junctions

FIG. 39 illustrates Distribution of JH Usage Within Each VHs

FIG. 40 illustrates Distribution of DH Usage Within Each VHs

FIG. 41A illustrates Nucleotide Gain or Loss at VJ Joints Generates IGK Variants; lines 1, 2-3, 4-7, 8-11, 12-17, 18-19 correspond to SEQ ID NOS: 69, 70, 71, 72, 73, 74, respectively.

FIG. 41B illustrates amino acid sequences of IGK Variants, lines 2-5 (E6, F5, G6), 7-10 (E2), 12-15 (E10, G8), 17-20 (F3, F12), 22-25 (H1) correspond to SEQ ID NOS: 75-79, respectively.

FIG. 42A illustrates Hypermutaion in J Regions Generates IGK Variants; lines 1, 2-11, 12-15, 16-19 correspond to SEQ ID NOS: 80-83, respectively.

FIG. 42B illustrates IGK Variants, lines 2-4, 7-9, 12-14 correspond to SEQ ID NOS: 84-86, respectively.

FIG. 43 illustrates Joint Diversity Produces Functional CDS

FIG. 44 illustrates a plot of identity of JH gene segment use a 5′-RACE Cμ-specific library generated from the splenic B lymphocytes of transgenic mice according to the invention in which endogenous gene segment use has been inactivated by inversion.

FIG. 45 illustrates the ratio of mouse VH to human VH usage as determined from antibody sequences from splenic B lymphocytes of transgenic mice according to the invention in which endogenous gene segment use has been inactivated by inversion.

FIG. 46 illustrates inversion strategy schematic

FIG. 47 illustrates targeting construct R57 for inversion

FIG. 48 illustrates sequence analysis from a Cμ-specific 5′-RACE library of splenic B lymphocytes of S1^{inv1} (one human IGH BAC (ie, multiple human VH, all functional human D and JH) with an inverted endogenous IGH locus) mouse shows that practically all the transcripts came from rearranged human VH-D-JH gene segments

FIG. 49 illustrates that the S1^{inv1} mouse shows a similar usage of both D and JH gene segments to human

FIG. 50 illustrates that mouse VH usage is further significantly reduced following insertion of the 2 nd human BAC into the endogenous heavy chain locus

FIG. 51 illustrates a gel showing that normal class-switching (to IgG-type) was observed in transcripts from mice of the invention. The rearranged transcripts were detected using RT-PCR with human VH-specific and mouse Cγ-specific primers for amplification from peripheral blood cells of immunized transgenic mice

FIG. 52 illustrates sequence analysis amplified fragments demonstrate hypermutation occurred within the human variable regions of these IGγ chains from mice of the invention

FIG. 53 illustrates Flow cytometric analysis showing normal B-cell compartments in transgenic mice of the invention

FIGS. 54A-54D illustrate normal IgH isotypes in transgenic mice (H1) immunised with 100 pg Cholera Toxin B subunit. FIGS. 54E-54H illustrate normal IgH isotypes in transgenic mice (S1) immunised with 100 pg Cholera Toxin B subunit.

FIG. 55A and FIG. 55B illustrate normal IgH isotypes and serum levels are obtained in transgenic I-11 and S1 animals, respectively, of the invention following immunisation with antigens.

Sequences

SEQ ID No 1 is a Rat switch sequence
SEQ ID No 2 is a landing pad targeting vector (long version)
SEQ ID No 3 is a landing pad targeting vector (shorter version)
SEQ ID No 4 is the mouse strain 129 switch
SEQ ID No 5 is the mouse strain C57 switch
SEQ ID No 6 is the 5′ homology arm of a landing pad
SEQ ID No 7 is oligo HV2-5
SEQ ID No 8 is oligo HV4-4
SEQ ID No 9 is oligo HV1-3
SEQ ID No 10 is oligo HV1-2
SEQ ID No 11 is oligo HV6-1
SEQ ID No 12 is oligo Cμ
SEQ ID No 13 is oligo KV1-9
SEQ ID No 14 is oligo KV1-8
SEQ ID No 15 is oligo KV1-6
SEQ ID No 16 is oligo KV1-5
SEQ ID No 17 is oligo Cκ
SEQ ID Nos 18-20 are rat switch sequences
SEQ ID No 21 is X₁X₂ T F G Q, where X₁X₂ = PR, RT, or PW
SEQ ID No 22 is X₁X₂ T F G Q G T K V E I K R A D A, where X₁X₂ = PR, RT, or PW;
SEQ ID No 23 is X₃X₄ T F G Q, where X₃X₄ = PR or PW
SEQ ID No 24 is X₃X₄ T F G Q G T K V E I K R A D A, where X₃X₄ = PR or PW
SEQ ID No 25 is Primer E1554
SEQ ID No 26 is Primer E1555
SEQ ID No 27 is Primer ELP1352_Cγ1
SEQ ID No 28 is Primer ELP1353_Cγ2b
SEQ ID No 29 is Primer ELP1354_Cγ2a
SEQ ID No 30 is Primer ELP1356_VH4-4
SEQ ID No 31 is Primer ELP1357_VH1-2,3
SEQ ID No 32 is Primer ELP1358_VH6-1
SEQ ID No 33 is Primer mIgG1_2 rev
SEQ ID No 34 is Primer mIgG2b rev
SEQ ID No 35 is Primer mIgG2a_2 rev
SEQ ID No 36 is Primer mCH1 unirev
SEQ ID No 37 is Primer mCH1 unirev_2
SEQ ID Nos 38-45 are CDRH3 sequences
SEQ ID Nos 46-50 is 3, 4, 5, 6 or more (up to 82) repeats of GGGCT
SEQ ID NOs 51-55 are heavy chain CDR1 sequences against CTB (cloned and reference)
SEQ ID NOs 56-60 are heavy chain CDR2 sequences against CTB (cloned and reference)
SEQ ID NOs 61-63 are heavy chain CDR3 sequences against CTB (cloned and reference)
SEQ ID NOs 64-68 are J Region sequences against CTB (cloned and reference)

### DETAILED DESCRIPTION OF THE INVENTION

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As a source of antibody gene segment sequences, the skilled person will also be aware of the following available databases and resources (including updates thereof) the contents of which are incorporated herein by reference:

The Kabat Database (G. Johnson and T. T. Wu, 2002; World Wide Web (www) kabatdatabase.com). Created by E. A. Kabat and T. T. Wu in 1966, the Kabat database publishes aligned sequences of antibodies, T-cell receptors, major histocompatibility complex (MHC) class I and II molecules, and other proteins of immunological interest. A searchable interface is provided by the SeqhuntII tool, and a range of utilities is available for sequence alignment, sequence subgroup classification, and the generation of variability plots. See also Kabat, E. A., Wu, T. T., Perry, H., Gottesman, K., and Foeller, C. (1991) Sequences of Proteins of Immunological Interest, 5th ed., NIH Publication No. 91-3242, Bethesda, MD, which is incorporated herein by reference, in particular with reference to human gene segments for use in the present invention.

KabatMan (A. C. R. Martin, 2002; World Wide Web (www) bioinf.org.uk/abs/simkab.html). This is a web interface to make simple queries to the Kabat sequence database.

IMGT (the International ImMunoGeneTics Information System; M.-P. Lefranc, 2002; World Wide Web (www) imgt.cines.fr). IMGT is an integrated information system that specializes in antibodies, T cell receptors, and MHC molecules of all vertebrate species. It provides a common portal to standardized data that include nucleotide and protein sequences, oligonucleotide primers, gene maps, genetic polymorphisms, specificities, and two-dimensional (2D) and three-dimensional (3D) structures. IMGT includes three sequence databases (IMGT/LIGM-DB, IMGT/MHC-DB, IMGT/PRIMERDB), one genome database (11 1./IGT/GENE-DB), one 3D structure database (IMGT/3Dstructure-DB), and a range of web resources (“IMGT Marie-Paule page”) and interactive tools.

V-BASE (I. M. Tomlinson, 2002; World Wide Web (www) mrc-cpe.cam.ac.ukivbase). V-BASE is a comprehensive directory of all human antibody germline variable region sequences compiled from more than one thousand published sequences. It includes a version of the alignment software DNAPLOT (developed by Hans-Helmar Althaus and Werner Muller) that allows the assignment of rearranged antibody V genes to their closest germline gene segments.

Antibodies—Structure and Sequence (A. C. R. Martin, 2002; World Wide Web (www) bioinforg.ukIabs). This page summarizes useful information on antibody structure and sequence. It provides a query interface to the Kabat antibody sequence data, general information on antibodies, crystal structures, and links to other antibody-related information. It also distributes an automated summary of all antibody structures deposited in the Protein Databank (PDB). Of particular interest is a thorough description and comparison of the various numbering schemes for antibody variable regions.

AAAAA (A Ho's Amazing Atlas of Antibody Anatomy; A. Honegger, 2001; World Wide Web (www) unizh.cht—antibody). This resource includes tools for structural analysis, modeling, and engineering. It adopts a unifying scheme for comprehensive structural alignment of antibody and T-cell-receptor sequences, and includes Excel macros for antibody analysis and graphical representation.

WAM (Web Antibody Modeling; N. Whitelegg and A. R. Rees, 2001; World Wide Web (www) antibody.bath.ac.uk). Hosted by the Centre for Protein Analysis and Design at the University of Bath, United Kingdom. Based on the AbM package (formerly marketed by Oxford Molecular) to construct 3D models of antibody Fv sequences using a combination of established theoretical methods, this site also includes the latest antibody structural information.

Mike's Immunoglobulin Structure/Function Page (M, R. Clark, 2001; World Wide Web (www) path.cam.ac.uk/—inrc7hnikeirnages,html) These pages provide educational materials on immunoglobulin structure and function, and are illustrated by many colour images, models, and animations. Additional information is available on antibody humanization and Mike Clark's Therapeutic Antibody Human Homology Project, which aims to correlate clinical efficacy and anti-immunoglobulin responses with variable region sequences of therapeutic antibodies.

The Antibody Resource Page (The Antibody Resource Page, 2000; World Wide Web (www) antibodyresource.com). This site describes itself as the “complete guide to antibody research and suppliers.” Links to amino acid sequencing tools, nucleotide antibody sequencing tools, and hybridoma/cell-culture databases are provided.

Humanization by Design (J. Sa Id a n ha 2000; World Wide Web (www) people.cryst.bbk.ac.uk/—ubcg07s). This resource provides an overview on antibody humanization technology. The most useful feature is a searchable database (by sequence and text) of more than 40 published humanized antibodies including information on design issues, framework choice, framework back-mutations, and binding affinity of the humanized constructs.

See also Antibody Engineering Methods and Protocols, Ed. Benny K C Lo, Methods in Molecular Biology™, Human Press. Also at World Wide Web (wwvv) blogsua.com/pdf/antibody-engineering-methods-and-protocolsantibody-engineering-methods-and-protocols,pdf

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The following examples are put forth so as to provide those of ordinary skill in the art with a complete disclosure and description of how to make and use the invention, and are not intended to limit the scope of what the inventors regard as their invention.

### EXAMPLES

### Example 1

BAC Recombineering

Overall strategy: A mouse model of the invention can be achieved by inserting ˜960 kb of the human heavy chain locus containing all the V, D and J-regions upstream of the mouse constant region and 473 kb of the human kappa region upstream of the mouse constant region. Alternatively, or in tandem, the human lambda region is inserted upstream of the mouse constant region. This insertion is achieved by gene targeting in ES cells using techniques well known in the art.

High fidelity insertion of intact V-D-J regions into each locus in their native (wild-type) configuration is suitably achieved by insertion of human bacterial artificial chromosomes (BACs) into the locus. Suitably the BACs are trimmed so that in the final locus no sequence is duplicated or lost compared to the original. Such trimming can be carried out by recombineering.

The relevant human BACs, suitably trimmed covering these loci are on average 90 kb in size.

In one approach the full complement of human D and J-elements as well as seven or eight human V-regions are covered by the first BACs to be inserted in the experimental insertion scheme described below. The first BACs to be inserted in the IgH and IgK loci may contain the following V-regions. IgH:V6-1, VII-1-1, V1-2, VIII-2-1, V1-3, V4-4, V2-5 and IgK: V4-1, V5-2, V7-3, V2-4, V1-5, V1-6, V3-7, V1-8.

Suitably the performance of each locus is assessed after the first BAC insertion using chimeric mice and also after each subsequent BAC addition. See below for detailed description of this performance test.

Nine additional BAC insertions will be required for the IgH locus and five for IgK to provide the full complement of human V-regions covering all 0.96 Mb and 0.473 Mb of the IgH and OK loci, respectively.

Not all BACs retain their wild-type configuration when inserted into the ES cell genome. Thus, high density genomic arrays were deployed to screen ES cells to identify those with intact BAC insertions (Barrett, M. T., Scheffer, A., Ben-Dor, A., Sampas, N., Lipson, D., Kincaid, R., Tsang, P., Curry, B., Baird, K., Meltzer, P. S., et al. (2004). Comparative genomic hybridization using oligonucleotide microarrays and total genomic DNA. Proceedings of the National Academy of Sciences of the United States of America 101, 17765-17770.).This screen also enables one to identify and select against ES clones in which the ES cell genome is compromised and thus not able to populate the germ line of chimeric animals. Other suitable genomic tools to facilitate this assessment include sequencing and PCR verification.

Thus in one aspect the correct BAC structure is confirmed before moving to the next step.

It is implicit from the description above that in order to completely engineer the loci with 90 kb BACs, it is necessary to perform a minimum of 10 targeting steps for IgH and 5 steps for the IgK. Mice with an IgL locus can be generated in a similar manner to the IgK locus. Additional steps are required to remove the selection markers required to support gene targeting. Since these manipulations are being performed in ES cells in a step-wise manner, in one aspect germ line transmission capacity is retained throughout this process.

Maintaining the performance of the ES cell clones through multiple rounds of manipulation without the need to test the germ line potential of the ES cell line at every step may be important in the present invention. The cell lines currently in use for the KOMP and EUCOMM global knockout projects have been modified twice prior to their use for this project and their germ line transmission rates are unchanged from the parental cells (these lines are publicly available, see World Wide Web (www) komp.org and World Wide Web (www) eucomm.org). This cell line, called JR/18, can generate 100% ES cell-derived mice under published culture conditions (Pettitt, S. J., Liang, Q., Rairdan, X. Y., Moran, J. L., Prosser, H. M., Beier, D. R., Lloyd, K. C., Bradley, A., and Skarnes, W. C. (2009). Agouti C57BLi6N embryonic stem cells for mouse genetic resources. Nature Methods.). These cells have demonstrated ability to reproducibly contribute to somatic and germ line tissue of chimaeric animals using standard mouse ES cell culture conditions. This capability can be found with cells cultured on a standard feeder cell line (SNL) and even feeder-free, grown only on gelatine-coated tissue culture plates. One particular sub-line, JM8A3, maintained the ability to populate the germ line of chimeras after several serial rounds of sub-cloning. Extensive genetic manipulation via, for example; homologous recombination — as would be the case in the present invention — cannot compromise the pluripotency of the cells. The ability to generate chimeras with such high percentage of ES cell-derived tissue has other advantages. First, high levels of chimerism correlates with germ line transmission potential and provide a surrogate assay for germ line transmission while only taking 5 to 6 weeks. Second, since these mice are 100% ES cell derived the engineered loci can be directly tested, removing the delay caused by breeding. Testing the integrity of the new Ig loci is possible in the chimera since the host embryo will be derived from animals that are mutant for the RAG-1 gene as described in the next section.

Another cell line that may be used is an HPRT-ve cell line, such as AB2.1, as disclosed in Ramirez-Solis R, Liu P and Bradley A, “Chromosome engineering in mice,” Nature, 1995;378;6558;720-4.

RAG-1 complementation: While many clones will generate 100% ES derived mice some will not. Thus, at every step mice are generated in a RAG-1-deficient background. This provides mice with 100% ES-derived B- and T-cells which can be used directly for immunization and antibody production. Cells having a RAG-2 deficient background, or a combined RAG-1/RAG-2 deficient background may be used, or equivalent mutations in which mice produce only ES cell-derived B cells and/or T cells.

In order that only the human-mouse IgH or IgK loci are active in these mice, the human-mouse IgH and OK loci can be engineered in a cell line in which one allele of the IgH or IgK locus has already been inactivated. Alternatively the inactivation of the host Ig locus, such as the IgH or IgK locus, can be carried out after insertion.

Mouse strains that have the RAG-1 gene mutated are immunodeficient as they have no mature B-or T-lymphocytes (U.S. Pat. No. 5,859,307). T- and B-lymphocytes only differentiate if proper V(D)J recombination occurs. Since RAG-1 is an enzyme that is crucial for this recombination, mice lacking RAG-1 are immunodeficient. If host embryos are genetically RAG-1 homozygous mutant, a chimera produced by injecting such an embryo will not be able to produce antibodies if the animal's lymphoid tissues are derived from the host embryo. However, JM8 cells and AB2,1 cells, for example, generally contribute in excess of 80% of the somatic tissues of the chimeric animal and would therefore usually populate the lymphoid tissue. JM8 cells have wild-type RAG-1 activity and therefore antibodies produced in the chimeric animal would be encoded by the engineered JM8 ES cell genome only. Therefore, the chimeric animal can be challenged with an antigen by immunization and subsequently produce antibodies to that antigen. This allows one skilled in the art to test the performance of the engineered human/mouse IgH and IgK loci as described in the present invention. See FIGS. 19 and 20.

One skilled in the art would use the chimeric animal as described to determine the extent of antibody diversity (see e.g. Harlow, E. & Lane, D. 1998, 5^{th} edition, Antibodies: A Laboratory Manual, Cold Spring Harbor Lab. Press, Plainview, NY). For example, the existence in the chimeric animal's serum of certain antibody epitopes could be ascertained by binding to specific anti-idiotype antiserum, for example, in an ELISA assay. One skilled in the art could also sequence the genomes of B-cell clones derived from the chimeric animal and compare said sequence to wild-type sequence to ascertain the level of hypermutation, such hypermutation indicative of normal antibody maturation.

One skilled in the art would also use said chimeric animal to examine antibody function wherein said antibodies are encoded from the engineered Ig loci (see e.g. Harlow, E. & Lane, D. 1998, 5^{th} edition, Antibodies: A Laboratory Manual, Cold Spring Harbor Lab. Press, Plainview, NY). For example, antisera could be tested for binding an antigen, said antigen used to immunize the chimeric animal. Such a measurement could be made by an ELISA assay. Alternatively, one skilled in the art could test for neutralization of the antigen by addition of the antisera collected from the appropriately immunized chimeric animal.

It is well known to those skilled in the art that positive outcomes for any of these tests demonstrate the ability of the engineered Ig loci, the subject of the instant invention, to encode antibodies with human variable regions and mouse constant regions, said antibodies capable of functioning in the manner of wild-type antibodies.

Experimental Techniques: Recombineering for the production of vectors for use in homologous recombination in ES cells is disclosed in, for example, WO9929837 and WO0104288, and the techniques are well known in the art. In one aspect the recombineering of the human DNA takes place using BACs as a source of said human DNA. Human BAC DNA will be isolated using QIAGEN®, BAC purification kit. The backbone of each human BAC will be modified using recombineering to the exact same or similar configuration as the BAC already inserted into the mouse IgH region. The genomic insert of each human BAC will be trimmed using recombineering so that once the BACs are inserted, a seamless contiguous part of the human V(D)J genomic region will form at the mouse IgH or IgK locus. BAC DNA transfection by electroporation and genotyping will be performed accordingly to standard protocols (Prosser, H. M., Rzadzinska, A. K., Steel, K. P., and Bradley, A. (2008). “Mosaic complementation demonstrates a regulatory role for myosin Vlla in actin dynamics of stereocilia.” Molecular and Cellular Biology 28, 1702-1712; Ramirez-Solis, R., Davis, A. C., and Bradley, A. (1993). “Gene targeting in embryonic stem cells.” Methods in Enzymology 225, 855-878.). Recombineering will be performed using the procedures and reagents developed by Pentao Liu and Don Court's laboratories (Chan, W., Costantino, N., Li, R., Lee, S.C.. Su. Q., Melvin, D., Court, D. L., and Liu, P. (2007). “A recombineering based approach for high-throughput conditional knockout targeting vector construction.” Nucleic Acids Research 35, e64).

These and other techniques for gene targeting and recombination of BAC-derived chromosomal fragments into a non-human mammal genome, such as a mouse are well-known in the art and are disclosed in, for example, in World Wide Web (www) eucomm.org/information/targeting and World Wide Web (www) eucomm.orglinformation/publications.

Cell culture of C57BLI6N-derived cell lines, such as the JM8 male ES cells will follow standard techniques. The JM8 ES cells have been shown to be competent in extensively contributing to somatic tissues and to the germline, and are being used for large mouse mutagenesis programs at the Sanger Institute such as EUCOMM and KOMP (Pettitt, S. J., Liang, Q., Rairdan, X,Y, Moran, J. L., Prosser, H. M., Beier, D. R, Lloyd, K. C., Bradley, A., and Skarnes, W. C. (2009). “Agouti C57BL/6N embryonic stem cells for mouse genetic resources.” Nature Methods.). JM8 ES cells (1.0×10⁷) will be electroporated (500 pF, 230V; Bio-Rad®) with 10 μg/ml linearized human BAC DNA. The transfectants will be selected with either Puromycin (3 μg/ml) or G418 (150 μg/ml). The selection will begin either 24 hours (with G418) or 48 hours (with Puromycin) post electroporation and proceed for 5 days. 10 μg linearized human BAC DNA can yield up to 500 Puromycin or G418 resistant ES cell colonies. The antibiotic resistant ES cell colonies will be picked into 96-well cell culture plates for genotyping to identify the targeted clones.

Once targeted mouse ES cell clones are identified, they will be analyzed by array Comparative Genomic Hybridization (CGH) for total genome integrity (Chung, Y. J., Jonkers, J., Kitson, H.. Fiegler, H., Humphray, S., Scott, C., Hunt, S., Yu, Y., Nishijima, I., Velds, A., et al. (2004). “A whole-genome mouse BAC microarray with 1-Mb resolution for analysis of DNA copy number changes by array comparative genomic hybridization.” Genome research 14, 188-196. and Liang, Q., Conte, N., Skarnes, W. C., and Bradley, A. (2008). “Extensive genomic copy number variation in embryonic stem cells.” Proceedings of the National Academy of Sciences of the United States of America 105, 17453-17456.). ES cells that have abnormal genomes do not contribute to the germline of the chimeric mice efficiently. BAC integrity will be examined by PCR-amplifying each known functional V gene in the BAC. For example, in one approach the first human BAC chosen for the IgH locus has 6 functional V genes. To confirm the integrity of this BAC for the presence of these 6 IGH V genes, at least 14 pairs of PCR primers will be designed and used to PCR-amplify genomic DNA from the targeted ES cells. The human wild-type size and sequence of these fragments will ensure that the inserted BAC has not been rearranged.

More detailed CGH will also confirm the integrity of the inserted BACs. For example, one skilled in the art could use an oligo aCGH platform, which is developed by Agilent Technologies, Inc. This platform not only enables one to study genome-wide DNA copy number variation at high resolution (Barrett, M. T., Scheffer, A., Ben-Dor, A., Sampas, N., Lipson, D., Kincaid, R., Tsang, P., Curry, B., Baird, K., Meltzer, P. S., et al. (2004). “Comparative genomic hybridization using oligonucleotide microarrays and total genomic DNA.” Proceedings of the National Academy of Sciences of the United States of America 101, 17765-17770.), but permit examination of a specific genome region using custom designed arrays. Comparing the traditional aCGH techniques which rely on cDNA probes or whole BAC probes, the 60-mer oligonucleotides probes can ensure specific hybridization and high sensitivity and precision that is needed in order to detect the engineered chromosome alterations that were made. For example, oligos designed to hybridize at regular intervals along the entire length of the inserted BAC would detect even quite short deletions, insertions or other rearrangements. Also, this platform provides the greatest flexibility for customized microarray designs. The targeted ES cell genomic DNA and normal human individual genomic DNA will be labelled separately with dyes and hybridized to the array. Arrays slides will be scanned using an Aglient Technologies DNA microarray scanner. Reciprocal fluorescence intensities of dye Cy5 and dye Cy3 on each array image and the log 2 ratio values will be extracted by using Bluefuse software (Bluegnome). Spots with inconsistent fluorescence patterns (“confidence”<0.29 or “quality”=0) will be excluded before normalizing all log 2 ratio values. Within an experiment, Log2 ratio between −0.29 and +0.29 for the signal from any oligo probe are regarded as no copy number change. The log 2 ratio threshold for “Duplication” is usually >0.29999, and for deletion is <0.29999.

Once the first human BAC is inserted into the mouse IgH locus and confirmed to be in its intact, native configuration, the FRT-flanked BAC backbone will be excised by using Flp site-specific recombinase. If regular Flp-catalyzed FRT recombination is not high enough, one can use Flo, an improved version of Flpo recombinase which in certain tests is 3-4 times more efficient than the original Flp in ES cells. After the BAC backbone is excised, ES cells will become sensitive to Puromycin (or G418) and resistant to FIAU (for loss of the TK cassette). The excision events will be further characterized by PCR amplification of the junction fragment using human genomic DNA primers. These FRT-flankecl BAC backbone-free ES cells will be used for the next round of human BAC insertion and for blastocyst injection.

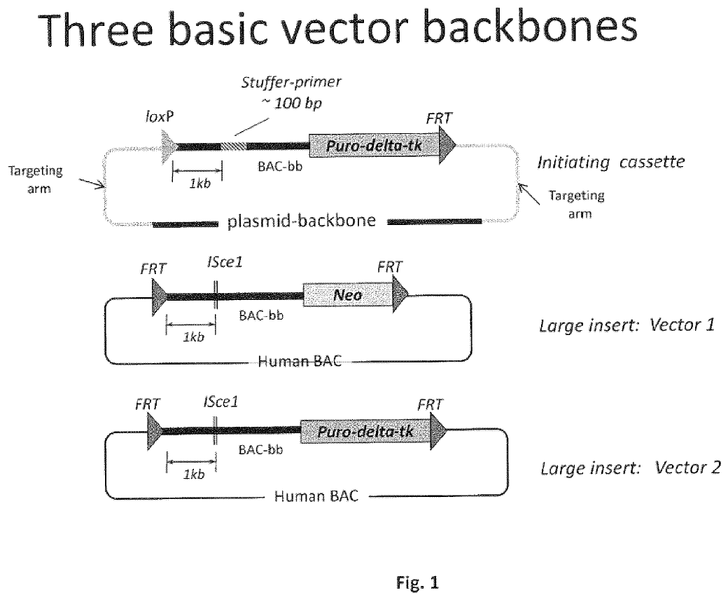

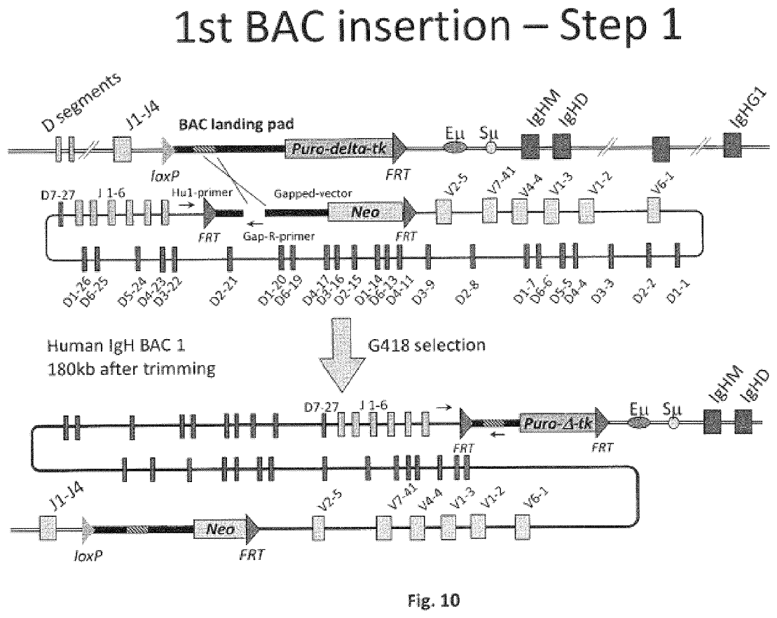

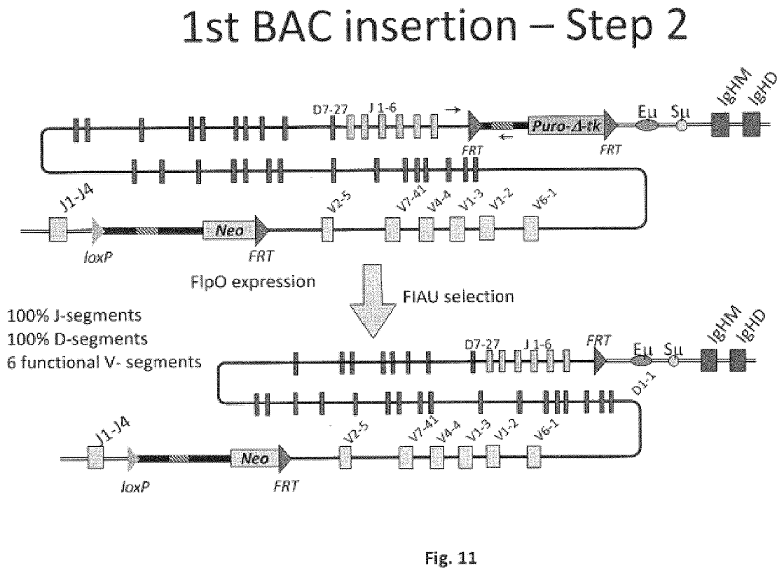

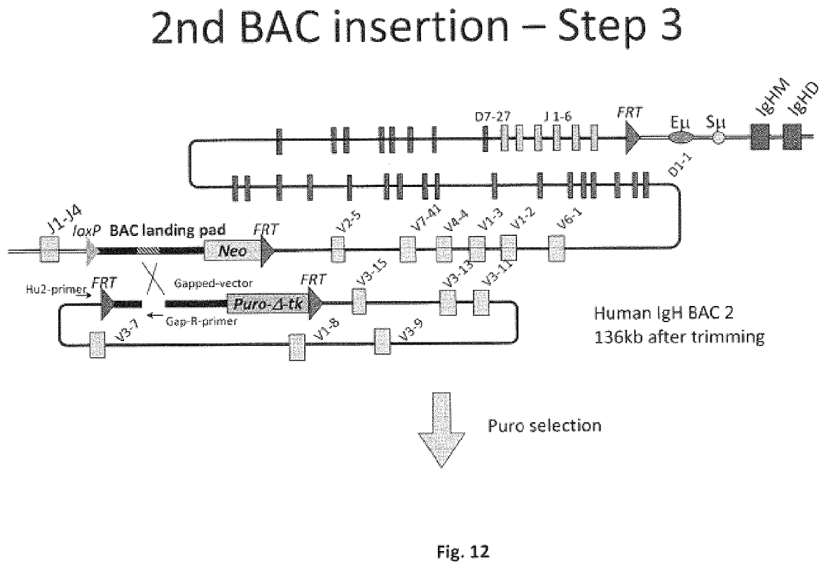

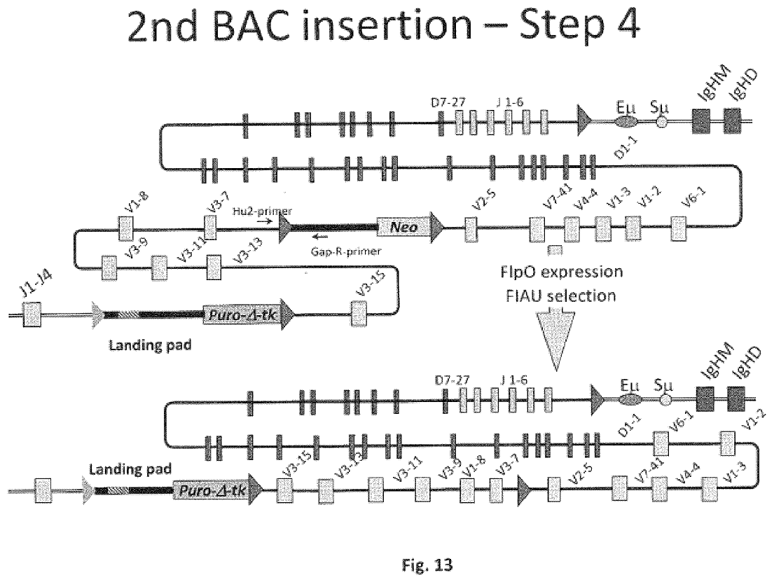

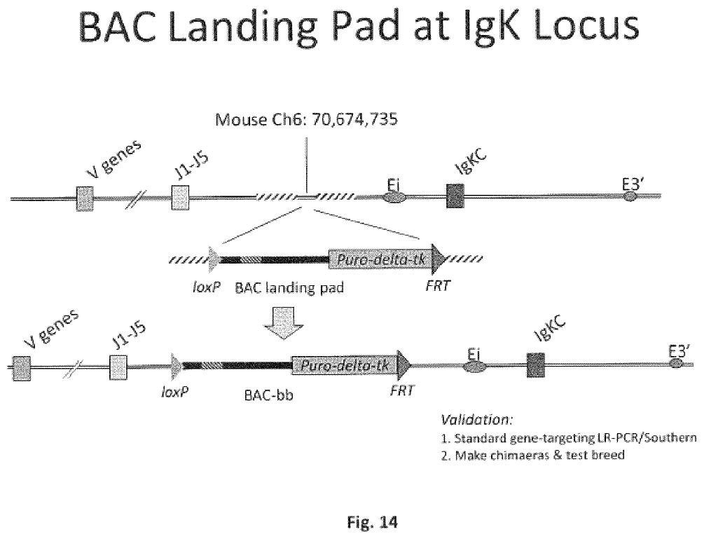

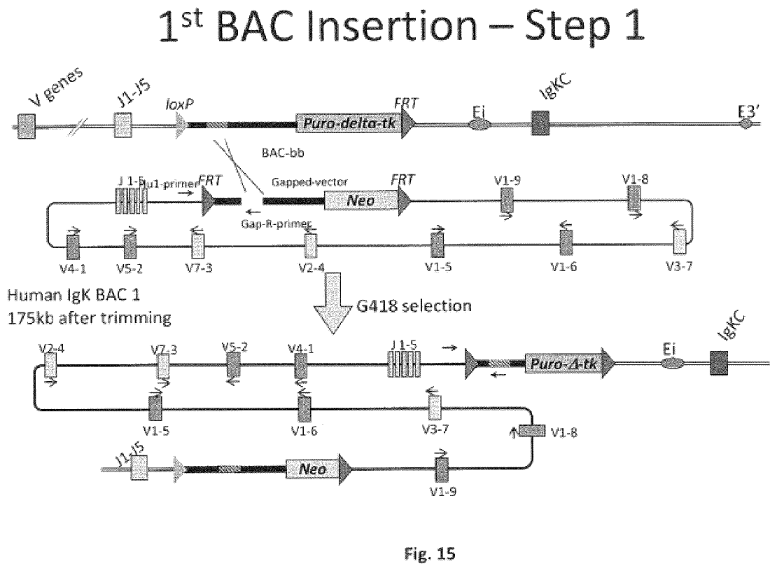

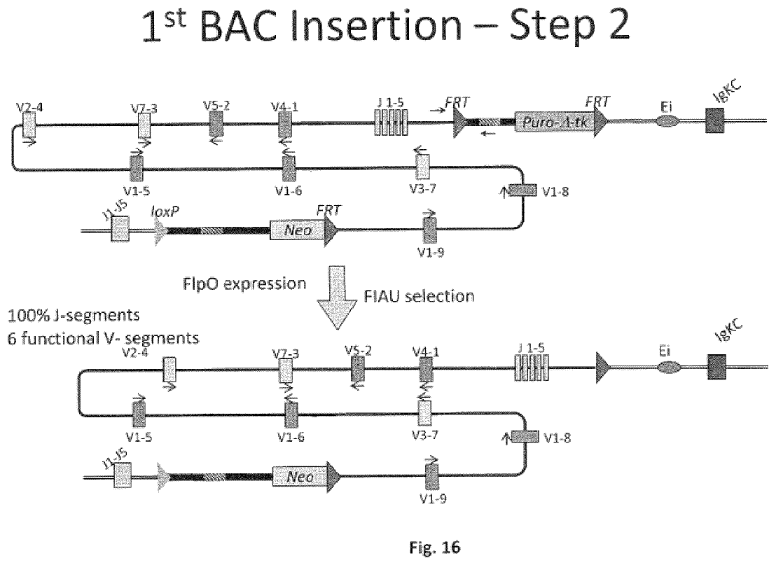

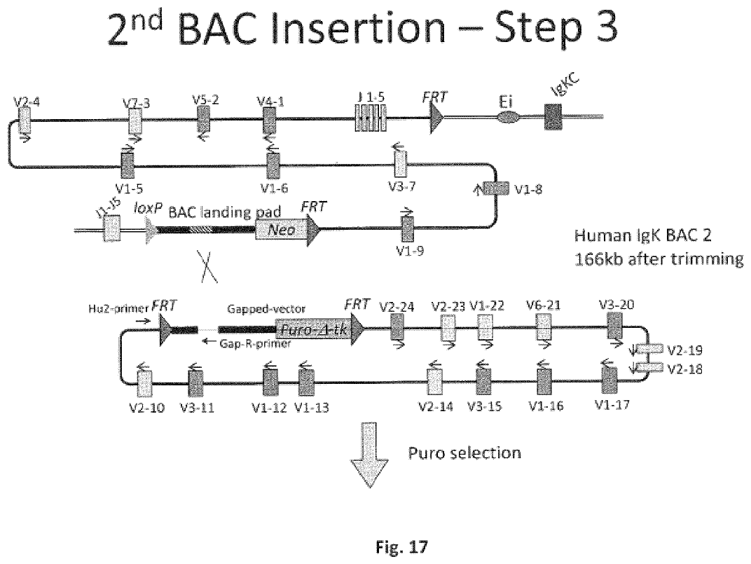

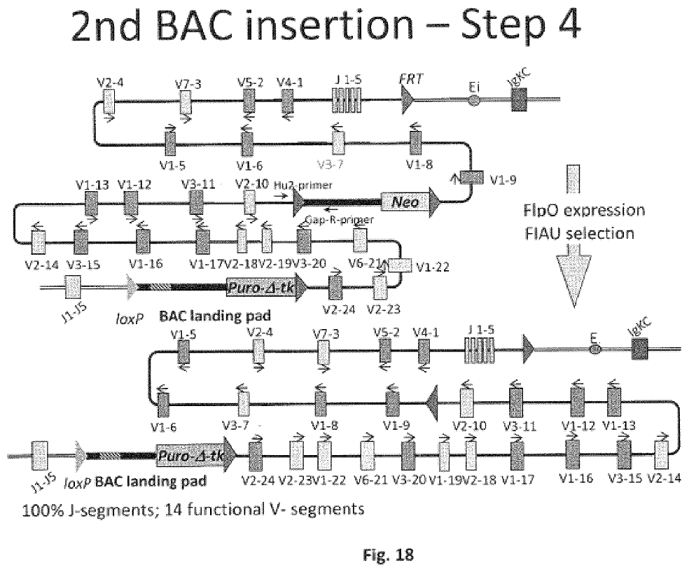

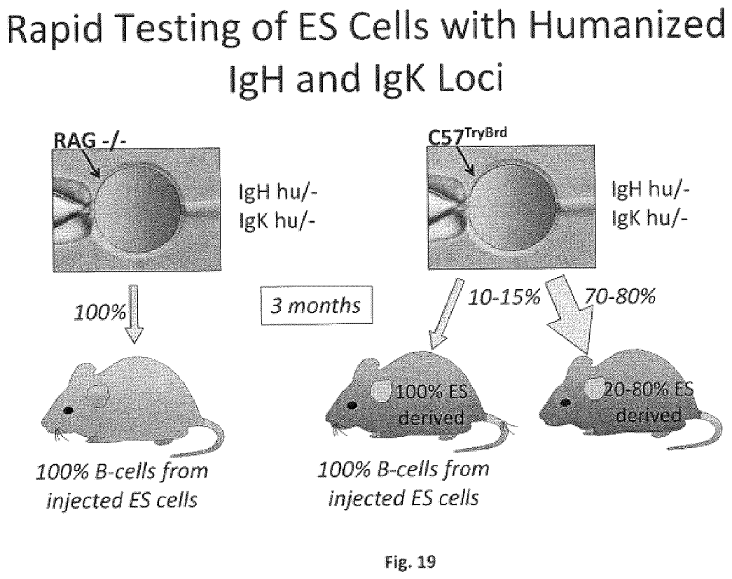

Targeting of the genome of an ES cell to produce a transgenic mouse may be carried out using a protocol as explained by reference to the attached FIGS. 1-18.

FIG. 1 illustrates three basic backbone vectors; an initiating cassette and 2 large insert vectors 1 and 2 respectively. The initiating cassette comprises sequences homologous to the desired site of insertion into the mouse genome, those sites flanking a selectable marker and stuffer primer sequence for PCR based genotyping to confirm correct insertion of BACs. The Stuffer-primer sequence provides the basis for genotyping each BAC addition step. This sequence is considered to provide a robust well validated sequence template for PCR primer and may be located at the IScel site, ideally ˜1 kb from the BAC insert.

The large insert vectors comprise human DNA on plasmids with selectable markers and a unique restriction site for linearisation of the plasmid to aid in homologous recombination into the genome of the ES cell.

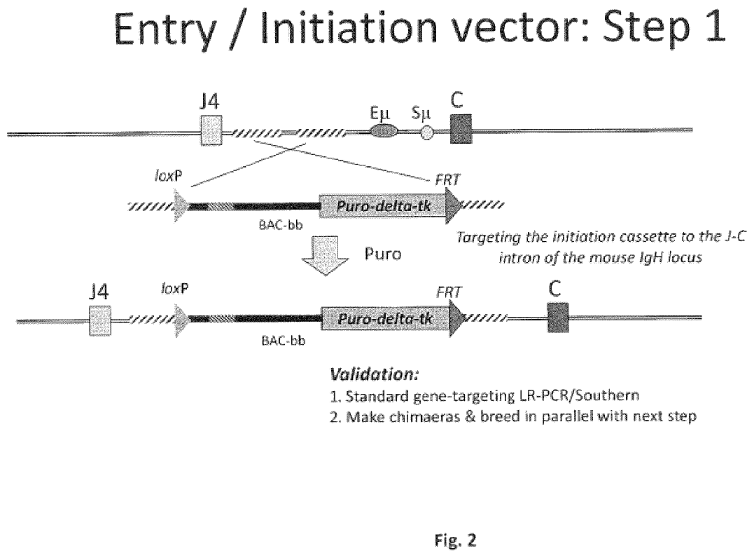

FIG. 2 illustrates insertion of an initiating cassette into the mouse genome by Homologous recombination between the mouse J4 and C alpha exons. Puromycin selection allows identification of ES cells with insertion of the cassette. pu(Delta)tk is a bifunctional fusion protein between puromycin N-acetyltransferase (Pura) and a truncated version of herpes simplex virus type 1 thymidine kinase (DeltaTk). Murine embryonic stem (ES) cells transfected with pu(Delta)tk become resistant to puromycin and sensitive to 1+2-deoxy-2-fluoro-1-beta-D-arabina-furanosyl)-5-iodouracil (FIAU). Unlike other HSV1 tk transgenes, puDeltatk is readily transmitted through the male germ line. Thus pu(Delta)tk is a convenient positive/negative selectable marker that can be widely used in many ES cell applications.

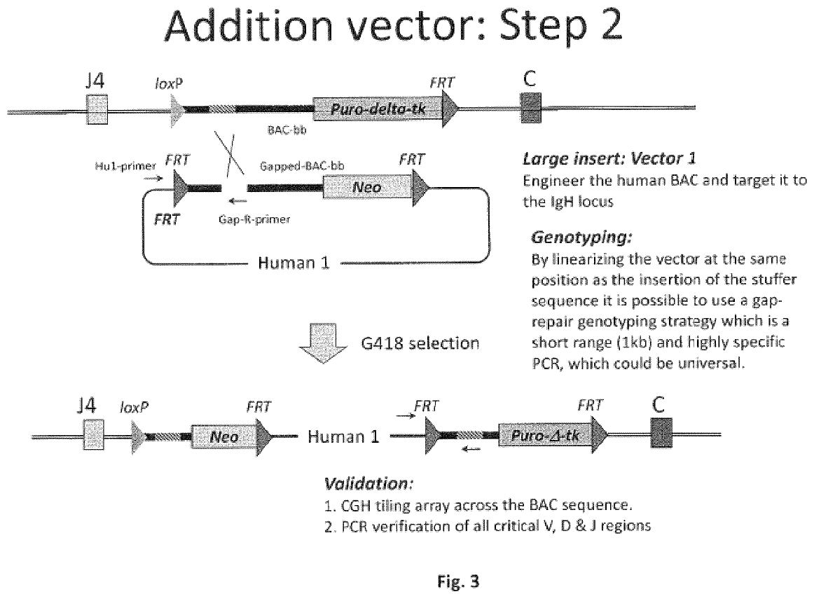

FIG. 3 illustrates targeting of the large insert vector 1 to the mouse ES cell genome. Linearisation of the vector is made at the same position as the stuffer primer sequence which allows for a gap repair genotyping strategy, well known in the art—see Zheng et al NAR 1999. Vol 27. 11, 2354-2360. In essence, random insertion of the targeting vector into the genome will not ‘repair’ the gap whereas a homologous recombination event will repair the gap. Juxtaposition of appropriate PCR primer sequences allows colonies to be screened individually for a positive PCR fragment indicating proper insertion. Positive selection using G418 allows for identification of mouse ES cells containing the neo selection marker. PCR verification can be made of all critical V, D and J regions. Array comparative genomic hybridization can be used to validate the BAC structure.

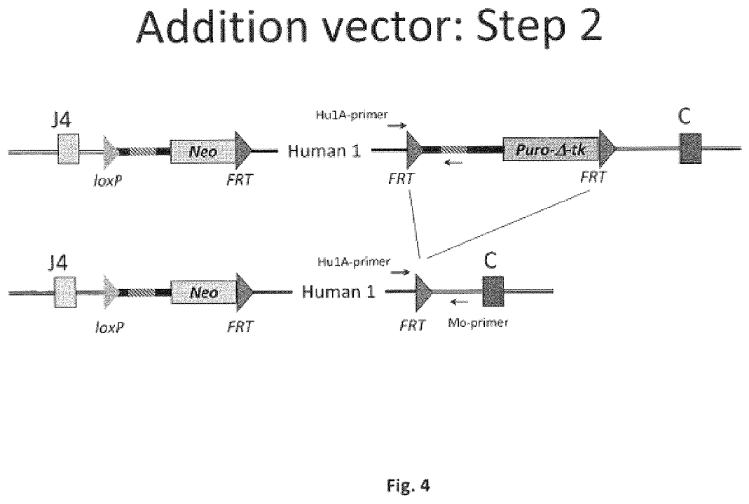

FIG. 4 illustrates the euro-delta-tk cassette and the BAC plasmid backbone is deleted using Flpe and select in FIAU. Since Flpe works inefficiently in mouse ES cells (5% deletion with transient Flpe expression), it is expected that in most cases, the recombination occurs between the two FRT sites flanking the BAC backbone. Flpo can also be tested to find out the recombination efficiency between two FRT sites that are 10 kb away.

Given that the FRT deletion step is selectable it is possible to pool FIAU resistant clones and proceed immediately to the next step in parallel with clonal analysis. Alternatively it may be desirable to show by short range PCR that the human sequences are now adjacent to those of the mouse as shown (Hu-primer 1 and Mo-primer)

At this stage a 200 kb human locus will have been inserted.,

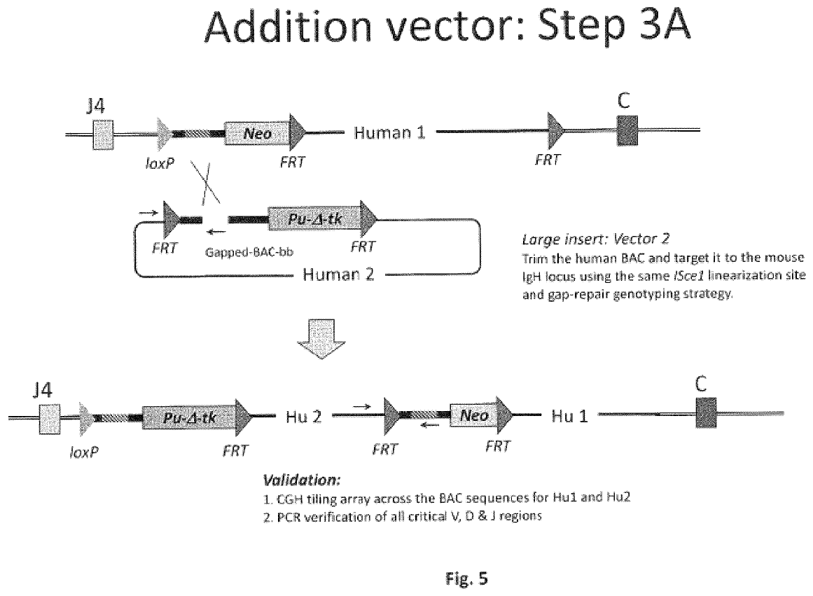

FIG. 5 illustrates a second large insert vector is targeted into the ES cell chromosome. The human BAC is targeted to the mouse IgH locus using the same initiation cassette insertion followed by ISce1 BAC linearization, BAC targeting to the initiation cassette and gap-repair genotyping strategy. Verification of the BAC insertion is carried out as before.

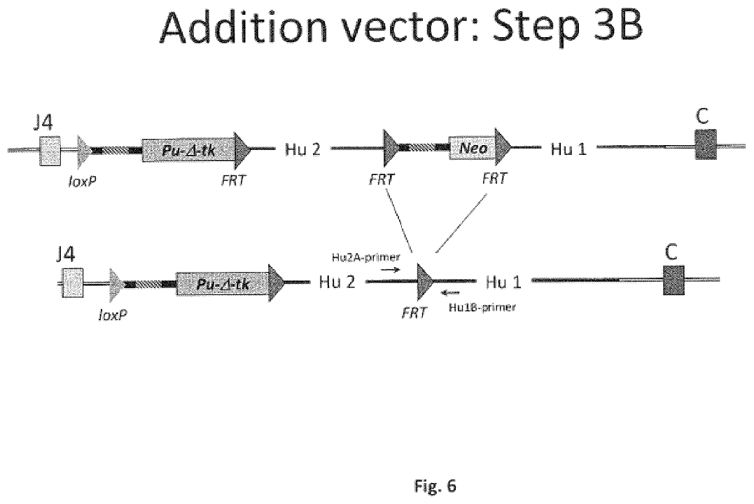

FIG. 6 illustrates the FRTY flanked BAC backbone of large insert vector 2 and the neo marker are deleted via Flpo. Note that this is not selectable, thus it will be necessary for clonal analysis at this point. This will enable confirmation of the juxtaposition of the human 2 insert with human 1 and other validation efforts.

At this stage a ˜200 kb human locus will have been inserted.

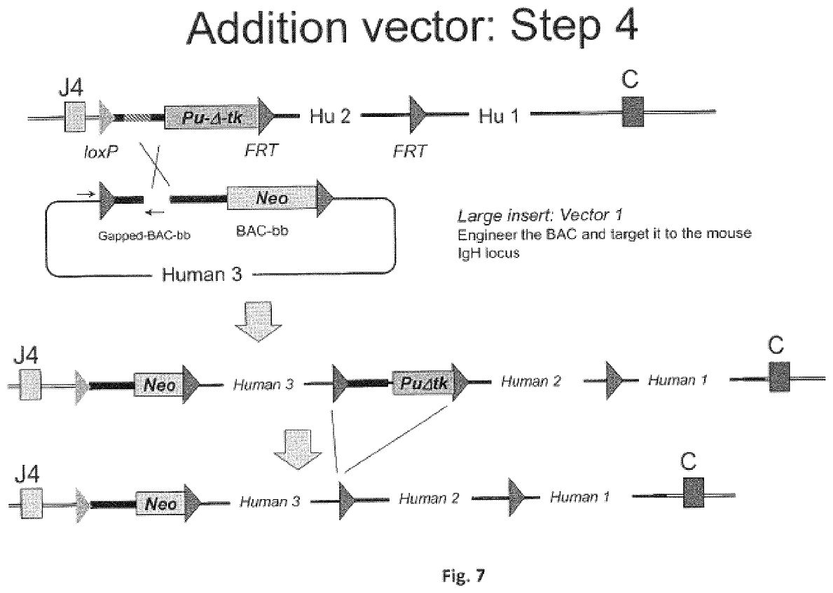

FIG. 7 illustrates the next large insert vector targeted to the mouse IgH locus. The pu-delta TK cassette is then removed, as for FIG. 4. The process can be repeated to incorporate other BACs.

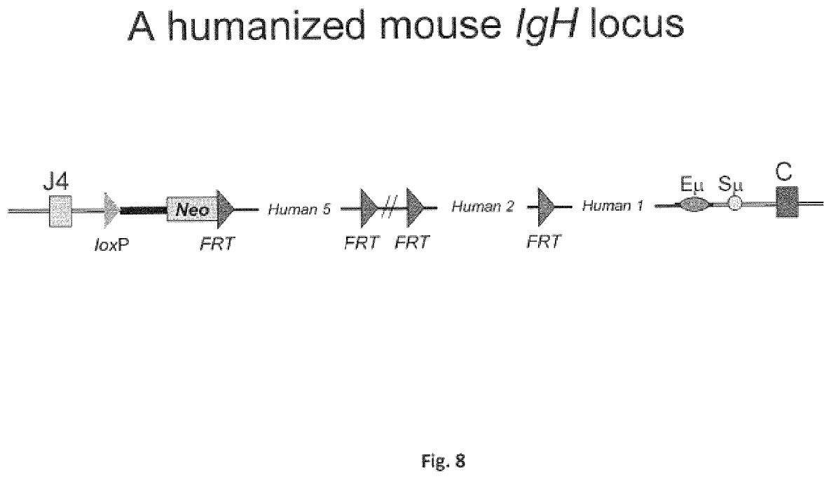

FIG. 8 illustrates the final predicted ES cell construct.

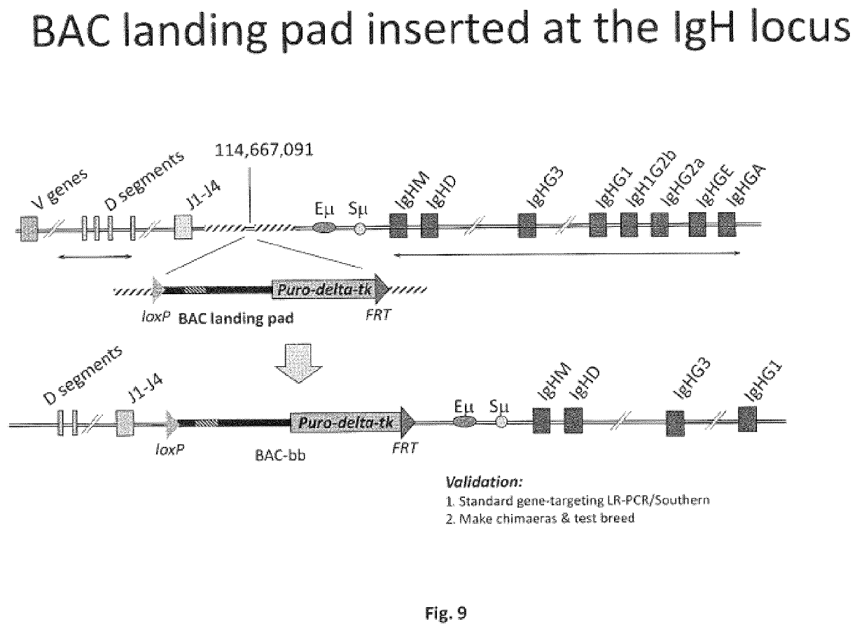

FIGS. 9-18 provide a further level of detail of this process.

### Example 2

Site-Specific Recombination

In a further method of the invention site specific recombination can also be employed. Site-specific recombination (SSR) has been widely used in the last 20-years for the integration of transgenes into defined chromosomal loci. SSR involves recombination between homologous DNA sequences.

The first generation of SSR-based chromosomal targeting involved recombination between (i) a single recombination target site (RT) such as IoxP or FRT in a transfected plasmid with (ii) a chromosomal RT site provided by a previous integration. A major problem with this approach is that insertion events are rare since excision is always more efficient than insertion. A second generation of SSR called RMCE (recombinase-mediated cassette exchange) was introduced by Schlake and Bode in 1994 (Schlake, T.; J. Bode (1994). Use of mutated FLP-recognition-target-(FRT-)sites for the exchange of expression cassettes at defined chromosomal loci“. Biochemistry 33: 12746-12751). Their method is based on using two heterospecific and incompatible RTs in the transfected plasmid which can recombine with compatible RT sites on the chromosome resulting in the swap of one piece of DNA for another—or a cassette exchange. This approach has been successfully exploited in a variety of efficient chromosomal targeting, including integration of BAC inserts of greater than 50 kb (Wallace, H. A. C. et al. (2007). “Manipulating the mouse genome to engineering precise functional syntenic replacements with human sequence”. Cell 128: 197-209; Prosser, H. M. et al. (2008). “Mosaic complementation demonstrates a regulatory role for myosin Vlla in actin dynamics of Stereocilia”. Mol. Cell. Biol. 28: 1702-12).

The largest insert size of a BAC is about 300-kb and therefore this places an upper limit on cassette size for RMCE.

In the present invention a new SSR-based technique called sequential RMCE (SRMCE) was used, which allows continuous insertion of BAC inserts into the same locus.

The method comprises the steps of
1 insertion of DNA forming an initiation cassette (also called a landing pad herein) into the genome of a cell;
2 insertion of a first DNA fragment into the insertion site, the first DNA fragment comprising a first portion of a human DNA and a first vector portion containing a first selectable marker or generating a selectable marker upon insertion;
3 removal of part of the vector DNA;
4 insertion of a second DNA fragment into the vector portion of the first DNA fragment, the second DNA fragment containing a second portion of human DNA and a second vector portion, the second vector portion containing a second selectable marker, or generating a second selectable marker upon insertion;
5 removal of any vector DNA to allow the first and second human DNA fragments to form a contiguous sequence; and
6 iteration of the steps of insertion of a part of the human V(D)J DNA and vector DNA removal, as necessary, to produce a cell with all or part of the human VDJ or VJ region sufficient to be capable of generating a chimaeric antibody in conjunction with a host constant region, wherein the insertion of at least one DNA fragment uses site specific recombination.

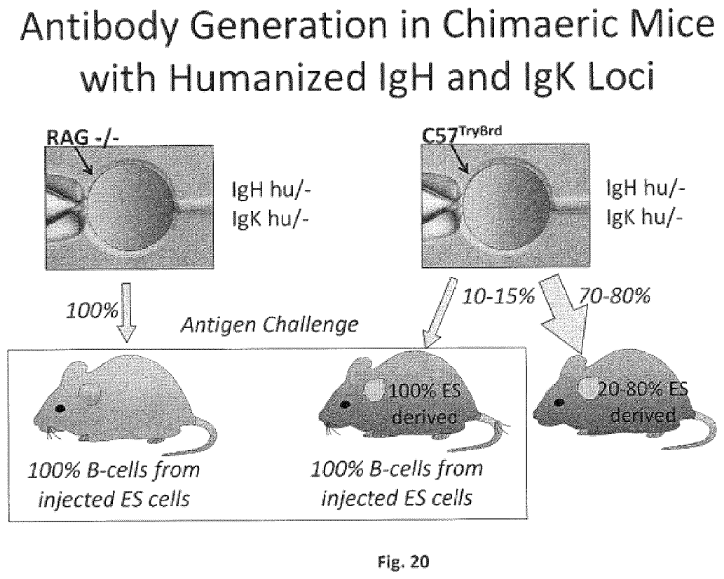

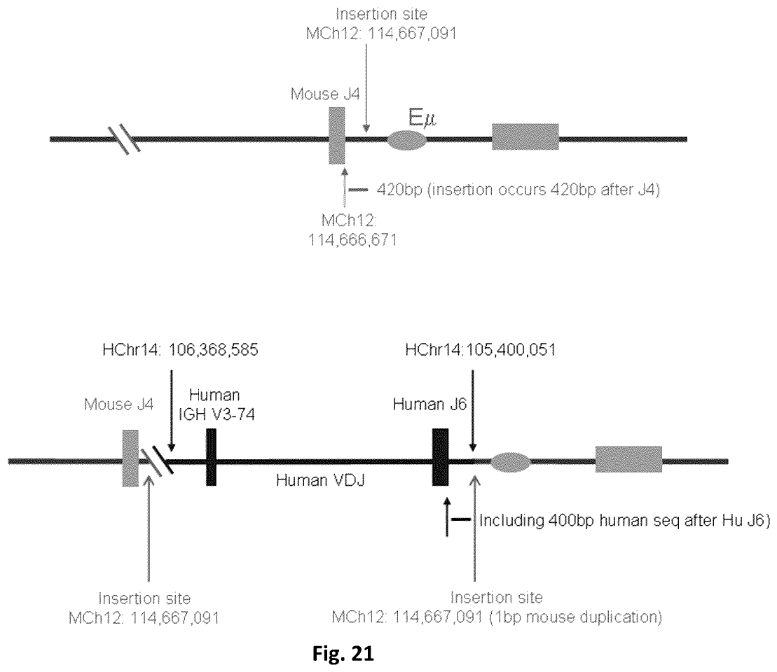

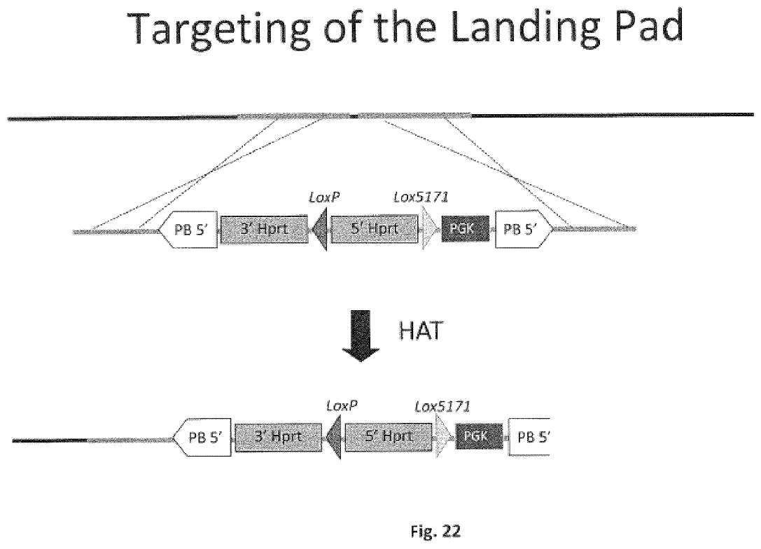

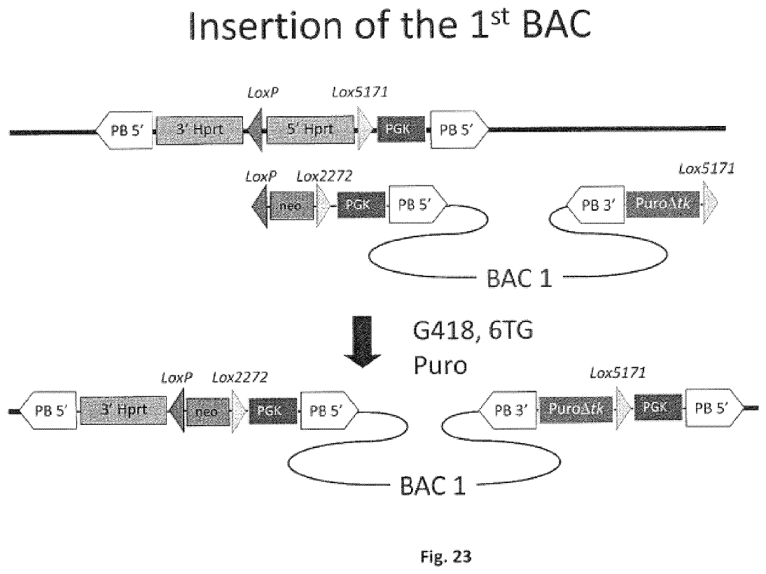

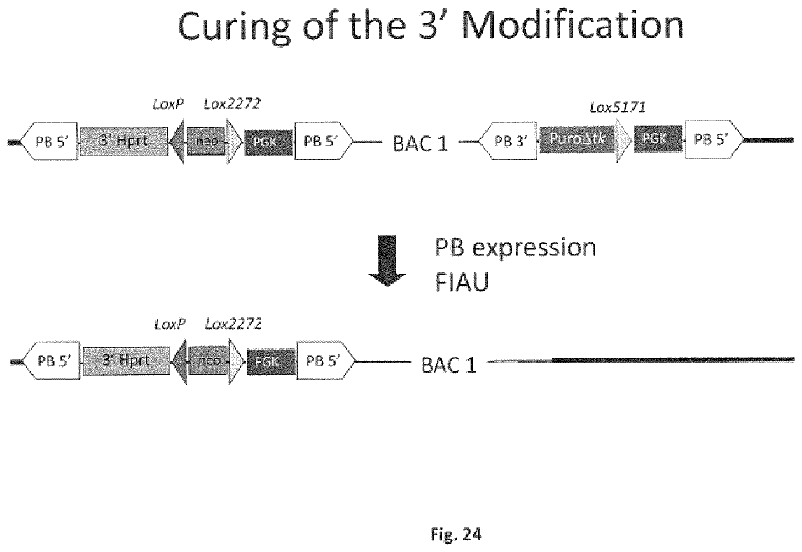

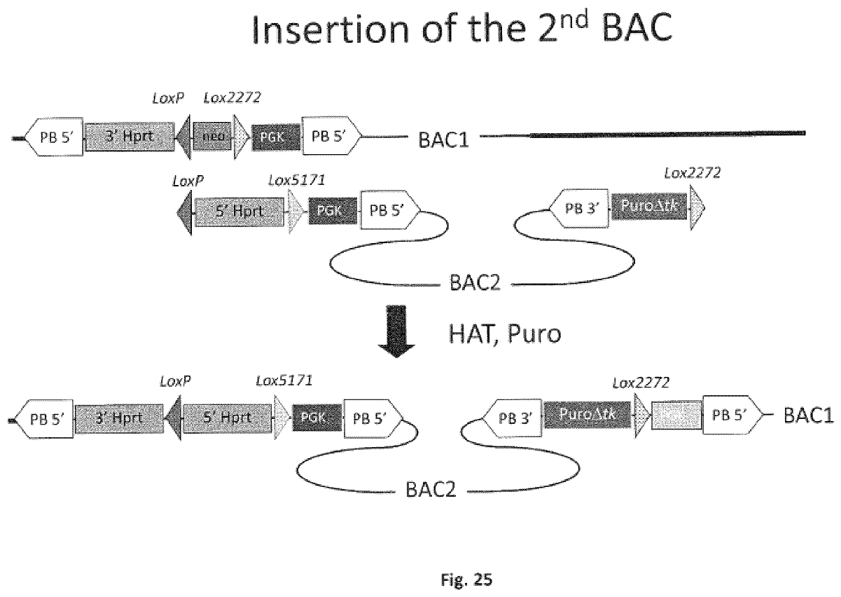

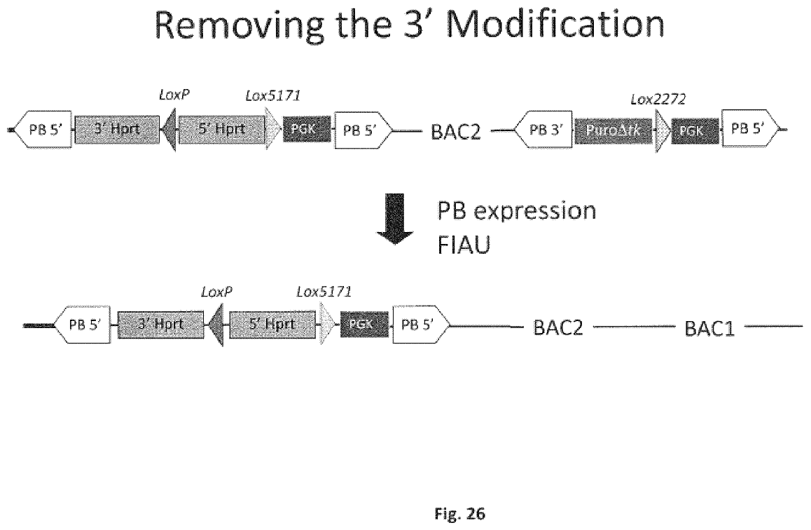

In one specific aspect the approach utilizes three heterospecific and incompatible loxP sites. The method is comprised of the steps as follows, and illustrated in FIGS. 22-26:
1. Targeting a landing pad into the defined locus. An entry vector containing an HPRT mini-gene flanked by inverted piggyBac (PB) ITRs is targeted into defined region (for example: a region between IGHJ and Eμ or IGKJ and Eκ or IGLC1 and EA3-1) to serve as a landing pad for BAC targeting. The HPRT mini-gene is comprised of two synthetic exons and associated intron. The 5′ HPRT exon is flanked by two heterospecific and incompatible loxP sites (one wild-type and the other a mutated site, lox5171) in inverted orientation to each other (FIG. 22). These two loxP sites provide recombination sites for the BAC insertion through RMCE.
2. Insertion of the 1st modified BAC into the targeted landing pad. The 1st BAC has a length of DNA to be inserted into the genome flanked by engineered modifications. The 5′ modification (loxP-neo gene-10)(2272-PGK promoter— PB 5′LTR) and 3′ modification (PB3′LTR-puroΔTK gene—lox5171) is depicted in FIG. 23 along with the relative orientations of the lox sites and PB LTRs. With transient CRE expression from a co-electroporated vector, the DNA sequence would be inserted into the defined locus through RMCE. The cells in which a correct insertion has occurred can be selected as follows: (i) Puromycin-resistance (the puroΔTK gene has acquired a promoter— “PGK”— from the landing pad), (ii) 6TG-resistance (the HPRT mini-gene has been disrupted), and (iii) G418-resistance (selects for any insertion via the 5′ region PGK-neo arrangement). Any combination of these selection regimes can be used. G418- and 6TG-resistance select for correct events on the 5′ end while puro-resistance selects for correct events on the 3′ end.
3. Curing (removing) the 3′ modification of the 1^{st} insertion. A properly inserted 1st BAC results the 3′ end having a puroΔTK gene flanked by inverted PB LTRs (FIG. 24)— essentially a proper transposon structure. This transposon can then be removed by the transient expression of the piggyBac transposase (from an electroporated vector). Cells with the correct excision event can be selected by FIAU resistance—ie, no thymidine kinase activity from the puroΔTK gene. This completely removes the 3′ modification leaving no trace nucleotides.
4. Insertion of a 2 nd modified BAC into the 5′ end of 1^{st} insertion. The 2^{nd} BAC has a length of DNA to be inserted into the genome (usually intended to be contiguous with the DNA inserted with the 1′t BAC) flanked by engineered modifications. The 5′ modification (loxP— HPRT mini gene 5′ portion −10)(5171— PGK promoter— PB5′LTR) and 3′ modification (PB3′LTR— puroΔTK −10x2272) is depicted in FIG. 25 along with the relative orientations of the lox sites and PB LTRs. With transient CRE expression from a co-electroporated vector, the DNA sequence would be inserted into the defined locus through RMCE. The cells in which a correct insertion has occurred can be selected as follows: (i) HAT-resistance (the HPRT mini-gene is reconstituted by a correct insertion event, ie: the 5′ and 3′ exon structures are brought together), and (ii) puromycin-resistance (puroΔTK gene has acquired a promoter— “PGK”— from the landing pad).
5. Curing (removing) the 3′ modification of the 2 ^{nd} insertion. A properly inserted 2^{nd} BAC results the 3′ end having a puroΔTK gene flanked by inverted PB LTRs (FIG. 26)— essentially a proper transposon structure, exactly analogous to the consequence of a successful 1st BAC insertion. And therefore this transposon can likewise be removed by the transient expression of the piggyBac transposase (from an electroporated vector). Cells with the correct excision event can be selected by FIAU resistance—ie, no thymidine kinase activity from the puroΔTK gene. This completely removes the 3′ modification leaving no trace nucleotides.
6. After curing of the 3′ modification of the 2 ^{nd} BAC insertion, the landing pad becomes identical to the original. This entire process, steps 2 through 5, can be repeated multiple times to build up a large insertion into the genome. When complete, there are no residual nucleotides remaining other than the desired insertion.

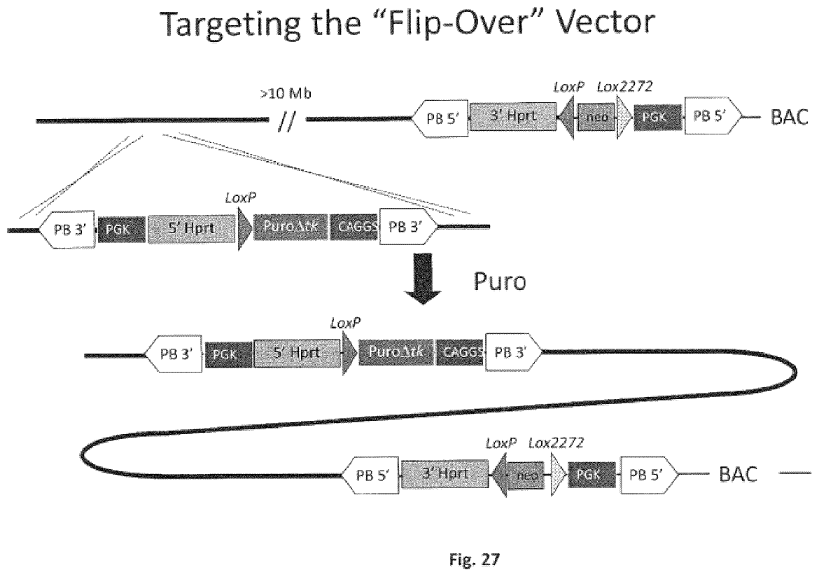

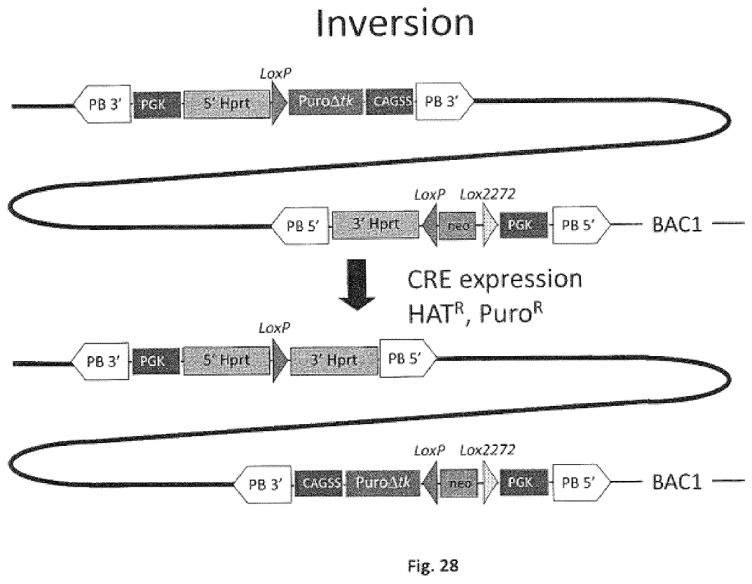

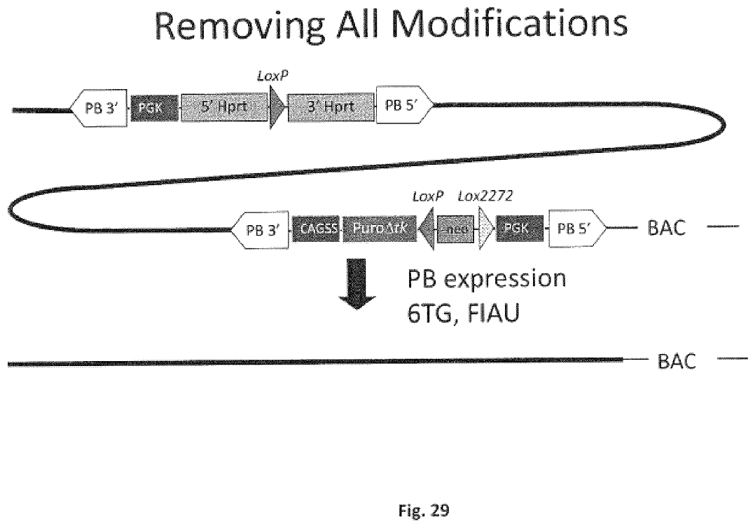

With the insertion of an odd number of BACs into the Ig loci, the endogenous VDJ or VJ sequences can be inactivated through an inversion via chromosomal engineering as follows (see FIGS. 27-29):
1. Targeting a “flip-over” cassette into a 5′ region 10 to 40 megabases away from the endogenous VDJ or VJ. The flip-over vector (PB3′LTR— PGK promoter— HPRT mini gene 5′ portion—loxP puroΔTK CAGGS promoter— PB3′LTR) is depicted in FIG. 27 along with the relative orientations of the lox sites and PB LTRs.
2. Transient CRE expression will result in recombination between the loxP site in the “flip-over” cassette and the loxP site in the 5′ modification. This 5′ modification is as described in Steps 2 and 3 above—essentially the modification resulting from insertion of an odd number of BACs, after the 3′ modification has been cured. The loxP sites are inverted relative to one another and therefore the described recombination event results in an inversion as depicted in FIG. 28. Cells with the correct inversion will be HAT-resistance since the HPRT mini-gene is reconstituted by a correct inversion.
3. A correct inversion also leaves two transposon structures flanking the “flip-over” cassette and the 5′ modification. Both can be excised with transient piggyBAC transposase expression, leaving no remnant of either modification (FIG. 29). Cells with the correct excisions can be selected as follows: (i) 6TG-resistance (the HPRT mini-gene is deleted) and (ii) FIAU-resistance (the puroΔTK gene is deleted). An inversion as described in the Ig loci would move the endogenous IGH-VDJ or IGK-VJ region away from the Eμ or Eκ enhancer region, respectively, and lead to inactivation of the endogenous IGH-VDJ or IGK-VJ regions.

The methods of insertion of the invention suitably provide one or more of:
Selection at both 5′ and 3′ ends of the inserted DNA fragment;
Efficient curing of the 3′ modification, preferably by transposase mediated DNA excision;
Inactivation of endogenous IGH or IGK activity through an inversion; and
Excision of modifications, leaving no nucleotide traces remaining in the chromosome.

### Example 3

Insertion of a Test Vector Into the Genome at a Defined Location

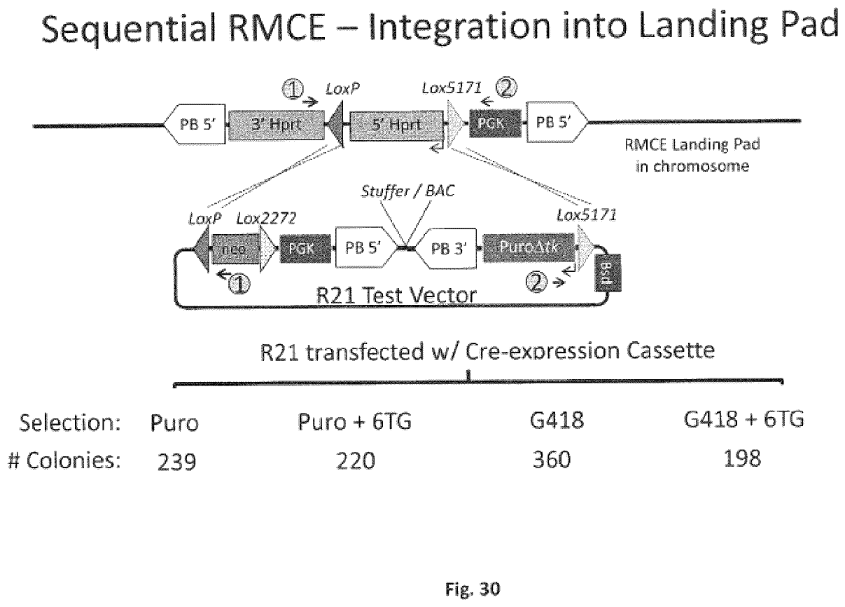

Proof of concept of the approach is disclosed in FIG. 30. In FIG. 30 a landing pad as shown in FIG. 22 was inserted into the genome of a mouse by homologous recombination, followed by insertion of the R21 plasmid into that landing pad via cre-mediated site specific recombination. The insertion event generated a number of general insertion events, 360 G418 resistant colonies, of which ˜220 were inserted into the desired locus, as demonstrated by disruption of the HRPT minilocus.

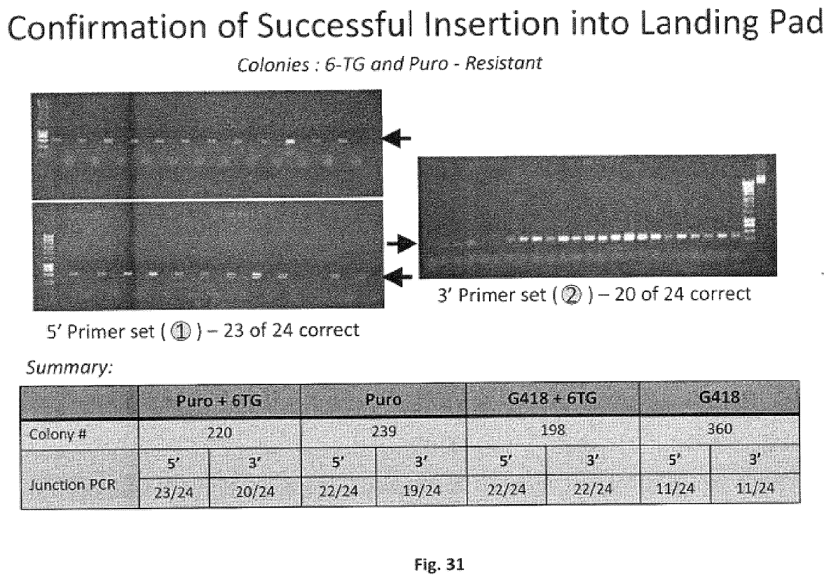

The R21 vector mimicks the 1^{st} BAC insertion vector at the 5′ and 3′ ends, including all selection elements and recombinase target sites. In place of BAC sequences, there is a small ‘stuffer’ sequence. This vector will both test all the principals designed in the invention and allow easy testing of the results in that PCR across the stuffer is feasible and therefore allows both ends of the insertion to be easily tested. R21 was co-electroporated with a cre-expressing vector into the ES cells harbouring the landing pad in the IGH locus. Four sets of transformed cells were transfected in parallel and then placed under different selection regimes as indicated in FIG. 30. G418 selection (neo gene expression) resulted in the largest number of colonies due to there being no requirement for specific landing-pad integration. Any integration of R21 into the genome will provide neo expression leading to G418-resistance. Pura selection resulted in a similar colony number to Pura+6TG or G418+6TG, suggesting that the stringency of Puro selection is due to the PuroΔTK lacking a promoter in the vector. Pura expression is only acquired when an integration occurs near a promoter element—in this design most likely specifically in the landing pad. These conclusions are supported by the results from junction PCR which is shown in FIG. 31.

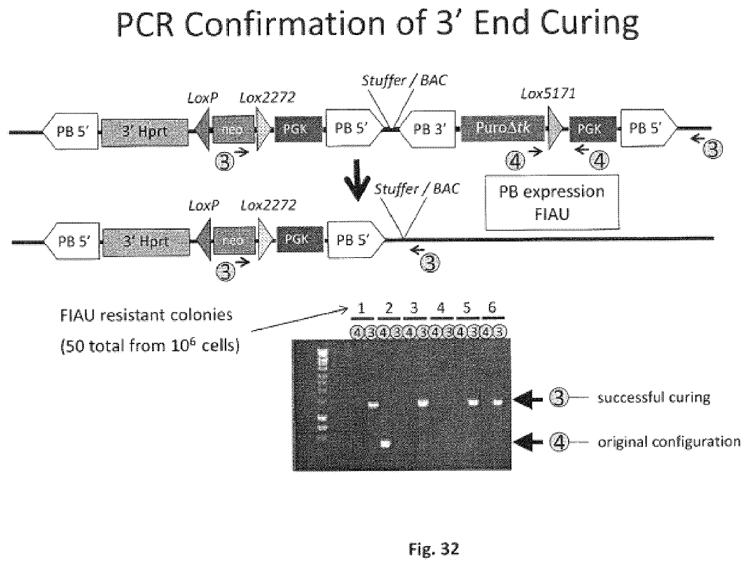

The next step in the invention is to ‘cure’ the 3′ end of the integrated BAC vector, leaving a seamless transition between the insertion and the flanking genome. This curing was demonstrated by expanding an individual clone from above (R21 inserted into the landing pad) and expressing piggyBac recombinase in this clone via transfection of an expressing plasmid. FIAU was used to select colonies in which the 3′ modification was excised—ie, through loss of the PGK-puroΔTK′ element between the piggyBac terminal repeats. Fifty such clones resulted from a transfection of 10⁶ cells; of these six were tested for the expected genomic structure. Successful curing resulted in positive FOR between the primer set labelled “3” in FIG. 32. Of the 6 clones, 4 had correct excisions, 1 clone remained in the original configuration and 1 other had a deletion.

These data demonstrate iterative insertion of DNA into a landing pad at a defined genomic locus using the approaches outlined above.

### Example 4

Insertion of Large Parts of the Human IG Loci Into Defined Positions in the Mouse Genome

Example 3 demonstrated that the design of the claimed invention was capable of providing for the insertion of a test vector into the genome at a defined location, in this case the R21 vector into the mouse IGH locus. The use of the appropriate selection media and the expression of cre-recombinase resulted in a genomic alteration with the predicted structure.

The same design elements described in this invention were built into the 5′ and 3′ ends of a BAC insert. Said insert comprised human sequences from the IGH locus and was approximately 166-kb. This engineered BAC was electroporated along with a cre-expressing plasmid DNA into mouse ES cells harbouring the landing pad at the mouse IGH locus. The transfected cell population was grown in puro-containing media to select for appropriate insertion events.

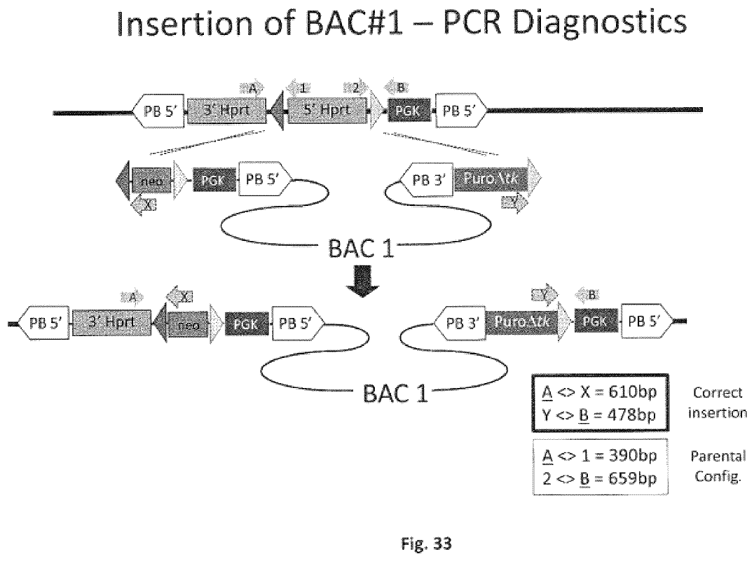

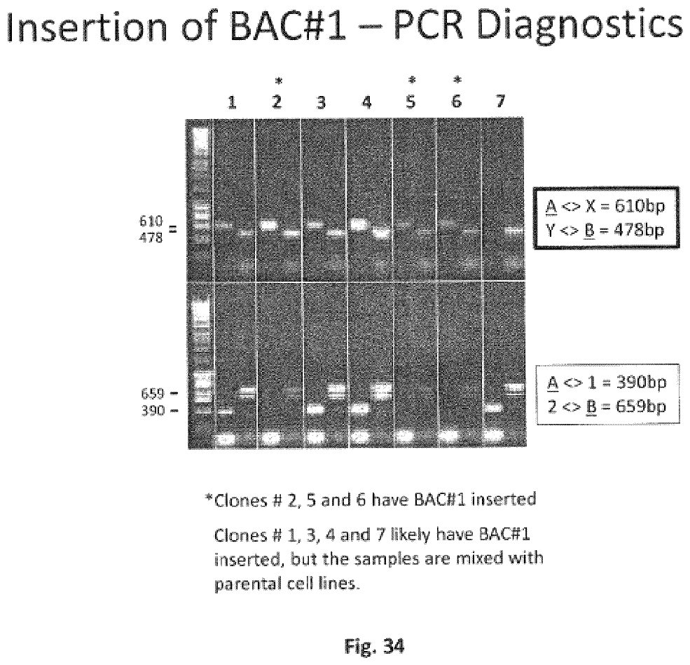

Seven resulting clones were isolated and further analysed. The expected recombination event and resulting structure are depicted in FIG. 33. Based upon data from the R21 experiment outlined in Example 3, a stringent selection for correct clones was expected when the transfected population was selected in puro-containing media. This is because the puro-coding region requires a promoter element and this is preferentially supplied by the landing pad after recombination. Accordingly, the majority of the 7 isolated clones had inserted correctly into the genome at the landing pad as determined by the diagnostic PCR. The primers for diagnosing a correct insertion are depicted in FIG. 33. Correct junctions are present in the genome if a 610-bp fragment is amplified between primers ‘A’ and ‘X’ and a 478-bp fragment is amplified between primers ‘Y’ and ‘B’ (FIGS. 33 and 34). Note that there are amplified fragments between ‘A’ and ‘1’ primers and ‘2’ and ‘B’ primers indicating the presence of parental genome (that is, the landing pad alone). These result from parental cells present internally in the cell colonies under puro-selection that escape the selection due to the geometry of a colony. After passaging the colony through puro-containing media, these parental junction fragments disappear indicating that the parental cells are removed from the population. In addition, all the clones were shown to be resistant to 6-TG as expected if the HPRT gene is inactivated by the correct insertion event.

These data indicate that the disclosed strategy for inserting large parts of the human IG loci into defined positions in the mouse genome will enable the construction of a mouse with a plurality of the variable regions of human IG regions upstream of the mouse constant regions as described.

### Example 5

Inserted Loci Are Functional in Terms of Gene Rearrangement, Junctional Diversity as Well as Expression

Bacterial artificial chromosomes (BACs) were created, wherein the BACs had inserts of human Ig gene segments (human V, D and/or J gene segments). Using methods described herein, landing pads were used in a method to construct chimaeric Ig loci in mouse embryonic stem cells (ES cells). such that chimaeric IgH and IgK loci were provided in which human gene segments are functionally inserted upstream of endogenous constant regions. To test if the human IgH-VDJ or IgK-VJ gene segments in the chimaera mice derived from human BAC-inserted ES cell clones appropriately rearrange and express, RT-PCR was performed for the RNA samples of white blood cells from those mice with the primer pairs of human variable(V) region and mouse constant(C) region. The sequences of oligos are shown as follows (Table 1). Each V oligo is paired with C oligo (HV with Cμ; KV with C↓) for FOR reaction.

TABLE 1

| Oligo | Sequence |
|---|---|
| HV2-5 | AGATCACCTTGAAGGAGTCTGGTCC (SEQ ID NO 7) |
| HV4-4 | TGGTGAAGCCTTCGGAGACCCTGTC (SEQ ID NO 8) |
| HV1-3 | CACTAGCTATGCTATGCATTGGGTG (SEQ ID NO 9) |
| HV1-2 | ATGGATCAACCCTAACAGTGGTGGC (SEQ ID NO 10) |
| HV6-1 | GGAAGGACATACTACAGGTCCAAGT (SEQ ID NO 11) |
| Cμ | TAGGTACTTGCCCCCTGTCCTCAGT (SEQ ID NO 12) |
| KV1-9 | AGCCCAGTGTGTTCCGTACAGCCTG (SEQ ID NO 13) |
| KV1-8 | ATCCTCATTCTCTGCATCTACAGGA (SEQ ID NO 14) |
| KV1-6 | GGTAAGGATGGAGAACACTGGCAGT (SEQ ID NO 15) |
| KV1-5 | TTAGTAGCTGGTTGGCCTGGTATCA (SEQ ID NO 16) |
| Cκ | CTTTGCTGTCCTGATCAGTCCAACT (SEQ ID NO 17) |

Using the one-step formulation of SuperScript™ III One-Step RT-PCR System with Platinum@ Taq High Fidelity (Invitrogen™; World Wide Web (www) invitrogen.comisite/us/en/homal

References/protocols/nucleic-acid-amplification-and-expression-profiling/per-protocolisuperscript-3-one-step-rt-per-system-with-platinum-taq-high-fidelity.html #prot3), both cDNA synthesis and PCR amplification were achieved in a single tube using gene-specific primers and target RNAs.

The RT-PCR results showed most of the human IGH-VDJ or IGK-VJ gene segments appropriately rearrange and express in the chimaera mice. To investigate the details about the diversity generated from VDJNJ rearrangement, those specific RT-PCR fragments were cloned into a common vector for sequencing..

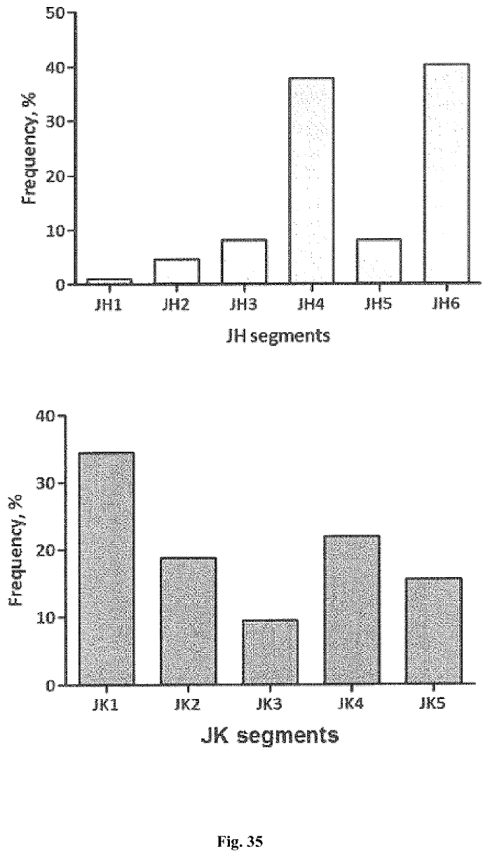

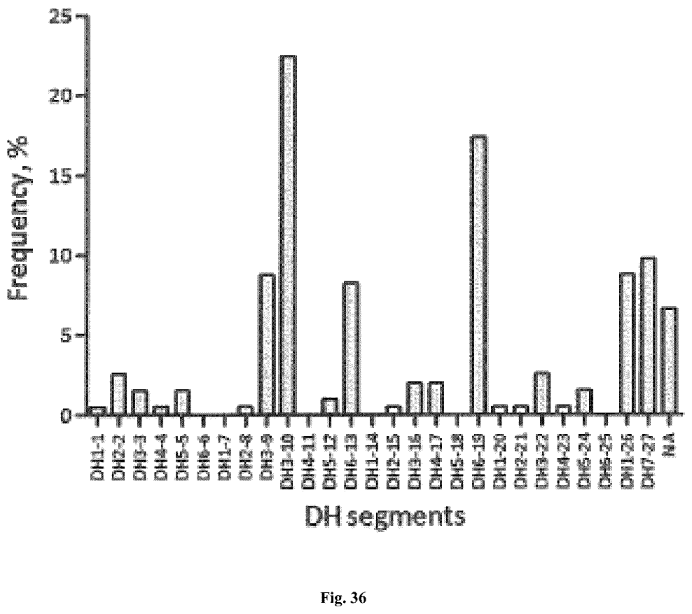

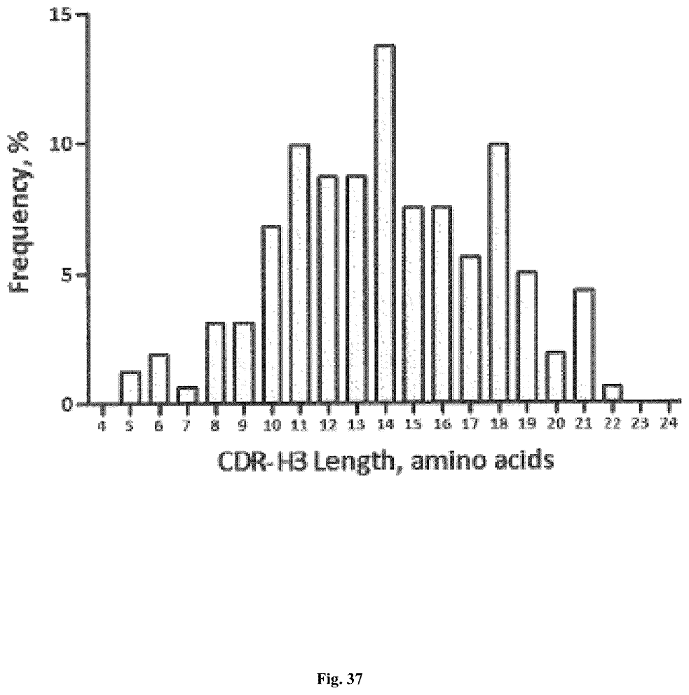

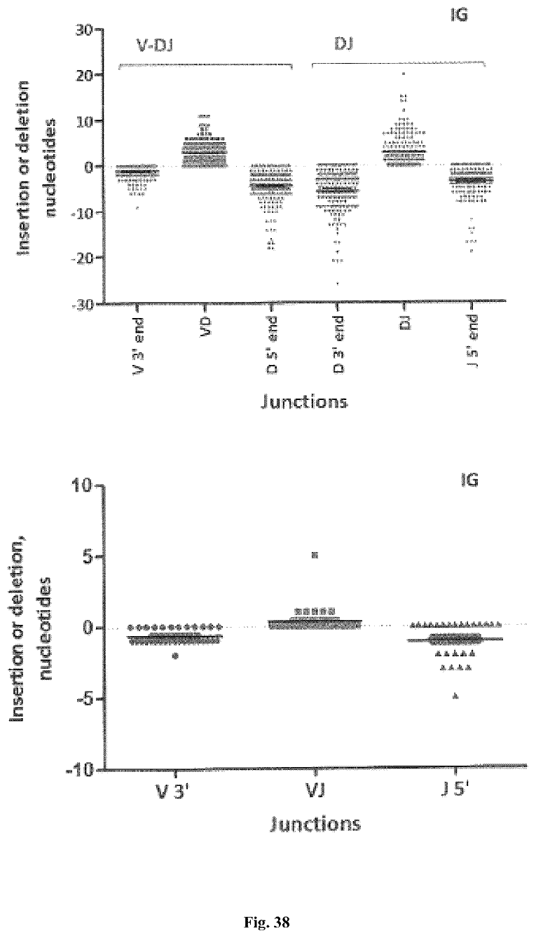

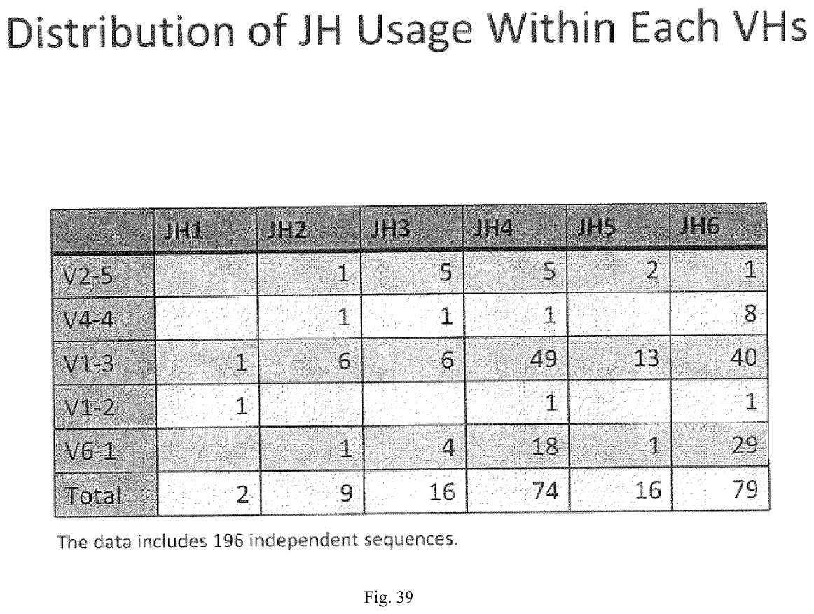

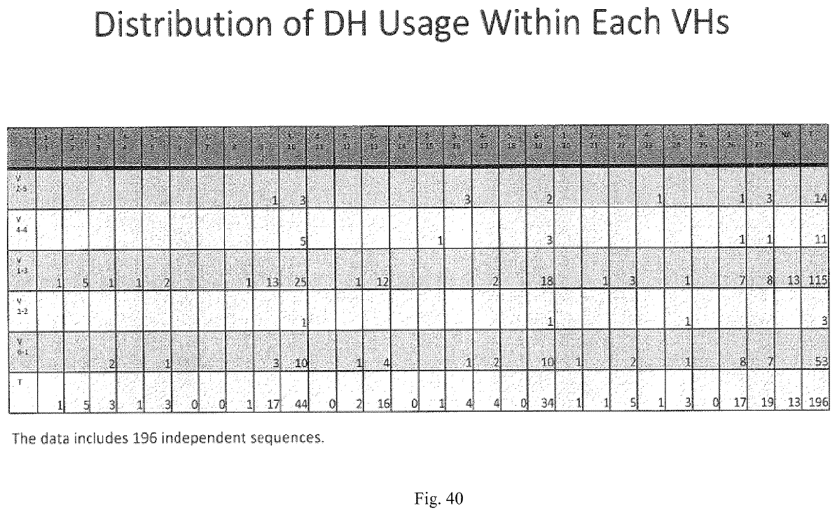

Sequencing results indicate that JH, DH, and JK usages (FIG. 35 and FIG. 36) are similar to human results. In addition, the results from the IGH-VDJCμ transcripts show that the range and mean of CDR-H3 length (FIG. 37) are similar to that observed in human. The junctional diversity generated from exonuclease and nucleotide addition activities (FIG. 38) was also observed. The IGH rearrangement possessed a higher frequency of these activities compared to the IGK one. These data suggest that the inserted loci are functional in terms of gene rearrangement, junctional diversity as well as expression.

### Example 6

Productive VJ Rearrangement and Somatic Hypermutation can be Obtained

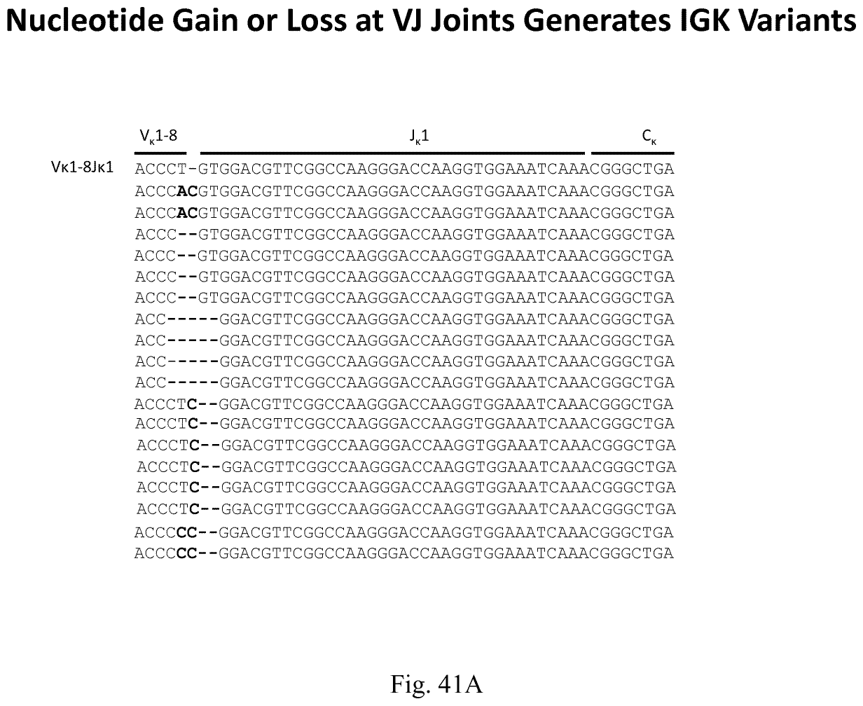

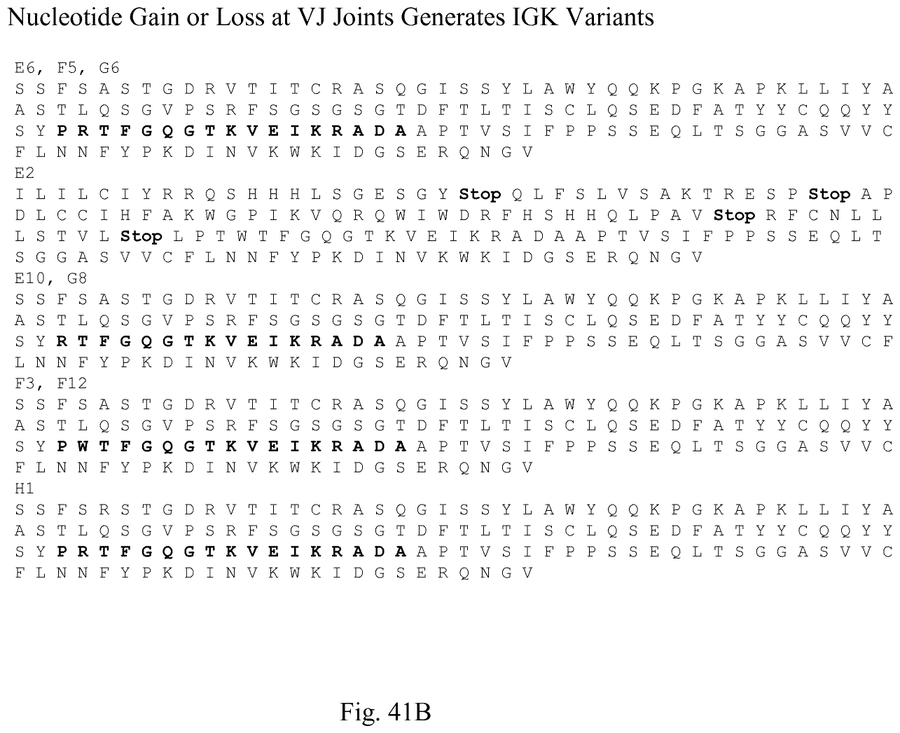

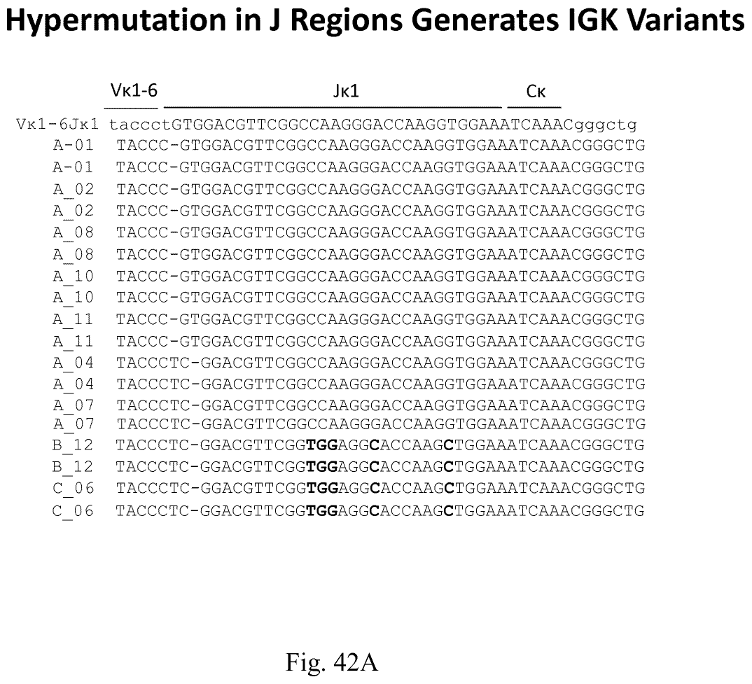

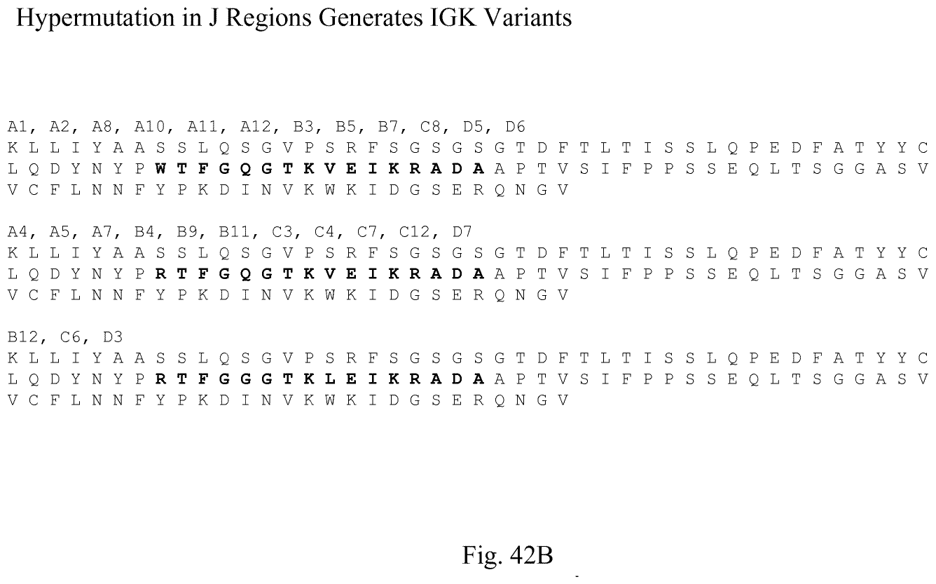

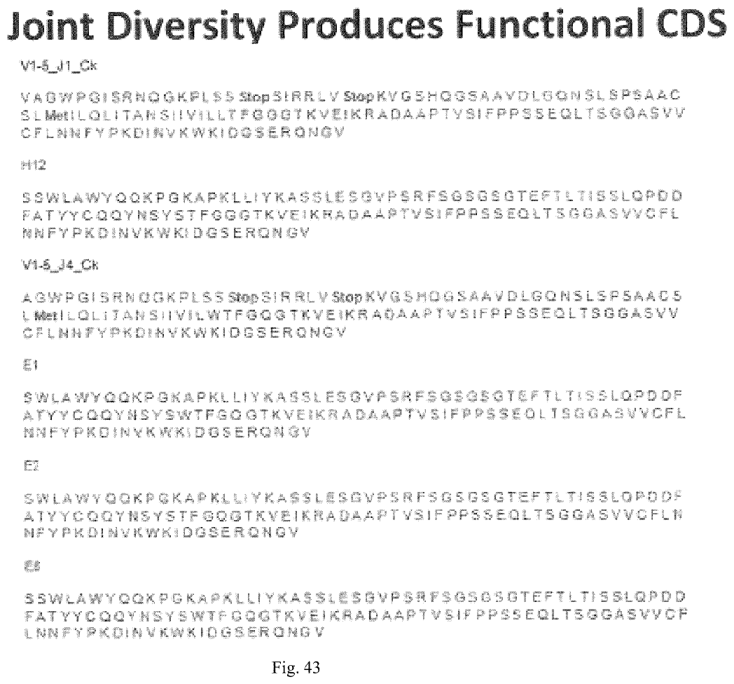

FIG. 41 shows an analysis of kappa mRNA from mice B-cells bearing rearranged VJ, the VJ having been rearranged from human germline kappa V1-8 and J1, and demonstrates that both that productive VJ rearrangement and somatic hypermutation can be obtained, the latter as seen from the changes in antibodies encoded by mRNA with respect to the germline sequences. The same is displayed for V1-6 and J1 in FIG. 42. Importantly, the recombination eliminates stop codons that are encoded by the combination of (unmutated) human germline gene segments, thereby allowing for antibody-encoding mRNA sequences. FIG. 43 demonstrates that inserted human kappa V1-5 J1 and V1-5 J4 can produce functional coding sequences in vivo and junctional diversity.

### Example 7

Inactivation of Use of Endogenous IGHV Gene Segments for Expressed Rearranged Heavy Chain by Inversion

### INTRODUCTION

A 5′-RACE Cμ-specific library was generated from the splenic B lymphocytes of transgenic mice, denoted S1 mice. These mice comprise transgenic heavy chain loci, each locus containing the six most 3′ functional human V_{H} gene segments (V_{H}2-5, 7-4-1, 4-4, 1-3, 1-2, 6-1), and all the human D and J_{H} gene segments inserted into the endogenous heavy chain locus between endogenous

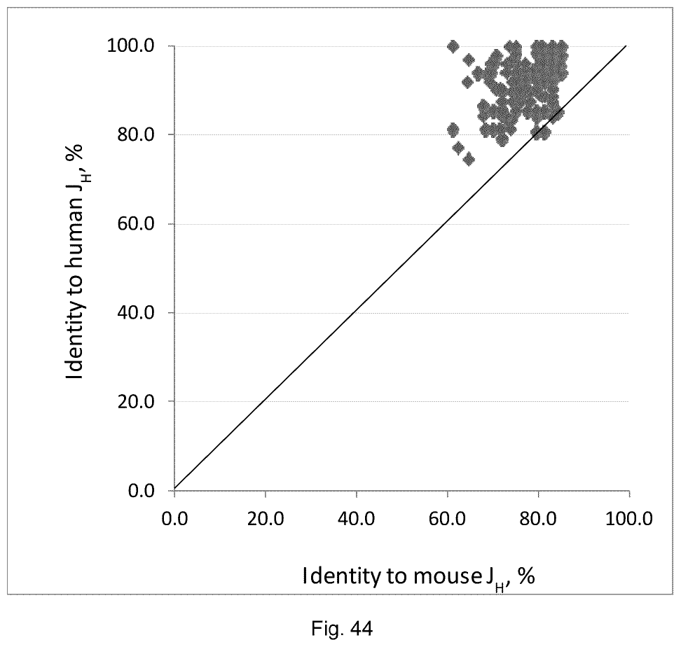

IGHJ4 and Eμ (mouse chromosome 12: between coordinates 114666435 and 114666436). The human DNA was obtained from a bacterial artificial chromosome (BAC) containing the sequence of human chromosome 14 from coordinate 106328951 to coordinate 106494908. Further details on the construction of transgenic antibody loci using sRMCE is given elsewhere herein and in 102011004192 (which is incorporated herein by reference). 4x96-well plates of clones were randomly picked for sequencing to determine the usage of the gene segments. All detected immunoglobulin heavy chains were rearranged from mouse V_{H} or human V_{H} with human D-J_{H}. No mouse D and J_{H} segments were detected in rearranged products (FIG. 44).

This result indicates that insertion of human V_{H}-D-J_{H} gene segments into an endogenous locus between the last endogenous J region (in this case, J_{H}4) and the Eμ enhancer effectively inactivates the use of endogenous D and J_{H} gene segments for expressed rearranged immunoglobulin heavy chains.

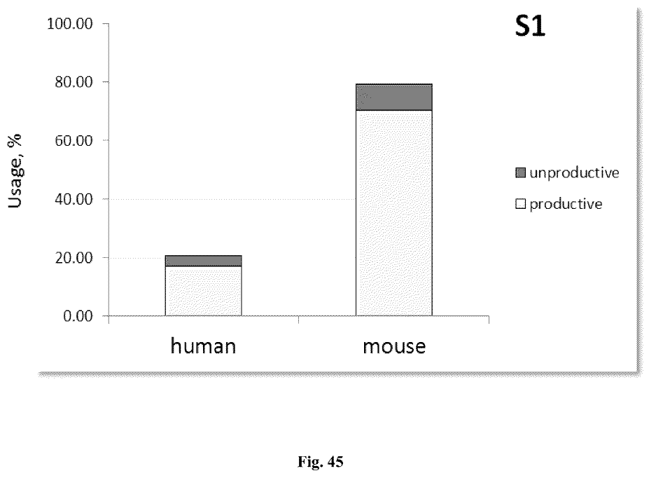

The ratio of mouse V_{H} to human V_{H} usage was around 3 to 1 (FIG. 45). To completely eliminate mouse V_{H} use for antibody generation, the endogenous mouse V_{H}-D-J_{H} was inverted and moved to a distant region of the same chromosome. The rearrangement of mouse VHs to human D-J_{H} segments was totally blocked by effects of inversion and distance from the heavy chain locus.

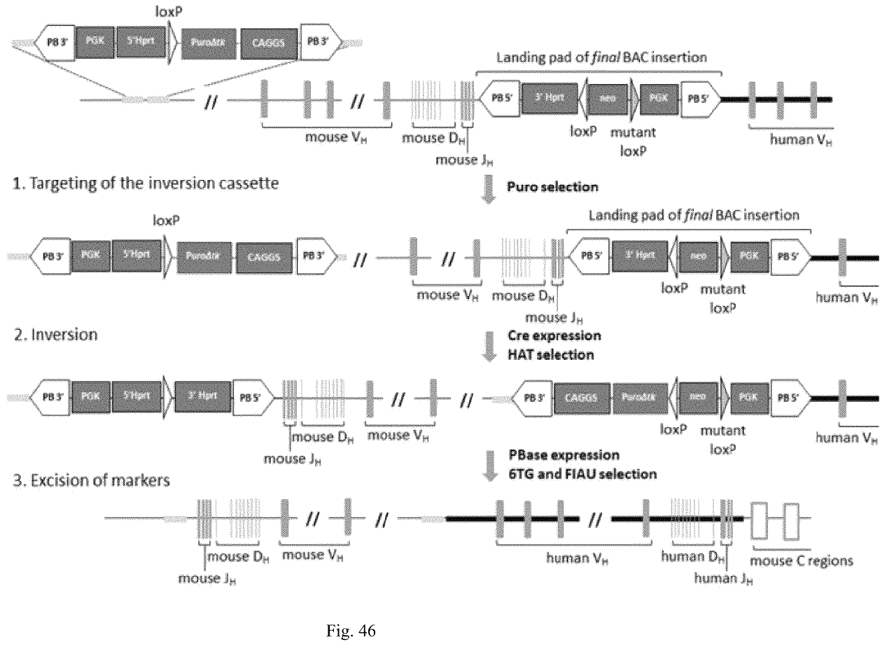

The inversion strategy included three steps: (a) targeting of an inversion cassette, (b) inversion of endogenous VDJ and (c) excision of markers (FIG. 46).

(a) Targeting of the Inversion Cassette:

The inversion cassette consists of four components: a CAGGS promoter-driven puromycin-resistant-delta-thymidine kinase (puroΔtk) gene, a 5′ HPRT gene segment under the PGK promoter control, a loxP site between them and inversely oriented to another loxP site already in the heavy chain locus, and two flanking piggyback LTRs (PB3′LTRs). The inversion targeting cassette was inserted to a region that is 5′ and distant to the endogenous IGH locus at chromosome 12 as shown in FIG. 46. The targeted ES clones were identified and confirmed by PCR.

(b) Inversion:

Following the insertion, transient expression of cre from a transfected plasmid resulted in inversion of a section of chromosome 12 fragment including the endogenous V_{H}-D-J_{H} locus and intervening sequences through recombination of two inverted loxP sites, ie, those in the inversion cassette and the landing pad for the BAC insertion respectively. The invertants were selected by HAT and confirmed by junction PCRs cross the two recombined loxP sites.

(c) Excision of Markers:

The inversion rearranged the relative orientation of the PB3′LTRs from the inversion cassette and PB51TR from the landing pad to generate two piggyBac transposon structures flanking the inverted region. With transient expression of piggyBac transposase (PBase), these two transposons were excised from the chromosome (and thus the mouse cell genome). The cured ES clones were selected by 1-(-2-deoxy-2-fluoro-1-b-D-arabinofuranosyl)-5-iodouracil (FIAU) and 6TG, and confirmed by junction PCRs cross the excised regions.

Methods

Tissue culture: The procedures for ES cell culture, electroporation and drug selection have been described previously (Ramirez-Solis, R., A. C. Davis, and A. Bradley. 1993. Gene targeting in mouse embryonic stem cells. Methods Enzymol. 225:855-878).

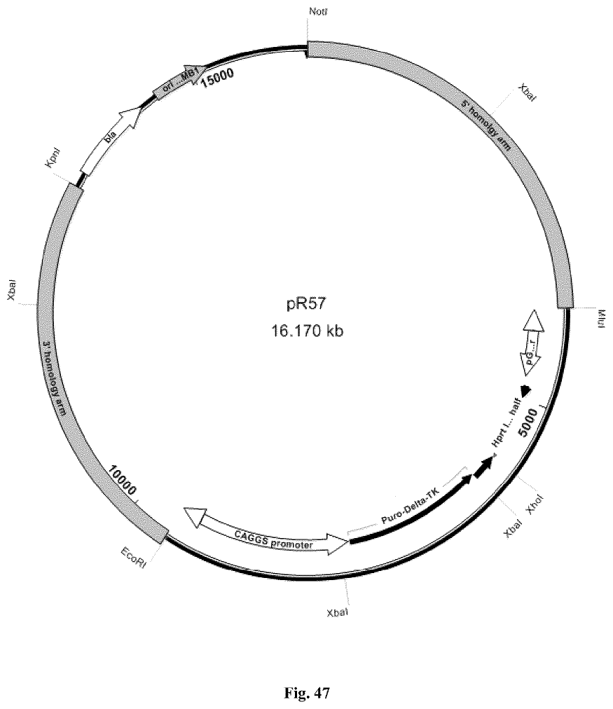

Targeting of the locus for inversion: Briefly, S1 cell line (S1.11,1) was cultured in M15 medium (Knockout™ DMEM supplemented with 15% fetal bovine serum, 2 mM glutamine, antibiotics, and 0.1 mM 2-mercaptoethonal). Targeting construct R57 (FIG. 47) was linearized outside the region of homology by Notl. A total of 20 μg of the linearized construct was electroporated into S1 cell lines (AB2.1-derived) with a Bio-Rad® Gene Pulser™, and 107 cells were plated onto three 90-mm-diameter SNL76/7 feeder plates containing M15 medium. At 24 h after electroporation, M15 containing puromycin (3 μg of the active ingredient per ml) was added to each 90-mm-diameter plate, and the cells were maintained under selection for 9 days. 96 puromycin-resistant clones were then picked and expanded in 96-well plates. The targeting events were identified by long-range PCR.

Cre-IoxP mediated inversion: 12 positive clones were pooled together and cultured in a 6-well tissue culture plate with M15 medium. The cells were transfected with 10 μg of pCAGGS-Cre plasmid for the inversion of mouse endogenous locus and then plated onto three 90-mm-diameter SNL76/7 feeder plates containing M15 medium. At 24 h after electroporation, M15 containing 1XHAT (hypoxanthine-aminopterin-thymidine) was added to each 90-mm-diameter plate, and the cells were maintained under selection for 7 days and then treated with 1XHT (hypoxanthine-thymidine) for 2 days. 48 HAT resistant colonies were picked and genotyped by PCR amplification of the junctions after Cre-loxP mediated inversion.

HyPBase-mediated marker excision: 12 positive clones were pooled together and cultured in 6-well tissue culture plate using M15 medium. The cells were transfected with 5 μg of HyPBase plasmid to activate the PB transposon LTRs flanking two selection markers (Hprt-mini gene and PGK-puroΔtk gene) and plated onto one 90-mm-diameter SNL76/7 feeder plates containing M15 medium. At 72 h after electroporation, a serial dilution of the cells was then plated onto three 90-mm-diameter SNL76/7 feeder plates containing M15 supplemented with 1+2-deoxy-2-fluoro-1-b-D-arabinofuranosyl)-5-iodouracil (FIAU). Cells were maintained under selection for 10 days, and FIAU-resistant colonies were counted, picked, and expanded in 96-well plates. Positive clones were identified by PCR amplification of the junctions after excision of the selection markers. Positive clones were then expanded for blastocyst microinjection.

Generation of chimera and breeding: Mouse chimaeras were generated by microinjection of ES cells into C57iBL6 blastocysts and transferred into pseudopregnant recipients. Male chimaeras were test-crossed with C57iBL6 mice. Agouti Fi offspring were genotyped by S1 3′ junction PCR. Test-cross positive heterozygotes were further intercrossed to generate homozygotes.

Determination of VH-D-JH usage by rapid amplification of 5′-cDNA ends (5′ RACE) PCR: Total RNA was extracted from the spleen of S1 inv1 mouse (KMSF30.1d) with TRIzol® Reagent (Invitrogen™, Life Technologies Ltd™) and treated with DNase I. Rapid amplification of 5′-cDNA ends (5′ RACE) FOR was performed using 5′/3′ RACE kit (2^{nd} Generation, Roche) following the protocol supplied by the manufacturer. The first-strand cDNA was synthesised using primer E1554 (5′-ATGACTTCAGTGTTGTTCTGGTAG-3′; SEQ ID No 25) which is located at the mouse endogenous Cμ region. The synthesised first cDNA strand was purified using High Pure PCR Product Purification Kit (Roche). Poly(A) tail was added following the protocol supplied with the 573′ RACE kit (2^{nd} Generation, Roche). The 5′ end of the V_{H}-D-J_{H} rearranged transcript was amplified by nested FOR with forward primers Oligo dT, which is included in the kit, and nested Cμ-specific reverse primers E1555 (5′-CACCAGATTCTTATCAGAC-3′; SEQ ID No 26). Following reaction, the 5′ RACE FOR product was checked on a 1% agarose gel and purified using QIAquicke Gel Extraction Kit (QIAGEN) as the protocol supplied with the kit, then cloned into pDrive vector using QIAGEN PCR Cloning Kit (QIAGEN) for sequencing analysis.

Results

The sequence analysis from a Cμ-specific 5′-RACE library of splenic B lymphocytes of S1^{inv1} (one human IGH BAC (ie, multiple human V_{H}, all functional human D and JH) with an inverted endogenous IGH locus version 1) mouse shows that practically all the transcripts came from rearranged human V_{H}-D-J_{H} gene segments (FIG. 48). Mouse V_{H} usage was rarely detected (0.4%), and no mouse D and J_{H} usage was detected. Human V_{H}. usage was 99.6% and only human D and J_{H} were used; it was hypothesized that the rare mouse V_{H} usage was due to trans-switching with another chromosome and not due to use of moue V_{H} from the inverted sequences. The inversion resulted in complete inactivation of the endogenous V_{H} use.

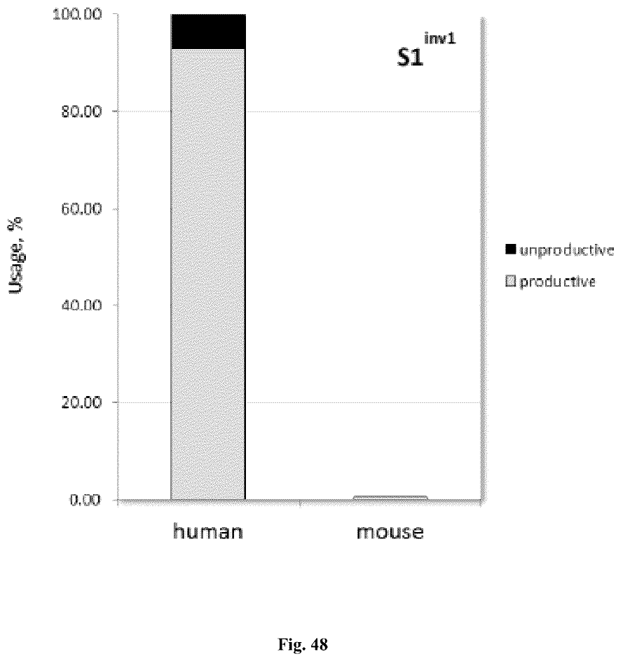

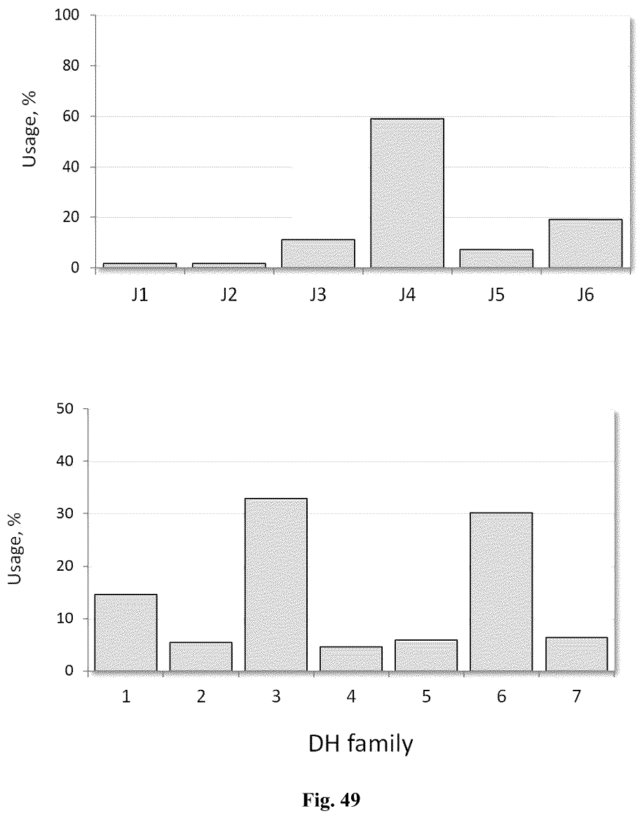

This result indicates that inversion is an effective way to inactivate the rearrangement of endogenous V H gene segments. The S1^{inv1} mouse also shows a similar usage of both D and J_{H} gene segments to human (FIG. 49) (Link, J M et al. Mol. Immunol. 2005. 42, 943-955). Thus, a mouse was produced that comprises a transgenic heavy chain locus that expresses heavy chains comprising human variable regions, but no mouse variable regions, and furthermore the human variable regions demonstrated a normal, human sequence distribution corresponding to human D and J usage observed in humans.

### Example 8

Inactivation of Use of Endogenous IGHV Gene Segments for Expressed Rearranged Heavy Chain By Insertion Of Human IgH Genomic DNA

### INTRODUCTION

Insertion of human BACs with V_{H}-D-J_{H} gene segments into an endogenous mouse heavy chain locus between J_{H}4 and Eμ in chromosome 12 allows human V_{H}-D-J_{H} gene segments to effectively use mouse Eμ and 3′ enhancers and rearrange to generate chimeric antibody with human variable region and mouse constant region. Meanwhile, the endogenous V_{H}-D-J_{H} gene segments are pushed away from endogenous enhancers and constant regions. This distance effect results in inactivation of mouse D and J_{H} use for expressed rearranged antibody products. As the distance increases by stepwise BAC insertion, it is expected that the mouse V_{H} usage would be significantly reduced.

Results

Insertion of human DNA from a 1^{st} human BAC (BAC comprising a the sequence of mouse Chromosome 14 from coordinate 106328951 to coordinate 106494908; containing six most 3′ functional V_{H} gene segments (V_{H}2-5, 7-4-1, 4-4, 1-3, 1-2, 6-1), and all the human D and J_{H} gene segments) into the heavy chain endogenous locus of a AB2.1 ES cell genome between endogenous IGHJ4 and Eμ (at mouse chromosome 12: between coordinates 114666435 and 114666436) effectively inactivates the use of endogenous D and J_{H} gene segments for expressed rearranged immunoglobulin heavy chain (FIG. 44). The rearranged transcripts with mouse V_{H} gene segments are reduced in the resulting S1 mouse. The proportion of transcripts using mouse V_{H} is around 75% of all observed sequences (FIG. 45).

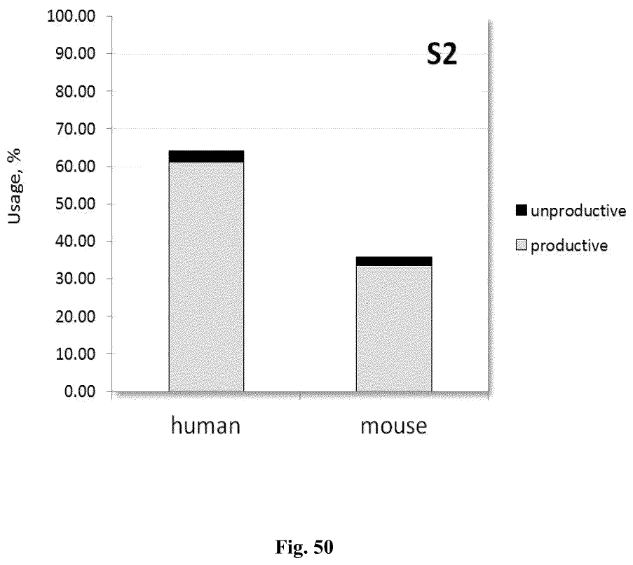

Following the 1^{st} BAC DNA insertion, human DNA from a 2fid human BAC (Chr14: 106494909-106601551) (BAC comprising a the sequence of mouse Chromosome 14 from coordinate 106494909 to coordinate 106601551; containing 5 more functional V_{H} gene segments (V_{H}3-13, 3-11, 3-9, 1-8, 3-7)) was inserted into the landing pad left behind after curing following the 1^{st} BAC insertion (see, eg, FIG. 24). The mouse V_{H} usage is further significantly reduced following this insertion of the 2^{nd} BAC into the locus. The proportion of transcripts using mouse V_{H} was further reduced to 35% of all observed sequences (FIG. 50).

This result indicate that the endogenous V_{H}-D-J_{H} gene segments could be inactivated (ie, not used for expressed rearranged heavy chains) through insertion of human VDJ sequences from one or more BACs. As the distance increases by stepwise BAC insertion, it is expected that the mouse V_{H} usage would be significantly reduced.

### Example 9

Normal Class Switch and Hypermutation in Transgenic Mice of the Invention

### INTRODUCTION

The B cell arm of the immune system has evolved to produce high affinity, antigen-specific antibodies in response to antigenic challenge. Antibodies are generated in B lymphocytes by a process of gene rearrangement in which variable (V), diversity (D; for the IGH locus) and joining (J) gene segments are recombined, transcribed and spliced to a Cμ (for IGH) or a OK or C_{λ} (for IGL) constant region gene segment to form an IgM antibody. Depending on the stage of B cell development, IgM is either located on the cell surface or secreted. The recombination process generates a primary antibody repertoire with sufficient germ line diversity to bind a wide range of antigens. However, it is usually not large enough to provide the high affinity antibodies that are required for an effective immune response to an antigen such as an infectious agent. Therefore, the immune system adopts a two-stage diversification process to increase diversity further. When challenged with antigens, B cells undergo selection and maturation by a process called somatic mutation. B cells expressing antibodies which bind to antigen undergo multiple rounds of diversification, clonal expansion and antigen selection in the germinal centres (GCs) of the secondary lymphoid organs. During this process, the rearranged variable regions of the immunoglobulin genes acquire somatic hypermutation through nucleotide substitution, addition or deletion. This stepwise process creates a secondary repertoire from the weak binders selected originally from the primary repertoire and combines rapid proliferation of antigen-reactive B cells with intense selection for quality of binding, eventually giving rise to high affinity antibodies with broad epitope coverage. During this process, antibodies undergo class switching in which the Cμ constant region is replaced by Cγ, Cα or Cε to produce respectively IgG, A or E classes of antibody with different effector functions.

Insertion of 1^{st} human BAC (Chr14: 106328951-106494908) containing six most 3′ functional V_{H} gene segments (V_{H}2-5, 7-4-1, 4-4, 1-3, 1-2, 6-1), and all the D and J_{H} gene segments into the locus between endogenous IGHJ4 and Eμ (Chr12: 114666435 and 114666436) produces transgenic mice that generate chimeric immunoglobulin heavy chains containing human variable and mouse constant regions. This result demonstrates that human immunoglobulin gene segments are able to be rearranged and expressed in mice. Here, RT-PCR experiments and sequence analysis were performed to further demonstrate that immunized transgenic mice have proper class switch and hypermutation for generated antibodies.

Methods

RT-PCR and sequence analysis: Wild type or S1 chimera mice at 6-8 weeks of age were primed by intraperitoneal injection of 10⁸ sheep RBCs suspended in phosphate buffer saline (PBS). The immunized mice were boosted twice with the same amount of sheep RBCs two and four weeks after priming. Four days after the last boost, peripheral blood cells were collected from the immunized mice. Total RNA was isolated from peripheral blood cells with TRIzol® reagent (lnvitrogen™) and treated with DNase I. Reverse transcription polymerase chain reaction (RT-PCR) was performed using SuperScript® μl First-Strand Synthesis System (Invitrogen™) following the protocol supplied by the manufacturer. The 1^{st} strand cDNA was synthesized with the specific Cγ primers (Cγ1, Cγ2a, Cγ2b), following by PCR with specific human V primers (VH1-2,3, VH4-4, VH6-1) and Cγ primers (Table 2). Following reaction, the RT-PCR product was checked on a 1% agarose gel and purified using QIAquick® Gel Extraction Kit (QIAGEN) as the protocol supplied with the kit, then cloned into pDrive vector using QIAGEN PCR Cloning Kit (QIAGEN) for sequencing analysis.

TABLE 2

| | | |
|---|---|---|
| ELP1352_Cγ1 | 5′-AGAGCGGCCGCTGGGCAA | SEQ ID No 27 |
| | CGTTGCAGGTGACGGTC-3′ | |
| ELP1353_Cγ2b | 5′-AGAGCGGCCGCTTTGTCC | SEQ ID No 28 |
| | ACCGTGGTGCTGCTGG-3′ | |
| ELP1354_Cγ2a | 5′-AGAGCGGCCGCACATTGC | SEQ ID No 29 |
| | AGGTGATGGACTGGC-3′ | |
| ELP1356_VH4-4 | 5′-AGGACGCGTGAAACACCT | SEQ ID No 30 |
| | GTGGTTCTTCCTCCTGC-3′ | |
| ELP1357_VH1-2, 3 | 5′-AGGACGCGTCACCATGGA | SEQ ID No 31 |
| | CTGGACCTGGAGGAT-3′ | |
| ELP1358_VH6-1 | 5′-AGGACGCGTATGTCTGTC | SEQ ID No 32 |
| | TCCTTCCTCATCTTCC-3′ | |

Results

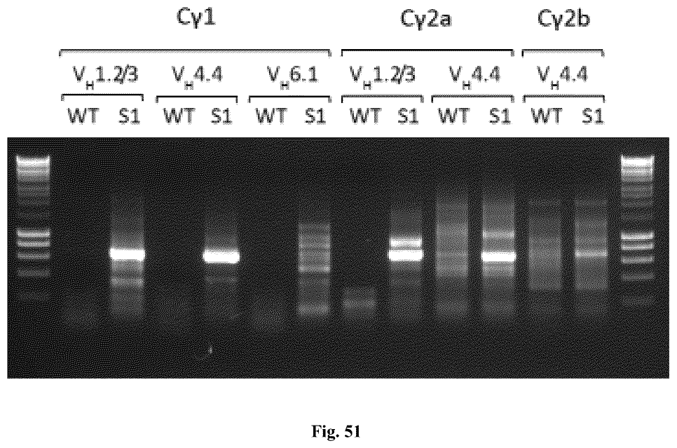

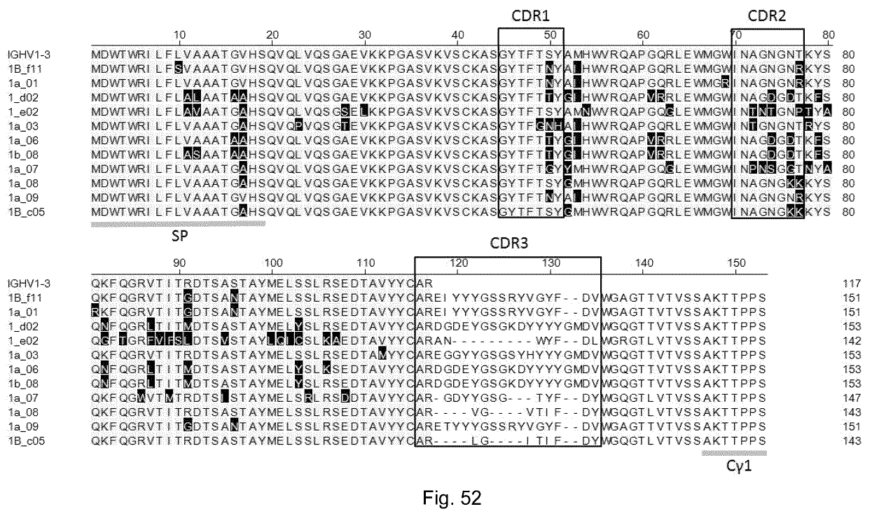

The rearranged transcripts were detected using RT-PCR with human V_{H}-specific and mouse Cγ-specific primers for amplification from peripheral blood cells of immunized transgenic mice (FIG. 51). Further sequence analysis of these amplified fragments demonstrated hypermutation happened within the human variable regions of these IGγ chains (FIG. 52). These results indicate that loci of the invention comprising insertion of human IGH BAC containing V_{H}, D and J_{H} gene segments into the locus between endogenous IGHJ4 and Eμ regions has normal class switching and hypermutation functionality (IgM to IgG) following antigen challenge.

### Example 10

Normal B Cell Compartments in Transgenic Mice of the Invention

### INTRODUCTION

In mice, about 2×10 7 bone marrow immature B cells are produced daily. Among them, only 10-20% of these cells survive to exit the bone marrow and enter the spleen. The immature splenic B cell population is divided into two distinct subsets: transitional 1 (T1) and transitional 2 (T2) B cells. In vivo experiments indicate that T1 cells give rise to T2 cells, whereas T2 cells can further differentiate into mature (M) B cells. In contrast to immature B cells (3-4 days old), mature B cells are long-lived (15-20 weeks old) and are ready to respond to antigens (Pillai S et al; Immunol. Reviews. 2004. 197: 206-218). Thus, the component of mature B cell population is directly linked to the efficiency of humoral immune response.

The T1, T2 and M cell populations can be categorized by their cell surface IgM and IgD levels. A normal phenotype of splenic B cell compartment is required to mount a robust immune response.

Methods

Flow cytometric analysis of mature B lymphocytes: To obtain a single cell suspension from spleen, the spleens of mice listed below were gently passaged through a 30 μm cell strainer. Single cells were resuspended in PBS supplemented with 3% heat inactivated foetal calf serum (FCS; Gibcog. The following antibodies were used for staining:

Antibody against B220/CD45R conjugated with allophycocyanin (APC) (eBioscience, clone RA3-6B2), antibody against IgD receptor conjugated with phycoerythrin (PE) (eBioscience, clone 11-26) and IgM receptor conjugated with fluorescein isothiocyanate (FITC) (eBioscience, clone 11/41).

5×10⁶ cells were used for each staining. To each vial containing splenocytes a cocktail of antibodies was added consisting of: IgD (PE) (eBioscience, clone 11-26), IgM (FITC) and B220/CD45R (APC). Cells were incubated at 6′C for 15 minutes, washed to remove excess of unbound antibodies and analysed using a fluorescence-activated cell sorting (FACS) analyser from Miltenyi Biotech. B-cells were gated as B220⁺ IgM⁺ IgD⁻ for T1 population, B220⁺ IgM⁺ IgD⁺ for T2 population and B220 1⁺ IgM⁻ IgD⁺ for M population. Percentage of cells was calculated using gating system.

Results

Four Different Genotypes of Mice were Generated:
A transgenic mouse homozygous for a heavy chain transgene comprising insertion of the 1^{st} BAC human DNA noted above in which there are 6 human VH, all functional human D and JH gene segments(S1/S1);
A transgenic mouse homozygous for a heavy chain transgene comprising insertion of a human VH, all functional human D and JH gene segments (H1/H1); and
A transgenic mouse homozygous for a kappa chain transgene comprising insertion of 6 functional human Vκ and 5 functional JK gene segments (K1/K1).

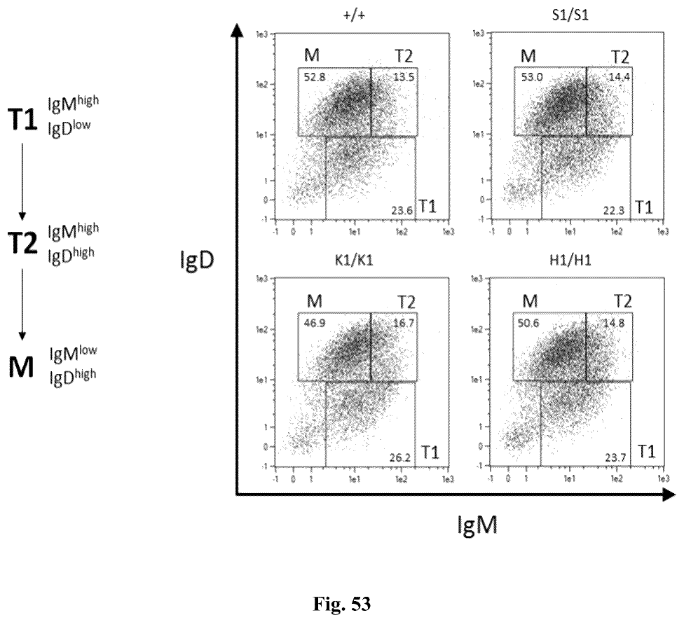

Spleens from these naïve mice were collected and analysed for their B cell compartments. The number and percentages of T1, T2 and M cells among those mice are similar (FIG. 53), indicating that genetic manipulation of endogenous IG loci in transgenic mice according to the invention do not compromise their B cell development. These data help to establish that animals according to the invention provide a robust platform for antibody discovery.

### Example 11

Normal IgH Isotypes & Serum Levels in Transgenic Animals of the Invention

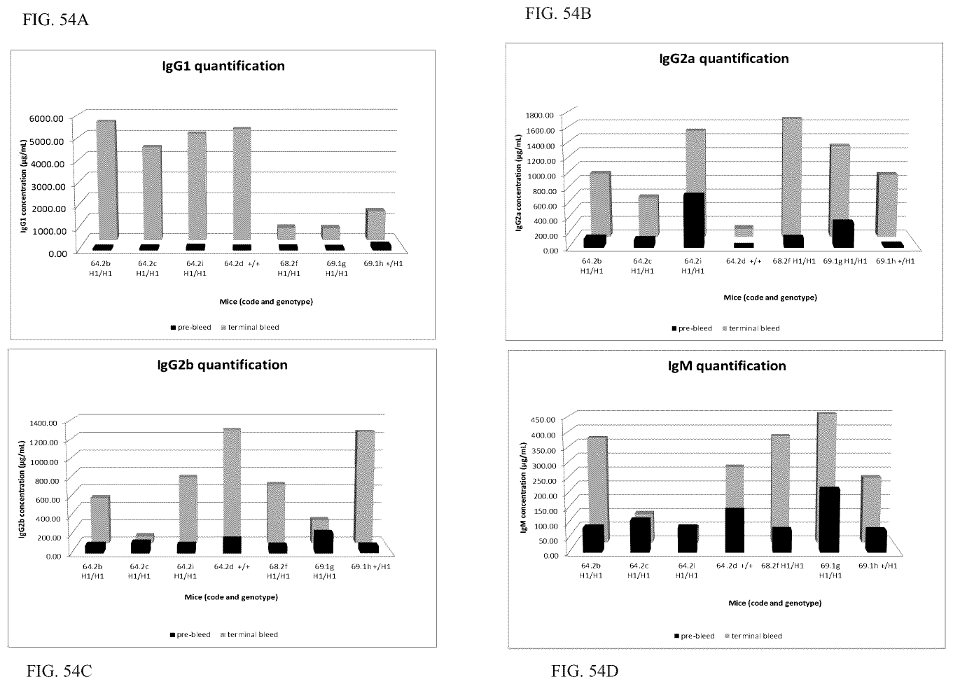

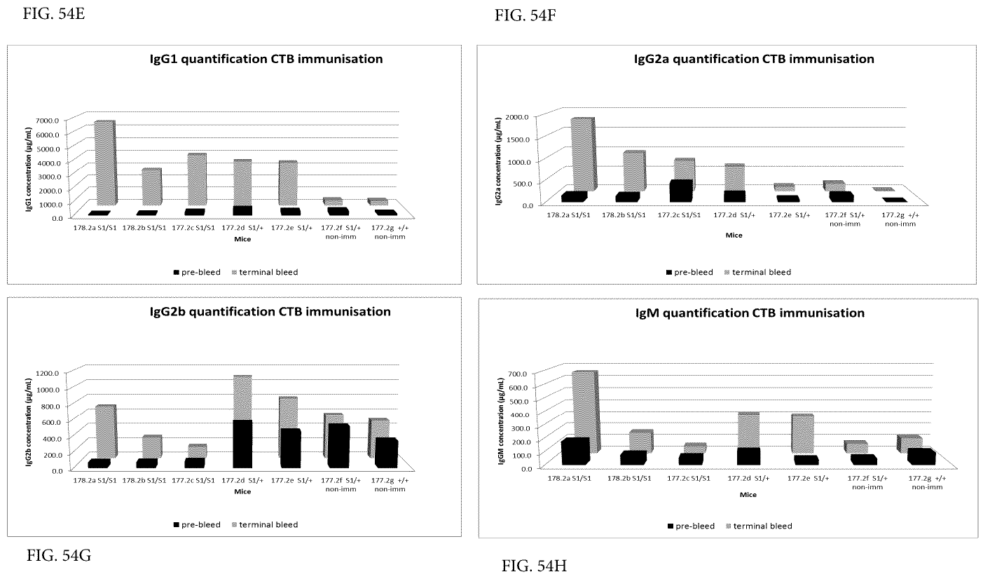

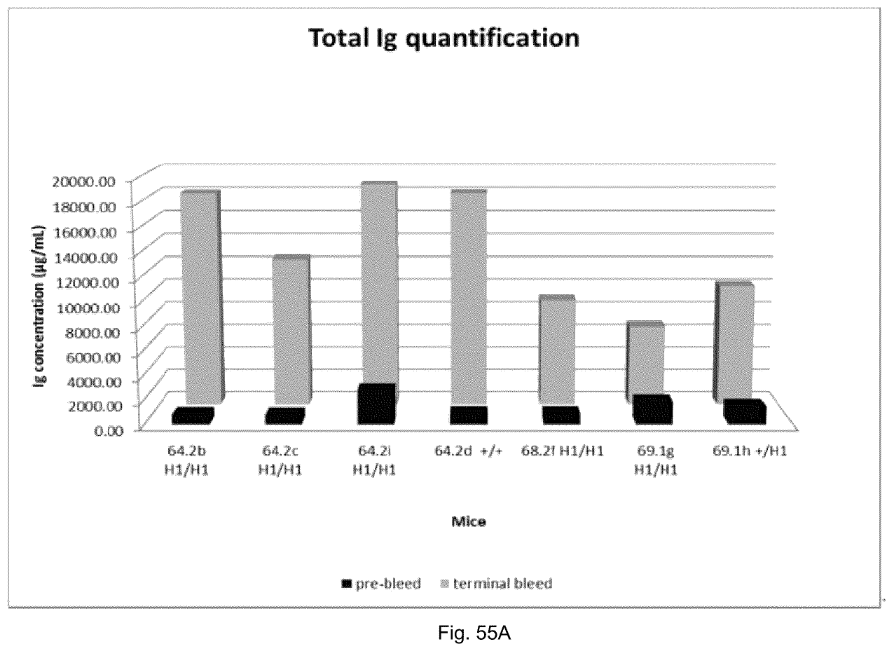

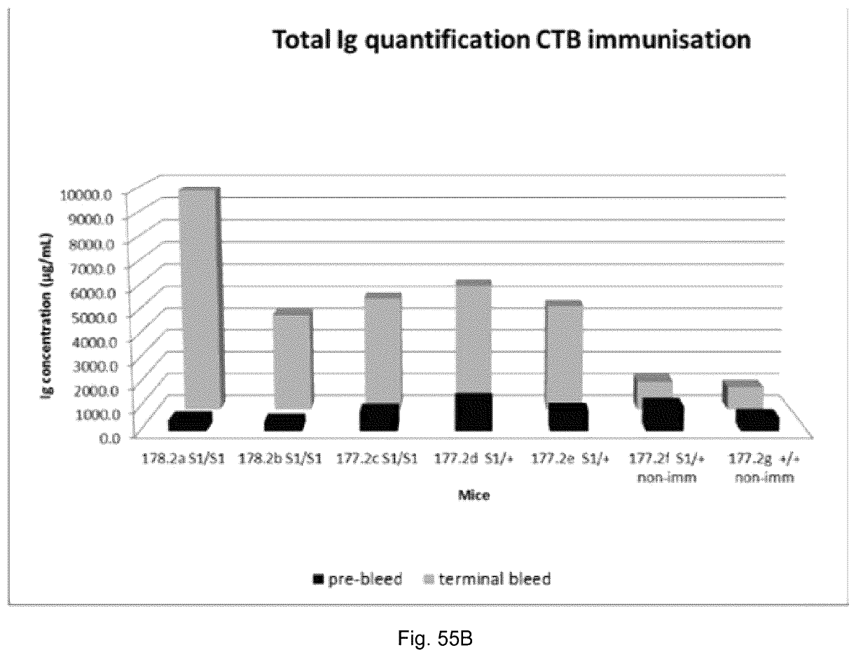

Transgenic mice (H1) carrying all human J_{H}, all human DH and human Vh2-5 under control of a rat switch region or mice (S1) carrying all human J_{H}, all human DH and human Vh2-5, Vh7-41, Vh4-4, Vh1-3, Vh1-2 and Vh6-1 under control of a mouse switch region were immunised with 100 μg Cholera Toxin B subunit (CTB; Sigma-Aldrich® 09903) emulsified in Complete Freund's Adjuvant CFA; Sigma-Aldrich® F 5881). At least three animals were injected sc or ip and then boosted with 25 μg antigen in Incomplete Freund's Adjuvant (IFA; Sigma-Aldrich® F 5506) at (i) 14 days and 21 days or (ii) 28 days after priming. Blood was taken before priming at day “−1” (pre-bleeds) and on the day the spleens were taken (usually 4d after last boost). Serum was analysed by ELISA using an antigen independent assessment of Ig isotypes. This assay detects total serum antibodies of all species. Specific detection for mouse IgG1,1gG2a, IgG2b and IgM was used ((Anti-mouse IgG1 HRP AbD Serotec STAR132P, Anti-mouse IgG2a HRP AbD Serotec STAR133P, Anti-mouse IgG2b HRP AbD Serotec STAR134P, Anti-mouse IgM HRP Abcam® ab97230) and concentrations were read off a standard curve produced for each isotype using polyclonal isotype controls (IgG1, Kappa murine myeloma Sigma-Aldrich® M9269, IgG2a, Kappa murine myeloma Sigma-Aldrich® M9144, IgG2b, Kappa from murine myeloma Sigma-Aldrich® M8894, IgM, Kappa from murine myeloma Sigma-Aldrich® M3795). Results (FIGS. 54 & 55 for H1 homozygous and S1 homozygous and heterozygous mice) showed that even with these relatively short immunisation regimes mice showed an increase in overall IgG levels after immunisation over pre-bleeds. In cases where control mice (+/+) not carrying any human immunoglobulin genes were included and immunised, these mice showed comparable changes in total observed Ig levels (FIG. 54). Individual isotype levels were more variable between animals possibly showing various stages of class switching. IgM levels never exceeded 800 μg/ml whereas IgG levels reached more than 6 mg/ml in some animals. Non-immunised controls showed no such increases in switched isotype Ig levels.

These results demonstrate that mice comprising multiple human VDJ gene segments under the control of a rat Sp rat or mouse switch are able to undergo productive recombination and class switching in response to antigen challenge and that the mice produce antibody levels that are broadly comparable to unmodified mice The transgenic mice are able to produce antibodies of each of the IgG1, IgG2a, IgG2b and IgM isotypes after immunisation. Titers for CTB-specific Ig in pre-bleeds and terminal bleeds were determined and all immunised animals showed at CTB-specific titres of at least 1/100 000.

### Example 12

Generation of Anti-Ovalbumin Antibodies with Sub-50 nm Affinities From Animals of the Invention

Transgenic mice carrying all human JH, all human DH and human Vh2-5 under control of a rat Sp switch region were immunised with 25 μg ovalbumin (OVA; Sigma-Aldrich® A7641) in Sigma-Aldrich® adjuvant (Sigma Adjuvant System® 56322) ip and then boosted with the same amount of OVA in adjuvant at day 14 and day 21. Spleenocytes were taken 4 days later and fused using 1 ml polyethyleneglycol (PEG Average MW1450; Sigma-Aldrich® P7306) with a myeloma line. Fused hybridoma cells were plated on 5 96-well plates and after selection with hypoxanthine-aminopterin-thymidine (HAT) wells tested for expression of OVA-specific antibodies by ELISA. Clones positive by ELISA were re-tested by surface plasmon resonance (SPR) and binding kinetics determined using the ProteOn™ XPR36 (Bio-Rad®). Briefly, anti-mouse IgG (GE Biacore™ BR-1008-38) was coupled to a GLM biosensor chip by primary amine coupling, this was used to capture the antibodies to be tested directly from tissue culture supernatants. Ovalbumin was used as the analyte and passed over the captured antibody surface at 1024 nM, 256 nM, 64 nM, 16 nM, 4 nM with a 0 nM (i.e. buffer alone) used to double reference the binding data. Regeneration of the anti-mouse IgG capture surface was by 10 mM glycine pH1.7, this removed the captured antibody and allowed the surface to be used for another interaction. The binding data was fitted to 1:1 model inherent to the ProteOn™ XPR36 analysis software. The run was carried out 1×HBS-EP (10 mM Hepes, 150 mM NaCl, 3 mM EDTA, 0.05% polysorbate, pH7.6 (Teknova H8022)) used as running buffer and carried out at 25° C.

For 8 positive clones, heavy chain V-regions were recovered by RT-PCR (Access RT-PCR System, A1250, Promega) using forward primers specific for Ig signal sequences (Wardemann et a/Science 301, 1374 (2003)) and the following reverse primers for the constant regions of mouse IgG (Table 3):

TABLE 3

| Primer Name | Sequence | bp | |
|---|---|---|---|
| migG1_2 rev | GGGGCCAGTGGATAGACAGAT | 21 | SEQ ID No 33 |
| migG2b rev | CAGTGGATAGACTGATGG | 18 | SEQ ID No 34 |
| migG2a_2 rev | CAGTGGATAGACCGATGG | 21 | SEQ ID No 35 |
| mCH1 unirev - | KCAGGGGCCAGTGGATAGAC | 20 | SEQ ID No 36 |
| mCH1 unirev_2 | TARCCYTTGACMAGGCATCC | 20 | SEQ ID No 37 |

RT-PCR products were either directly sequenced using the same primer pairs or cloned in to TA plasmids (TOPO® TA Cloning® Kit for Sequencing, K4595-40, lnvitrogen™) and submitted for plasmid sequencing. Results (Table 4, below) show that CDRH3 sequences had variable CDRs except for two identical clones (16C9 and 20B5) that also had near identical KD kinetic values. The determined equilibrium binding constant KD ranged from 0.38 nM to 40.60 nM, as determined by SPR at 25° C.

These results demonstrate that mice comprising multiple human VDJ gene segments under the control of a rat Cμ switch are able to undergo productive recombination and produce high affinity antigen-specific antibodies whose CDR3 regions have sequences encoded by human gene segments (human JH was separately identified by V-Quest, IMGT).

TABLE 4

| | | CDR3 and FR4 (underlined) | |
|---|---|---|---|
| KD | clone | according to Kabat | |
| [nM] | code | definition | |
| 0.38 | 16C9 | QEVINYYYYGMDVWGQGTTVTVSS | SEQ ID No 38 |
| 0.52 | 20B5 | QEVINYYYYGMDVWGQGTTVTVSS | SEQ ID No 39 |
| 5.89 | 19F4 | LEMATINYYYYGMDVWGQGTMVTVSS | SEQ ID No 40 |
| 39.70 | 19E1 | QEFGNYYYYGMDVWGQGTTVTVSS | SEQ ID No 41 |
| 3.10 | 19G8 | QEDGNPYYFGMDFWGQGTTVTVSS | SEQ ID No 42 |
| 8.95 | 20H10 | GSSYYYDGMDVWGQGTTVTVSS | SEQ ID No 43 |
| 4.46 | 18D10 | LENDYGYYYYGMDVWGQGTTVTVSS | SEQ ID No 44 |
| 40.60 | 16F2 | RGGLSPLYGMDVWGQGTTVTVSS | SEQ ID No 45 |

### Example 13

Generation of Anti-Cholera Toxin B Antibodies With Human Vh Regions from Animals of the Invention

Transgenic mice carrying all human JH, all human DH and human Vh2-5, Vh7-41, Vh4-4, Vh1-3, Vh1-2 and Vh6-1 under control of a mouse Sp switch region were immunised and fused as described in Example 11. Fused hybridoma cells were plated on 5 96-well plates and after selection with hypoxanthine-aminopterin-thymidine (HAT) or G418 (Gibco® Cat No 10131-027, Lot 503317) and wells tested for expression of CTB-specific antibodies by ELISA. Clones positive by ELISA were re-tested by surface plasmon resonance SPR and binding kinetics determined using the ProteOn XPR36™ (Bio-Rad®).

Briefly, anti-mouse IgG (GE Biacore™ BR-1008-38) was coupled to a GLM biosensor chip by primary amine coupling, this was used to capture the antibodies to be tested directly from tissue culture supernatants. Cholera toxin B was used as analyte and passed over the captured antibody surface at 256 nM, 64 nM, 16 nM, 4 nM and 1 nM, with a 0 nM (i.e. buffer alone) used to double reference the binding data. Regeneration of the anti-mouse IgG capture surface was by 10 mM glycine pH1.7, this removed the captured antibody and allowed the surface to be used for another interaction. The binding data was fitted to 1:1 model inherent to the ProteOn XPR36™ analysis software. The run was carried out 1×HBS-EP (10 mM Hepes; 150 mM NaCl, 3 mM EDTA, 0.05% polysorbate, pH7.6 (Teknova H8022)) used as running buffer and carried out at 37° C.

From the clones initially identified by ELISA, binding to CTB was confirmed by SPR. However, due to the pentameric nature of the cholera toxin B, the majority of fits to the 1:1 model were poor and the equilibrium binding constant KDs could not be accurately determined. Where fits were acceptable, equilibrium binding constant KDs determined ranged from 0.21 nM to 309 nM but due to the pentameric nature of cholera toxin B these are likely to be the result of multimeric interactions and therefore apparent affinities with possible avidity components.

Clones identified by SPR for binding to CTB were subjected to RT-PCR as described in Example 12 to recover the Vh regions. RT-PCR products were directly sequenced using the same primer pairs. Results were obtained for only 14 clones presumably because the human primers described in Wardemann et a/were not designed to amplify mouse Vh regions and therefore may have failed to amplify certain mouse Vh classes. Results showed that 3 of the 14 CTB-specific recovered heavy chain V-region sequences were human V, D and J regions as identified by V-Quest, IMGT (Table 5).

TABLE 5
Alignment of heavy chain CDRs and J-region of 3 clones identified
as binding to CTB and preferentially matching with human
reference sequences from IMGT database; note that the KD values given
here are apparent values due to the avidity of the CTB- antibody interaction

| Vh | Clone | Sequence Kabat definitions | | | | | KD |
|---|---|---|---|---|---|---|---|
| region | Name | CDR1 | CDR2 | CDR3 | | J-regions | [MM] |
| IGHV4- | — | SSNWWS | EIYHSGSTNYNPSL | n/a | IGHJ2*01 | YWYFDLWGRGTLVT | — |
| 4*02 | | (SEQ ID NO 51) | KS | | | VSS | |
| | | | (SEQ ID NO 56) | | | (SEQ ID NO 64) | |
| | 12D10 | SGNWWS | EIYHSGTNYNPSLK | GPLTGEKYYFDL | | -YYFDLWGRGTLVT | 0.27 |
| | | (SEQ ID NO 52) | S | (SEQ ID NO 61) | | VSS | |
| | | | (SEQ ID NO 57) | | | (SEQ ID NO 65) | |
| | 1283 | RSNWWS | EIYHSGSTNYNPSL | IGDWYFDL | | -WYFDLWGRGTLVT | 0.85 |
| | | (SEQ ID NO 53) | KS | (SEQ ID NO 62) | | VSS | |
| | | | (SEQ ID NO 58) | | | (SEQ ID NO 66) | |
| IGHV6- | — | SNSAAWN | RIYYRSKWYNDYAV | n/a | IGHJ3*01 | DAFDVWGQGTMVTV | — |
| 1*01 | | (SEQ ID NO 54) | SVKS | | | SS | |
| | | | (SEQ ID NO 59) | | | (SEQ ID NO 67) | |
| | 4A12 | SNSAAWN | RTYYRSKWYNDYKV | EGSHSGSGWYLDAF | | DAFDIWGQGTKVTV | |
| | | (SEQ ID NO 55) | SVKS | DI | | SS | |
| | | | (SEQ ID NO 60) | (SEQ ID NO 63) | | (SEQ ID NO 68) | |

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

1. A method of providing an antigen-specific polypeptide comprising a human IgH variable region, an antigen-specific antibody comprising said polypeptide, a cell producing said polypeptide, a nucleic acid encoding said polypeptide or said variable region and/or a biological sample comprising said polypeptide, the method comprising:
isolating from a transgenic mouse that produces antibody specific for said antigen one or more of said polypeptide, said antigen-specific antibody comprising said polypeptide, said cell producing said polypeptide, said nucleic acid encoding said polypeptide or said variable region and/or said biological sample comprising said polypeptide, wherein said transgenic mouse has a germline comprising a homozygous immunoglobulin heavy chain (IgH) locus comprising a plurality of unrearranged human IgH V gene segments, D gene segments and JH gene segments at an endogenous IgH locus and a constant (C) region comprising an endogenous IgH C gene segment;
wherein said homozygous IgH locus comprises in 5′ to 3′ transcriptional orientation:
(i) unrearranged human immunoglobulin heavy chain (IgH) variable region (VH) DNA comprising human IgH V gene segments, human D gene segments and human JH gene segments comprising a human 3′ JH gene segment,
(ii) a chimeric JC intron comprising (a) human JC intronic DNA downstream of said human 3′ J_{H} gene segment and (b) truncated mouse JC intronic DNA, and
(iii) a mouse Cμ enhancer and said C region,
wherein said plurality of unrearranged human V gene segments, D gene segments and J gene segments in said IgH locus are operatively linked to said C region such that said IgH locus of said mouse is capable of undergoing V, D, J joining of said unrearranged human IgH variable region gene segments,
such that said transgenic mouse is capable, upon stimulation with an antigen, of producing a plurality of antibodies specific for said antigen comprising a chimeric Ig heavy chain comprising a human VH region and a mouse C region;
wherein said genome comprises all or part of mouse IgH variable region DNA upstream of human VH gene segments, wherein expression of mouse Ig heavy chain comprising a mouse IgH variable region and a mouse IgH constant region is inactive in said mouse.

2. The method of claim 1, wherein said human 3′ JH is less than 2 kb upstream of said mouse JC intronic DNA.

3. The method of claim 1, wherein said mouse comprises IgH-VDJCμ transcripts encoding IgH polypeptides comprising CDR-H3 lengths of 17, 18 and 19 amino acids, wherein the mean frequency of the group consisting of said transcripts encoding CDR-H3 lengths of 17, 18 and 19 amino acids is between 5% and 10%.

4. The method of claim 3, wherein said mouse further comprises IgH-VDJCμ transcripts encoding IgH polypeptide comprising CDR-H3 lengths selected from the group consisting of: 20, 21 and 22 amino acids.

5. The method of claim 1, wherein said mouse JC intronic DNA comprises DNA of an IgH locus of a 129 strain mouse.

6. The method of claim 1, further comprising obtaining B cells expressing an antigen-specific antibody comprising a chimeric immunoglobulin heavy chain polypeptide comprising a human heavy chain variable (VH) region and a mouse constant (C) region, comprising isolating B cells expressing said antigen-specific antibody from said transgenic mouse that has been contacted with an antigen under conditions sufficient to generate antibody specific to said antigen.

7. The method of claim 6, further comprising the step of culturing said B cells, and isolating antigen-specific antibody from said cultured B cells.

8. The method of claim 6, further comprising the step of isolating from said B cells nucleic acid encoding said human VH region and said mouse C region of said chimeric immunoglobulin heavy chain polypeptide.

9. The method of claim 8, further comprising the step of replacing in said nucleic acid encoding said human VH region and said mouse C region of said chimeric immunoglobulin heavy chain polypeptide, the portion encoding said mouse C region with nucleic acid encoding a human C region, thereby forming a nucleic acid encoding said human VH region and said human C region.

10. The method of claim 6, further comprising isolating an antibody that binds said antigen, comprising
(i) immortalizing said isolated B cells expressing said antigen-specific antibody or progeny thereof, optionally producing hybridomas therefrom; and
(ii) isolating an antibody expressed by the immortalized B cells of (i).

11. The method of claim 1, wherein said mouse is naïve.

12. The method of claim 1, wherein said plurality of unrearranged human D gene segments and said plurality of unrearranged human JH gene segments comprises all human D (D) gene segments and all human heavy chain J (JH) gene segments.

13. The method of claim 12, wherein said plurality of unrearranged human VH gene segments comprises all human VH gene segments.

14. The method according to claim 1, further comprising one or more steps of the group consisting of:
(a) isolating nucleic acid encoding a said antibody, said IgH polypeptide, and/or a human VH region thereof,
(b) isolating said antibody, said IgH polypeptide, and/or a human VH region thereof,
(c) isolating a cell comprising said nucleic acid of step (a)
(d) isolating a cell comprising said antibody, said IgH polypeptide, and/or a human VH region thereof, and
(e) isolating a biological sample comprising nucleic acid encoding and/or a cell comprising said human VH region.

15. The method of claim 1, wherein said antigen comprises a vaccine.

16. The method of claim 1, wherein said transgenic mouse is capable of breeding with a second transgenic mouse, said second transgenic mouse having a genome comprising a homozygous IgH locus comprising unrearranged human IgH variable region gene segments at an endogenous IgH locus upstream of and operably linked to a constant (C) region comprising an endogenous C segment of an IgH locus, to provide subsequent generation mice comprising in their genome a homozygous IgH locus comprising unrearranged human IgH variable region gene segments at an endogenous IgH locus upstream of a constant (C) region comprising an endogenous C segment of an IgH locus and operably linked to the C region.
